(19) United States
(12) Patent Application Publication
(10) Pub. No.: US 2021/0284742 A1
(43) Pub. Date: Sep. 16, 2021

(54) METHODS OF TREATING OR PREVENTING MUCORMYCOSIS

(71) Applicants: University of Maryland, Baltimore, Baltimore, MD (US); Los Angeles Biomedical Research Institute at Harbor at UCLA Medical Center, Torrance, CA (US)

(72) Inventors: Vincent Bruno, Baltimore, MD (US); Tonya N. Watkins, Baltimore, MD (US); Ashraf Ibrahim, Irvine, CA (US)

(21) Appl. No.: 17/255,570

(22) PCT Filed: Jun. 28, 2019
(86) PCT No.: PCT/US19/39870

§ 371 Date: Dec. 23, 2020

Related U.S. Application Data
(60) Provisional application No. 62/692,058, filed on Jun. 29, 2018.
(60) Provisional application No. 62/772,893, filed on Nov. 29, 2018.
(60) Provisional application No. 62/843,805, filed on May 6, 2019.

(51) Int. Cl. C07K 16/28; A61K 31/5377; A61K 31/519; A61K 31/517; A61K 31/506; A61K 39/395; A61K 31/569; A61K 31/7048; A61K 31/4439; A61K 31/496; A61P 31/10

(52) U.S. Cl. CPC C07K 16/2863; A61K 31/5377; A61K 31/519; A61K 31/517; A61K 31/506; A61P 31/10; A61K 31/569; A61K 31/7048; A61K 31/4439; A61K 31/496; A61K 39/3955

(57) ABSTRACT

The invention provides a method of treating or preventing mucormycosis in a subject in need thereof, comprising administering to the subject a therapeutically-effective amount of an agent that inhibits a signaling pathway of a receptor selected from the group consisting of epidermal growth factor receptor, platelet derived growth factor receptor, ErbB2/Her2, progesterone receptor and a combination thereof.

### CROSS-REFERENCE TO RELATED APPLICATIONS

This application claims the benefit of U.S. Provisional Appl. No. 62/692,058, filed on Jun. 29, 2018, U.S. Provisional Appl. No. 62/772,893, filed on Nov. 29, 2018, and U.S. Provisional Appl. No. 62/843,805, filed on May 6, 2019, the contents of which are hereby incorporated by reference in their entirety.

### STATEMENT OF FEDERALLY SPONSORED RESEARCH AND DEVELOPMENT

This invention was made with government support under Grant Numbers AI110820 and AI063503 awarded by the National Institutes of Health. The government has certain rights in the invention.

### FIELD OF THE INVENTION

The field of the invention relates generally to the field of medicine, pharmaceuticals and infectious disease, and in particular to compositions and methods for treating or preventing mucormycosis.

### BACKGROUND OF THE INVENTION

Mucormycosis, a NIAID-classified emerging disease, is a deadly invasive fungal infection. Mucormycoses are invasive fungal infections of humans caused by species of the order Mucorales, subphylum Mucormycotina. Rhizopus spp. are the most common organisms isolated from patients with mucormycosis and are responsible for ˜70% of all cases (Ribes, J. A., Vanover-Sams, C. L. & Baker, D. J. Zygomycetes in human disease. Clin Microbiol Rev 13, 236-301 (2000)). The major risk factors include uncontrolled diabetes mellitus that results in hyperglycemia and ketoacidosis (DKA), other forms of acidosis, treatment with corticosteroids, solid organ or bone marrow transplantation, neutropenia, trauma and burns (e.g., wounded soldiers in Iraq and Afghanistan), malignant haematological disorders, and deferoxamine therapy in patients receiving haemodialysis (Ibrahim, A. S. & Kontoyiannis, D. P. Update on mucormycosis pathogenesis. Curr Opin Infect Dis 26, 508-515 (2013); Weintrob, A. C. et al. Combat trauma-associated invasive fungal wound infections: epidemiology and clinical classification. Epidemiol Infect 143, 214-224, doi:10.1017/5095026881400051X (2015); Sugar, A. M. in Principles and Practice of Infectious Diseases Vol. 2 (eds G. L. Mandell, J. E. Bennett, & R. Dolin) 2973-2984 (Elsevier Churchill Livingstone, 2005)).

The most common forms of mucormycosis, based on anatomical site, are rhino-orbital/cerebral, pulmonary, cutaneous, gastrointestinal and disseminated. Rhino-orbital/cerebral mucormycosis is found almost exclusively in DKA patients while pulmonary disease is mainly found in neutropenic patients (Kwon-Chung K J, Bennett J E. 1992. Mucormycosis, p 524-559, Medical Mycology. Lea & Febiger, Philadelphia). Cutaneous necrotizing mucormycosis outbreaks in healthy individuals have also been reported and often follow natural disasters or severe trauma (e.g. infections following the tsunami that devastated South East Asia in 2004 and the tornadoes that occurred in Joplin, Mo., USA in June 2011) (Neblett Fanfair R, Benedict K, Bos J, Bennett S D, Lo Y C, Adebanjo T, Etienne K, Deak E, Derado G, Shieh W J, Drew C, Zaki S, Sugerman D, Gade L, Thompson E H, Sutton D A, Engelthaler D M, Schupp J M, Brandt M E, Harris J R, Lockhart S R, Turabelidze G, Park B J. Necrotizing cutaneous mucormycosis after a tornado in Joplin, Mo., in 2011. N Engl J Med 367:2214-25; Andresen D, Donaldson A, Choo L, Knox A, Klaassen M, Ursic C, Vonthethoff L, Krilis S, Konecny P. 2005. Multifocal cutaneous mucormycosis complicating polymicrobial wound infections in a tsunami survivor from Sri Lanka. Lancet 365:876-8). Since there are no federal requirements to report fungal infections, the true prevalence of mucormycosis is likely to be much higher than currently reported.

There are only two antifungal agents approved by the USA FDA for treating mucormycosis as first-line therapy; the first is amphotericin B (AmB) which has been used for treating mucormycosis for the last 6 decades. AmB has serious nephrotoxicity, other adverse effects and very limited clinical success (Spellberg, B., Edwards Jr., J. & Ibrahim, A. Novel perspectives on mucormycosis: pathophysiology, presentation, and management. Clin Microbiol Rev 18, 556-569 (2005); Marty, F. M. et al. Isavuconazole treatment for mucormycosis: a single-arm open-label trial and case-control analysis. Lancet Infect Dis 16, 828-837, doi:10.1016/S1473-3099(16)00071-2 (2016)). Isavuconazole was recently approved to treat mucormycosis and is not superior to AmB-treatment (Marty, F. M. et al. Isavuconazole treatment for mucormycosis: a single-arm open-label trial and case-control analysis. Lancet Infect Dis 16, 828-837, doi:10.1016/S1473-3099(16)00071-2 (2016)). In the absence of surgical removal of the infected focus (e.g. excision of the eye in patients with rhinocerebral mucormycosis), antifungal therapy alone is rarely curative. Posaconazole is appropriate as salvage therapy (Greenberg, R. N. et al. Posaconazole as salvage therapy for zygomycosis. Antimicrob Agents Chemother 50, 126-133, doi:50/1/126 [pii]). Even when highly disfiguring surgical debridement is combined with high-dose antifungal therapy, the mortality associated with mucormycosis is >50%. In patients with prolonged neutropenia, disseminated disease, or cerebral involvement, mortality is 90-100% (Gleissner, B., Schilling, A., Anagnostopolous, I., Siehl, I. & Thiel, E. Improved outcome of zygomycosis in patients with hematological diseases? Leuk Lymphoma 45, 1351-1360 (2004); Kauffman, C. A. Zygomycosis: reemergence of an old pathogen. Clin Infect Dis 39, 588-590, doi:10.1086/422729 (2004)).

In the case of pulmonary mucormycosis, infection is generally acquired by inhalation of spores that are ubiquitous in nature. As lung epithelial cells are among the first host cells that interact with Mucorales spores during pulmonary infection, a molecular understanding of how these cells sense and respond to the pathogen is essential to understanding the pathogenesis of pulmonary mucormycosis. While the invasion of endothelial cells by Rhizopus arrhizus is known to be mediated by the interaction between the fungal-encoded CotH3 and the host-encoded GRP78 (Gebremariam T, Liu M, Luo G, Bruno V, Phan Q T, Waring A J, Edwards J E, Jr., Filler S G, Yeaman M R, Ibrahim A S. 2014. CotH3 mediates fungal invasion of host cells during mucormycosis. J Clin invest 124:237-50; Liu M, Spellberg B, Phan Q T, Fu Y, Fu Y, Lee A S, Edwards J E, Jr., Filler S G, Ibrahim A S. 2010. The endothelial cell receptor GRP78 is required for mucormycosis pathogenesis in diabetic mice. J Clin Invest 120:1914-24), the specific interactions that govern adherence and invasion of lung epithelial cells are poorly understood.

Accordingly, in view of the high mortality rate, extreme morbidity, and limited treatment options, it is imperative to develop alternative strategies to treat and prevent mucormycosis.

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

Described in further detail herein is an unbiased survey of the host transcriptional response during early stages of R. arrhizus var. delemar (R. delemar) infection in a murine model of pulmonary mucormycosis using RNA-seq. Network analysis revealed the activation of the host epidermal growth factor receptor (EGFR) signaling. Consistent with the RNA-seq results, the phosphorylated, activated form of EGFR co-localized with R. delemar spores during in vitro infection of human alveolar epithelial cells. Inhibition of EGFR signaling with Cetuximab or Gefitinib, specific FDA-approved inhibitors of EGFR, significantly reduced the ability of R. delemar to invade and damage airway epithelial cells. Furthermore, Gefitinib treatment significantly prolonged survival of mice with pulmonary mucormycosis. The results provided herein show (1) that EGFR, platelet derived growth factor receptor (PDGFR), ErbB2/HER2, and progesterone receptor (PGR) represent novel host targets to block invasion of cells by R. delemar and (2) that inhibition of these signaling pathways provide novel approaches for treating or preventing mucormycosis. By way of non-limiting examples, FDA-approved drugs such as cetuximab and gefitinib can be repurposed to treat or prevent mucormycosis.

In some embodiments, provided herein are methods of treating or preventing mucormycosis by administering to a subject in need thereof a compound that binds to a growth factor receptor on the surface of the subject's cells or to a progesterone receptor. Such growth receptors include the epidermal growth factor receptor (EGFR), platelet-derived growth factor receptor (PDGFR), and ErbB2/HER2. Compounds that bind the foregoing receptors, including examples of compounds exemplified herein, can be used to treat or prevent mucormycosis. Such compounds include, but are not limited to, antibodies, small-molecule agonists/antagonists, antagonistic peptides, and the like.

In one aspect, the invention described herein provides safe and efficacious treatments, by providing methods for treating and/or preventing mucormycosis by inhibiting EGFR, PDGFR, ErbB2/HER2, and progesterone receptor function. For example, FDA-approved drugs such as cetuximab or gefitinib, either alone or in combination together or with other drugs can be used to treat or prevent mucormycosis in patients in need thereof.

In another aspect, the invention provides a method of treating or preventing mucormycosis in a subject in need thereof, comprising administering to the subject a therapeutically-effective amount of an agent that inhibits a signaling pathway of a receptor selected from the group consisting of epidermal growth factor receptor, platelet-derived growth factor receptor, ErbB2/Her2, progesterone receptor, and a combination thereof.

In another aspect, the invention provides a method of inhibiting invasion of an animal cell by a fungal cell comprising administering to the animal cell an effective amount of an agent that inhibits a signaling pathway of a receptor selected from the group consisting of epidermal growth factor receptor, platelet derived growth factor receptor, ErbB2/Her2, progesterone receptor, and a combination thereof.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. A teen who recovered from rhino-cerebral mucormycosis but was left with a facial defect.

FIG. 2. R. delemar invades alveolar epithelial cells via a mechanism that is independent of CotH3/GRP78 interactions. R. delemar transformed with empty plasmid or RNAi plasmid targeting CotH3 expression were incubated on confluent alveolar epithelial (A549) cells on coverslips for different time points after which the cells were fixed with 3% paraformaldehyde and stained with 1% Uvitex dye. The number of invading organisms was determined using differential fluorescence microscopy. N=6 slides per treatment from 2 independent experiments. Ten high power fields were counted per slide.

FIG. 3. R. delemar infection stimulates phosphorylation of PDGFR subunits. A549 alveolar epithelial cells were infected with R. delemar (strain 99-880) for the indicated times and phosphorylation of PDGFR subunits in whole cell lysates was assessed by sandwich ELISA. The capture antibodies were against α and β subunits of PDGFR. An anti-phosphotyrosine antibody served as the detection antibody. Values represent the fold-change observed at 450 nm. *; p<0.01. **; p<0.0001.

FIG. 4. EGFR becomes phosphorylated upon infection with R. delemar. (A) Immunoblot of whole cell lysates from Mucorales-infected A549 alveolar epithelial cells 3 hrs post-infection. Blots were probed with Abs against phosphorylated EGFR (pY0168) and total EGFR (EGFR). (B) Quantitation of the immunoblot from panel A. Values are the fold induction of the ratio of pEGFR to total EGFR.

FIG. 5. ErbB2 gets cleaved upon infection with Rhizopus. Immunoblot of whole cell lysates from Rhizopus-infected A549 alveolar epithelial cells. Left panel depict samples from R. delemar infection. Right panel depicts R. oryzae infection. Blots were probed with Abs against a C-terminal epitope (intracellular) of ErbB2. FL; Full length ErbB2 protein.

FIG. 6. Inhibition of receptor tyrosine kinase signaling inhibits Rhizopus-induced damage. A549 cells were pre-treated with a PDGFR inhibitor (Inh III, 10 an EGFR inhibitor (AG1478, 10 μM) or an ErbB2 inhibitor (AG825, 50 μM) or combinations and then infected with R. delemar. For all experiments, cells were pre-treated with inhibitors, vehicle alone, or combinations for 1 h and the inhibitors were also present throughout the course of infection. Cellular damage was assessed using LDH release assay after 24 h of infection. Results are the mean±SD. **P<0.01.*P<0.05. n=6-9 from 2 independent experiments.

FIG. 7. The EGFR inhibitor, Gefitinib, protects mice from pulmonary mucormycosis following delayed administration and is additive with antifungal treatment. Survival of neutropenic mice (n=15/group) infected intratracheally with R. delemar and treated with vehicle control (placebo), 10 mg/kg gefitinib (Gefitinib), 20 mg/kg posaconazole (Posaconazole) or both (Combo) beginning 30 h post infection for 6 consecutive days. *P0.0341, **P=0.0005 by Log Rank test compared to placebo.

FIG. 8. Working Model of growth factor receptor (GFR)-mediated invasion of airway epithelial cells by Mucorales. An inhaled spore binds to airway epithelial cells and germinates. Fungal proteins expressed on the hyphae interact with and activate GFRs which in turn activates a kinase signaling cascade resulting in endocytosis of the fungal cells and subsequent damage to the epithelial cell barrier.

FIG. 9. Adaptation of CRISP/Cas9 for gene disruption in R. delemar. (A) R. delemar pyrF mutant was transformed with a plasmid harboring the Cas9 nuclease complexed with a gRNA targeting a toxin-like gene causing a deletion of ˜100 bp (i.e. CRISPR 2.1 Southern blot of 1.9 kb vs. wild-type and empty plasmid [Pdc-Cas9] of 2.0 kb). The gene deletion was also confirmed by DNA sequencing. (B) qRT-PCR confirming abrogated expression of targeted gene.

FIG. 10. Diagram of convergence of Estrogen, Progesterone, and EGFR signaling pathways. Credit: Anastasia Kariagina, J. X. and S. Z. H. Jeffrey R. Leipprandt, Amphiregulin Mediates Estrogen, Progesterone, and EGFR Signaling in the Normal Rat Mammary Gland and in Hormone-Dependent Rat Mammary Cancers. HORM CANC, 2010: p. 229-244.

FIG. 11. Analysis of host regulatory pathways. (A) Experimental design for in vitro model of mucormycosis. (B) Heat map showing predicted activation or repression of known pathways during in vitro infection of airway epithelial cells with Mucorales strains. Predicted activation and predicted repression are represented. Arrows highlight the pathways focused on due to prediction of host receptor pathway activation. Panel (B) is adapted from Chibucos, et al. An integrated genomic and transcriptomic survey of mucormycosis-causing fungi. Nature Communications 2016.

FIG. 12. Inhibition of progesterone receptor and EGFR signaling inhibits Rhizopus-induced damage. A549 cells were pre-treated with a PGR inhibitor (Mifepristone, Mif, 25 or an EGFR inhibitor (Gefitinib, Gef, 25 or combination followed by R. delemar infection. For all experiments, cells were pre-treated 1 h with inhibitors, vehicle alone or combination, and the inhibitors were also present during the course of infection. Cellular damage was assessed using the LDH release assay after 24 h of infection. Data are expressed as median±interquartile range. **P<0.005 and #P=0.041 versus control by Wilcoxon rank-sum test. n=at least 6 from at least 2 independent experiments. Veh for Mif (Ethanol); Veh for Gef (DMSO).

FIG. 13. Inhibition of PGR and EGFR signaling inhibits R. delemar internalization. A549 alveolar epithelial cells were pre-treated overnight with 25 μM Mifepristone, Mif, or with 25 μM Gefitinib, Gef, for 1 h followed by 3 h infection with 2×10⁶ R. delemar spores. *P<0.0001 versus control by Wilcoxon rank-sum test. Data are expressed as median±interquartile range and represents at least two independent experiments done in at least duplicates counting at least 15 fields per condition. Veh for Mif (Ethanol); Veh for Gef (DMSO).

FIG. 14. Effects of PGR inhibition on Mucorales-induced host cell damage and invasion. A549 alveolar epithelial cells were pre-treated overnight with 25 μM Mifepristone, Mif, and/or 25 μM Gefitinib, Gif, for 1 h followed by 24 h infection with 2×10⁶ a) R. oryzae, b) M. circinelloides, c) L. corymbifera, or d) C. bertholletiae spores that were germinated for 1 h. *P<0.01 or **P<0.05 versus control by Wilcoxon rank-sum test. n=6 wells per group from at least 2 independent experiments. Data are expressed as median±interquartile range.

FIG. 15. EGF treatment has no effect on Rhizopus-induced damage. A549 cells were pre-treated with EGF or vehicle followed by R. delemar infection. EGF was also present during the course of infection. Cellular damage was assessed using the LDH release assay after 24 h of infection. Data are expressed as median±interquartile range. Veh versus treatment by Wilcoxon rank-sum test. n=at least 6 from at least 2 independent experiments.

FIG. 16. PGR translocation to the nucleus during R. delemar infection is partially blocked by Gefitnib. A549 cells were pretreated with Gefitinib for 1 h followed by infection with R. delemar for 2 h, fixed, and probed with antibody specific for human PGR and viewed by confocal microscopy. Data is representative of 2 independent experiments done in duplicate.

FIG. 17. Rhizopus-induced PGR translocation to the nucleus is species specific. A549 cells were infected with R. delemar for 2 h, fixed, and probed with abs specific for human PGR and pPGR, and viewed by confocal microscopy. Data is representative of 1 experiment done in duplicate. 99-880 is a R. delemar strain, and 99-892 is a R. oryzae strain.

FIG. 18. PGR antibodies recognize R. delemar protein. Western blots from whole cell lysates from A459 cells alone (H) or from R. delemar resting spores (S) or germlings (G) alone were probed with different PGR antibodies.

FIG. 19. Host response to R. delemar infection in vivo and in vitro. (A) Mouse upstream regulators that are predicted to be changed in R. delemar-infected lungs in a mouse DKA model of mucormycosis. Red indicates predicted activation (z score >2). Teal indicates predicted repression (z score <−2). Black indicates now predicted effect. (B) Expression of known mouse EGFR targets in lungs 14 hours post inoculation of in vivo DKA model. (C) Expression of known human EGFR targets in A549 cells at 6 and 16 hours following inoculation of an in vitro infection. For panels B and C, plotted are the log transformed RPKM values that have been normalized across all samples. Red indicates high gene expression; blue indicates low expression. Each column in panels B and C represents an individual sample from a different mouse.

FIG. 20. Localization of EGFR in A549 cells infected with R. delemar. A549 cells were infected for 30 minutes with 2×10⁵ R. delemar spores that had been germinated 1 h. Cells were then stained for pEGFR and EGFR.

FIG. 21. Effects of EGFR inhibition on invasion of airway epithelial cells by R. delemar. A549 alveolar epithelial cells were pre-treated with vehicle, 25 μM Gefitinib or 25 μg/ml or Cetuximab for 1 h followed by: (A, C) 3 h infection in the presence of inhibitor with 2×10⁵ R. delemar spores or (B, D) 24 h infection (in the presence of inhibitors) with 2×10⁶ R. delemar spores that were germinated for 1 h. *P<0.0001 or P<0.01 versus control by Wilcoxon rank-sum test. Data are expressed as median±interquartile range. Data represents at least two independent experiments.

FIG. 22. The EGFR inhibitor, Gefitinib, protects mice from pulmonary mucormycosis. (A) Survival of neutropenic mice (n=20/group from two independent experiments with similar results) infected intratracheally with R. delemar (average inoculum of 3.8×10³ spores per mouse) and treated with vehicle control (placebo) or 10 mg/kg Gefitinib 4 h post infection for 5 consecutive days. *P=0.0084 vs. placebo-treated mice by Log Rank test. (B) Tissue fungal burden of lungs and brains of mice (n=10 per group) infected intratracheally with R. delemar (5.6×10³ spores per mouse of confirmed inoculum) and treated with vehicle control (placebo) or Gefitinib. Data are presented as median+interquartile range.

FIG. 23. Gefitinib Inhibits in vitro infection of other Mucormycosis-causing fungi.

FIG. 24. Trastuzumab inhibits Rhizopus infection in vitro.

### DETAILED DESCRIPTION OF THE INVENTION

Mucormycosis is an increasingly common, highly lethal fungal infection with very limited treatment options. Using a combination of in vivo animal models, transcriptiomics, cell biology and pharmacological approaches, it is provided herein that Rhizopus arrhizus, the most common cause of mucormycosis, activates various signaling pathways such as the epidermal growth factor receptor, platelet-derived growth factor receptor, ErbB2/Her2, and progesterone receptor pathway to induce fungal uptake into airway epithelial cells.

As described herein in more detail, inhibition of epidermal growth factor receptor signaling pathways, for example, using an existing drug approved by the United States Food and Drug Administration, significantly increased survival following R. arrhizus infection in mice. The experiments described herein enhance an understanding of how Mucoralean fungi invade host cells during the establishment of pulmonary mucormycosis and demonstrate that EGFR as well as other signaling pathways are targets for inhibiting mucormycosis. For example, drugs that target EGFR function, such as cetuximab and gefitinib can be used alone or in combination together or with other drugs to treat or prevent mucormycosis.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and therapeutics, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3rd ed., 2002); Dictionary of Chemistry, Hunt (Ed.), Routledge (1′ ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

In one embodiment, the invention provides a method of treating or preventing mucormycosis in a subject in need thereof, comprising administering to the subject a therapeutically-effective amount of an agent that inhibits a signaling pathway of a receptor selected from the group consisting of epidermal growth factor receptor, platelet-derived growth factor receptor, ErbB2/Her2, progesterone receptor and a combination thereof.

In another embodiment, the invention provides a method of inhibiting invasion of an animal cell by a fungal cell comprising administering to the animal cell an effective amount of an agent that inhibits a signaling pathway of a receptor selected from the group consisting of epidermal growth factor receptor, platelet-derived growth factor receptor, ErbB2/Her2, progesterone receptor and a combination thereof. In one embodiment, the fungal cell is a cell of the order Mucorales. In some embodiments, the animal cell is a mammalian cell or an avian cell. In some embodiments, the mammalian cell is a human cell. In some embodiments, the human cells are A549 human alveolar epithelial cells.

The term “subject” as used herein is not limiting and is used interchangeably with patient. In some embodiments, the subject refers to animals, such as birds, mammals and the like. For example, mammals contemplated include humans, primates, dogs, cats, sheep, cattle, goats, pigs, horses, chickens, mice, rats, rabbits, guinea pigs, and the like.

As used herein, the term “mucormycosis” is intended to mean a fungal condition caused by fungi of the order Mucorales. Mucormycosis is a life-threatening fungal infection almost uniformly affecting immunocompromised hosts in either developing or industrialized countries. Fungi belonging to the order Mucorales are distributed into at least six families, all of which can cause cutaneous and deep infections. Species belonging to the family Mucoraceae are isolated more frequently from patients with mucormycosis than any other family. Among the Mucoraceae, Rhizopus oryzae (Rhizopus arrhizus) is a common cause of infection. Other exemplary species of the Mucoraceae family that cause a similar spectrum of infections include, for example, Rhizopus microsporus var. rhizopodiformis, Absidia corymbifera, Apophysomyces elegans, Mucor species, Rhizomucor pusillus and Cunninghamella spp (Cunninghamellaceae family). Mucormycosis is well known in the art and includes, for example, rinocerebral mucormycosis, pulmonary mucormycosis, gastrointestinal mucormycosis, disseminated mucormycosis, bone mucormycosis, mediastinum mucormycosis, trachea mucormycosis, kidney mucormycosis, peritoneum mucormycosis, superior vena cava mucormycosis or external otitis mucormycosis.

Fungi belonging to the order Mucorales are currently distributed into the families of Choanephoraceae; Cunninghamellaceae; Mucoraceae; Mycotyphaceae; Phycomycetaceae; Pilobolaceae; Saksenaeaceae; Syncephalastraceae; and Umbelopsidaceae. Each of these fungi families consist of one or more genera. For example, fungi belonging to the order Mucorales, family Mucoraceae, are further classified into the genera of Absidia (e.g., A. corymbifera); Actinomucor (e.g., A. elegans); Amylomyces (e.g., A. rouxii); Apophysomyces; Backusella (e.g., B. circina); Benjaminiella (e.g., B. multispora); Chaetocladium (e.g., C. brefeldii); Circinella (e.g., C. angarensis); Cokeromyces (e.g., C. recurvatus); Dicranophora (e.g., D. fulva); Ellisomyces (e.g., E. anomalus; Helicostylum (e.g., H. elegans); Hyphomucor (e.g., H. assamensis); Kirkomyces (e.g., K. cordensis); Mucor (e.g., M. amphibiorum); Parasitella (e.g., P. parasitica); Philophora (e.g., P. agaricina); Pilaira (e.g., P. anomala); Pirella (e.g., P. circinans); Rhizomucor (e.g., R. endophyticus); Rhizopodopsis (e.g., R. javensis); Rhizopus; Sporodiniella (e.g., S. umbellata); Syzygites (e.g., S. megalocarpus); Thamnidium (e.g., T. elegans); Thermomucor (e.g., T. indicae-seudaticae); and Zygorhynchus (e.g., Z. californiensis). The genus Rhizopus, for example, consists of R. azygosporus; R. caespitosus; R. homothallicus; R. oryzae; and R. schipperae species.

The Choanephoraceae family consists of fungi genera Blakeslea (e.g., B. monospora), Choanephora (e.g., C. cucurbitarum), Gilbertella (e.g., G. hainanensis), and Poitrasia (e.g., P. circinans). The Cunninghamellaceae family consists of genera Chlamydoabsidia (e.g., C. padenii); Cunninghamella (e.g., C. antarctica); Gongronella (e.g., G. butleri); Halteromyces (e.g., H. radiatus); and Hesseltinella (e.g., H. vesiculosa). The Mycotyphaceae family consists of fungi genus Mycotypha (e.g., M. africana). The Phycomycetaceae family consists of fungi genus Phycomyces (e.g., P. blakesleeanus) and Spinellus (e.g., S. chalybeus). The Pilobolaceae family consists of fungi genera Pilobolus (e.g., P. longipes) and Utharomyces (e.g., U. epallocaulus). The Saksenaeaceae family consists of fungi genera Apophysomyces (e.g., A. elegans) and Saksenaea (e.g., S. vasiformis). The Syncephalastraceae family consists of fungi genera Dichotomocladium (e.g., D. elegans); Fennellomyces (e.g., F. gigacellularis); Mycocladus (e.g., M. blakesleeanus); Phascolomyces (e.g., P. articulosus); Protomycocladus (e.g., P. faisalabadensis); Syncephalastrum (e.g., S. monosporum); Thamnostylum (e.g., T. lucknowense); Zychaea (e.g., Z. mexicana). Finally, the Umbelopsidaceae family consists of fungi genus Umbelopsis (e.g., U. angularis).

The term “treating” or “treatment,” as it is used herein is intended to mean an amelioration of a clinical symptom indicative of mucormycosis. Amelioration of a clinical symptom includes, for example, a decrease or reduction in at least one symptom of mucormycosis in a treated individual compared to pretreatment levels or compared to an individual with mucormycosis. The term “treating” also is intended to include the reduction in severity of a pathological condition, a chronic complication or an opportunistic fungal infection which is associated with mucormycosis. Mucormycosis can be found described in, for example, Merck Manual, Sixteenth Edition, 1992, and Spellberg et al., Clin. Microbio. Rev. 18:556-69 (2005).

The term “preventing” or “prevention,” as it is used herein is intended to mean a forestalling of a clinical symptom indicative of mucormycosis. Such forestalling can include, for example, the maintenance of normal physiological indicators in an individual at risk of infection by a fungus or fungi prior to the development of overt symptoms of the condition or prior to diagnosis of the condition. Therefore, the term “preventing” includes the prophylactic treatment of individuals to guard them from the occurrence of mucormycosis. Preventing mucormycosis in an individual also is intended to include at least partially inhibiting or arresting the development of mucormycosis. Inhibiting or arresting the development of the condition includes, for example, inhibiting or arresting the occurrence of abnormal physiological indicators or clinical symptoms. Therefore, effective prevention of mucormycosis could include maintenance of normal body temperature, weight, psychological state as well as lack of lesions or other pathological manifestations in an individual predisposed to mucormycosis. In some embodiments, an individual predisposed or at risk of mucormycosis could include, for example, an individual with AIDS, azotemia, diabetes mellitus, bronchiectasis, emphysema, TB, lymphoma, leukemia, or burns, or an individual with a history of susceptibility to a fungal condition. In some embodiments, the subject at risk of infection has uncontrolled diabetes mellitus that results in hyperglycemia and ketoacidosis (DKA), other forms of acidosis, undergone treatment with corticosteroids, undergone solid organ, bone marrow or stem cell transplant, has neutropenia (low number of white blood cells), has a history of injection drug use, elevated iron levels (iron overload or hemochromatosis), skin injury due to surgery, trauma, burns, or wounds (e.g., wounded soldiers), malignant haematological disorders, undergone deferoxamine therapy and receiving haemodialysis, or being premature and having low birthweight (for neonatal gastrointestinal mucormycosis).

In some embodiments, the methods of the invention treat mucormycosis in the subject. In some embodiments, the methods of the invention prevent mucormycosis in the subject.

In accordance with the invention, a “therapeutically effective amount” or “effective amount” of the agent is administered to the subject. As used herein a “therapeutically effective amount” or “effective amount” is an amount sufficient to prevent, reduce or attenuate mucormycosis in the subject or patient.

The therapeutically active agent that inhibits signaling by the epidermal growth factor receptor, platelet-derived growth factor receptor, ErbB2/Her2, or progesterone receptor is not limiting and can include, for example, a small molecule (e.g., <1000 Da), nucleic acid, carbohydrate, lipid, peptide, polypeptide, protein or antibody or active antibody fragment.

Therapeutically active agents can be identified by various methods, including library screening, phage display, antibody generation, and antibody screening. In one embodiment, the agent is an antibody or an active fragment thereof. In some embodiments, the therapeutically active agent can be identified or screened on the basis of its ability to inhibit invasion of an animal cell by a fungal cell by contacting the cell with the therapeutically active agent and assaying for inhibition of invasion by the fungal cell. Known agents, including agents that have achieved regulatory approval for the treatment of other conditions and that are capable of inhibiting signaling by the epidermal growth factor receptor, platelet-derived growth factor receptor, ErbB2/Her2, or progesterone receptor can also be employed in the methods of the invention. Combinations of such agents can also be used, as well as combinations with existing treatments for treating or preventing mucormycosis, such as one or more antifungal agents or surgery.

In some embodiments, the therapeutic agent is an antibody or an active fragment thereof. In some embodiments, the antibody can be a monoclonal, polyclonal, human, humanized or non-human antibody. The term “antibody” means an immunoglobulin molecule that recognizes and binds to a target through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments, dual affinity retargeting antibodies (DART)), single chain Fv (scFv) mutants, multispecific antibodies such as bispecific and trispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

In some embodiments, an antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The basic four-chain antibody unit is a heterotetrameric glycoprotein composed of two identical light (L) chains and two identical heavy (H) chains. Each H chain has at the N-terminus, a variable region (V_{H}) followed by three constant domains (C_{H}) for each of the α and γ chains and four C_{H} domains for μ and ε isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ) respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “active antibody fragment” or antibody fragment refers to a portion of an intact antibody and comprises the antigenic determining variable regions of an intact antibody. Examples of active antibody fragments include, but are not limited to Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g. murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g. mouse, rat, rabbit, hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)).

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

An “intact” antibody is one that comprises an antigen-binding site as well as a C_{L} and at least heavy chain constant domains, C_{H1}, C_{H2} and C_{H3}. The constant domains may be native sequence constant domains (e.g., human native sequence constant domains) or amino acid sequence variants thereof.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc) with the desired specificity, affinity, and capability while the constant regions are homologous to the sequences in antibodies derived from another (usually human) to avoid eliciting an immune response in that species.

The antibodies that can be used herein also include antibodies in which a portion of the heavy and/or light chain is identical with or homologous to corresponding sequences in antibodies belonging to a particular antibody class or subclass, while the remainder of the chain(s) is identical with or homologous to corresponding sequences in antibodies derived from another species or belonging to another antibody class or subclass, as well as fragments of such antibodies.

In some embodiments, chimeric antibodies may comprise residues that are not found in the recipient antibody or in the donor antibody. In some embodiments, modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

### Epidermal Growth Factor Receptor Inhibitors

The human epidermal growth factor receptor (also known as HER-1 or Erb-B1, and referred to herein as “EGFR”) is a 170 kDa transmembrane receptor encoded by the c-erbB protooncogene, and exhibits intrinsic tyrosine kinase activity (Modjtahedi et al., Br. J. Cancer 73:228-235 (1996); Herbst and Shin, Cancer 94:1593-1611 (2002)). SwissProt database entry P00533 provides the sequence of human EGFR. EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay et al., Ann. Oncology 14:1346-1363 (2003); Tsao and Herbst, Signal 4:4-9 (2003); Herbst and Shin, Cancer 94:1593-1611 (2002); Modjtahedi et al., Br. J. Cancer 73:228-235 (1996)).

Known ligands of EGFR include EGF, TGFA/TGF-alpha, amphiregulin, epigen/EPGN, BTC/betacellulin, epiregulin/EREG and HBEGF/heparin-binding EGF. Ligand binding by EGFR triggers receptor homo- and/or heterodimerization and autophosphorylation of key cytoplasmic residues. The phosphorylated EGFR recruits adapter proteins like GRB2 which in turn activate complex downstream signaling cascades, including at least the following major downstream signaling cascades: the RAS-RAF-MEK-ERK, PI3 kinase-AKT, PLCγ-PKC, and STATs modules. This autophosphorylation also elicits downstream activation and signaling by several other proteins that associate with the phosphorylated tyrosines through their own phosphotyrosine-binding SH2 domains. These downstream signaling proteins initiate several signal transduction cascades, principally the MAPK, Akt and JNK pathways, leading to cell proliferation. Ligand binding by EGFR may also activate the NF-kappa-B signaling cascade. Ligand binding also directly phosphorylates other proteins like RGS16, activating its GTPase activity and potentially coupling the EGF receptor signaling to G protein-coupled receptor signaling. Ligand binding also phosphorylates MUC1 and increases its interaction with SRC and CTNNB 1/beta-catenin.

EGFR is found at abnormally high levels in cancer cells, and EGFR activation appears to be important in tumor growth and progression. Some types of cancers show mutations in their EGFRs, which may cause unregulated cell division through continual or abnormal activation of the EGFR. There are many known drugs that can be useful to inhibit EGFR. These drugs can include tyrosine kinase inhibitors (TKI) (e.g., erlotinib, gefitinib) that bind to the tyrosine kinase domain in the epidermal growth factor receptor and stop the activity of the EGFR and antibodies (e.g. cetuximab, necitumumab) which bind to the extracellular component of the EGFR and prevent epidermal growth factor from binding to its own receptor, therefore preventing cell division. EGFR inhibitors are used in the treatment of cancers that are caused by EGFR up-regulation, such as non-small-cell lung cancer, pancreatic cancer, breast cancer, and colon cancer.

In some embodiments, the therapeutically active agent of the invention inhibits the signaling pathway of the epidermal growth factor receptor. In some embodiments, the therapeutically active agent binds to the epidermal growth factor receptor and inhibits its activity. In some embodiments, the therapeutically active agent inhibits a tyrosine kinase activity. In some embodiments, the therapeutically active agent inhibits a signaling molecule downstream of the epidermal growth factor receptor.

In some embodiments, the therapeutically active agent inhibits a tyrosine kinase activity of epidermal growth factor receptor. There are many known tyrosine kinase inhibitors to the epidermal growth factor receptor, and such inhibitors can be used in the methods of the invention. In some embodiments, the agent is selected from AG1478, gefitinib, GW2974 erlotinib, neratinib, osimertinib, vandetanib, dacomitinib and a combination thereof.

In some embodiments, the agent is an antibody or an active fragment thereof that binds to the epidermal growth factor receptor. There are many known antibodies to the epidermal growth factor receptor, and such antibodies can be used in the methods of the invention. In some embodiments, the antibody is selected from cetuximab, panitumumab, necitumumab and a combination thereof.

In some embodiments, the agent targets the ATP binding pocket of both EGFR and ErbB2. In some embodiments, the agent is lapatinib (Tykerb).

### Platelet-Derived Growth Factor Receptor Inhibitors

Platelet-derived growth factors (PDGFs) are potent mitogens that exist as five different dimeric configurations composed of four different isoform subunits: A, B, C and D. The five dimeric forms of the PDGFs are AA, BB, AB, CC and DD, which are formed by disulfide linkage of the corresponding individual PDGF monomers. PDGF ligands exert their biological effects through their interactions with PDGF receptors (PDGFRs). PDGFRs are single-pass, transmembrane, tyrosine kinase receptors composed of heterodimeric or homodimeric associations of an alpha (a) receptor chain (PDGFR-alpha) and/or a beta (β) receptor chain (PDGFR-beta). Thus, active PDGFRs may consist of αα, ββ, or αβ receptor chain pairings. PDGFRs share a common domain structure, including five extracellular immunoglobulin (Ig) loops, a transmembrane domain, and a split intracellular tyrosine kinase (TK) domain. The interaction between dimeric PDGF ligands and PDGFRs leads to receptor chain dimerization, receptor autophosphorylation and intracellular signal transduction. It has been demonstrated in vitro that ββ receptors are activated by PDGF-BB and -DD, while αβ receptors are activated by PDGF-BB, —CC, -DD and -AB, and aa receptors are activated by PDGF-AA, —BB, —CC and -AB (see Andrae et al. (2008) Genes Dev 22 (10):1276-1312).

PDGF signaling has been implicated in various human diseases including diseases associated with pathological neovascularization, vascular and fibrotic diseases, tumor growth and eye diseases.

In some embodiments, the therapeutically active agent inhibits the signaling pathway of the platelet-derived growth factor receptor. In some embodiments, the therapeutically active agent binds to the platelet-derived growth factor receptor and inhibits its activity. In some embodiments, the therapeutically active agent inhibits a signaling molecule downstream of the platelet-derived growth factor receptor.

In some embodiments, the therapeutically active agent inhibits a tyrosine kinase activity of platelet-derived growth factor receptor. There are many known tyrosine kinase inhibitors to the platelet-derived growth factor receptor, and such inhibitors can be used in the methods of the invention. In some embodiments, the agent is selected from CAS 205254-94-0, imatinib (Gleevec/STI-571), CP-673451, sunitinib, sorafenib, pazopanib, nilotinib, cediranib, motesanib, axitinib, linifenib, dasatinib, quizartinib, ponatinib and combinations thereof.

In some embodiments, the agent is an antibody or an active fragment thereof that binds to the platelet-derived growth factor receptor. There are many known antibodies to the platelet-derived growth factor receptor, and such antibodies can be used in the methods of the invention. See, e.g., U.S. Pat. Nos. 7,060,271; 5,882,644; 7,740,850; and U.S. Patent Appl. Publ. No. 2011/0177074, which are incorporated by reference herein.

### ErbB2/Her2 Inhibitors

ErbB2, also known as v-erb-b2 avian erythroblastic leukemia viral oncogene homolog 2, Her2, Neu, CD340, proto-oncogene C-ErbB2, is a member of the extensively studied ErbB family of plasma membrane-bound receptor tyrosine kinases, which also includes ErbB1, ErbB3 and ErbB4 (also known as EGFR/Her1, Her3 and Her4, respectively). These receptors have been shown to play critical roles in embryonic development, normal physiology and the development of various diseases. All four ErbB receptors contain an extracellular domain (ECD), a transmembrane domain and an intracellular domain that interacts with signaling molecules. Ligand binding to the ECDs of these receptors leads to homo- or hetero-dimerization, followed by the activation of the intrinsic protein tyrosine kinase and tyrosine autophosphorylation in the intracellular domain, and recruitment and activation of signaling proteins to these sites.

ErbB2 is best known for its involvement in human breast cancer. ErbB2 gene amplification occurs in 20-30% of breast cancer and is significantly correlated with ErbB2 protein expression in the cancer tissues. ErbB2-targeted therapies, particularly humanized monoclonal antibody trastuzumab in combination with chemotherapy, show considerable clinical efficacy.

In some embodiments, the therapeutically active agent inhibits the signaling pathway of ErbB2/Her2. In some embodiments, the therapeutically active agent binds to ErB2/Her2 and inhibits its activity. In some embodiments, the therapeutically active agent inhibits a signaling molecule downstream of ErB2/Her2.

In some embodiments, the therapeutically active agent inhibits tyrosine kinase activity of ErbB2/Her2. There are many known tyrosine kinase inhibitors to ErbB2/Her2, and such inhibitors can be used in the methods of the invention. In some embodiments, the therapeutically active agent is selected from compound AG825, compound GW2974, neratinib, dacomitinib and combinations thereof.

In some embodiments, the agent is an antibody or an active fragment thereof that binds to ErbB2/Her2. There are many known antibodies to ErbB2/Her2, and such antibodies can be used in the methods of the invention. In some embodiments, the antibody is selected from trastuzumab, pertuzumab and combinations thereof.

In some embodiments, the agent is an inhibitor that targets the ATP binding pocket of both EGFR and ErbB2. In some embodiments, the agent is lapatinib (Tykerb).

### Progesterone Receptor Inhibitors

Steroid receptors include the progesterone receptor (PR), androgen receptor (AR), estrogen receptor (ER), glucocorticoid receptor (GR) and mineralocorticoid receptor (MR). Regulation of a gene by such factors requires the receptor and a corresponding ligand which has the ability to selectively bind to the receptor in a way that affects gene transcription.

Progesterone receptor modulators (progestagens) are known to play an important role in the health of women. The natural ligand for the progesterone receptor is the steroid hormone progesterone, but synthetic compounds have been made which may also serve as ligands (see, e.g., Jones et al., U.S. Pat. No. 5,688,810).

In some embodiments, the therapeutically active agent inhibits the signaling pathway of the progesterone receptor. In some embodiments, the agent is selected from mifepristone, aglepristone, ulipristal and combinations thereof. See, e.g., Spitz, Progesterone antagonists and progesterone receptor modulators, Expert Opin Investig Drugs, 12(10):1693-707 (2003), and U.S. Pat. Nos. 9,109,004 and 8,053,426 which are incorporated by reference herein.

### Combination Therapy

In some embodiments, a combination of therapeutic agents are administered to the subject. In some embodiments, the combination of therapeutic agents can be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In some embodiments, a combination of agents that inhibit the epidermal growth factor signaling pathway are administered. In some embodiments, the subject is administered cetuximab in combination with gefitinib.

In some embodiments, a combination of agents that inhibit the platelet-derived growth factor signaling pathway are administered.

In some embodiments, a combination of agents that inhibit the ErbB2/Her2 signaling pathway are administered.

In some embodiments, a combination of agents that inhibit the progesterone receptor signaling pathway are administered.

In some embodiments, one or more agents that inhibit the signaling pathway of the epidermal growth factor receptor is administered in combination with one or more agents that inhibit the signaling pathway of the platelet-derived growth factor receptor. In some embodiments, the subject is administered AG1478, gefitinib, GW2974, erlotinib, neratinib, osimertinib, vandetanib, dacomitinib, cetuximab, panitumumab, necitumumab, and/or lapatinib in combination with CAS 205254-94-0, imatinib (Gleevec/STI-571), CP-673451, sunitinib, sorafenib, pazopanib, nilotinib, cediranib, motesanib, axitinib, linifenib, dasatinib, quizartinib, and/or ponatinib.

In some embodiments, one or more agents that inhibit the signaling pathway of the epidermal growth factor receptor is administered in combination with one or more agents that inhibit the signaling pathway of ErbB2/Her2. In some embodiments, the subject is administered AG1478, gefitinib, GW2974 erlotinib, neratinib, osimertinib, vandetanib, dacomitinib, cetuximab, panitumumab, necitumumab and/or lapatinib in combination with compound AG825, compound GW2974, neratinib, dacomitinib, trastuzumab, pertuzumab and/or lapatinib.

In some embodiments, one or more agents that inhibit the signaling pathway of the epidermal growth factor receptor is administered in combination with one or more agents that inhibit the signaling pathway of the progesterone receptor. In some embodiments, the subject is administered AG1478, gefitinib, GW2974 erlotinib, neratinib, osimertinib, vandetanib, dacomitinib, cetuximab, panitumumab, necitumumab and/or lapatinib in combination with mifepristone, aglepristone and/or ulipristal.

In some embodiments, one or more agents that inhibit the signaling pathway of the platelet-derived growth factor receptor is administered in combination with one or more agents that inhibit the signaling pathway of ErbB2/Her2. In some embodiments, the subject is administered CAS 205254-94-0, imatinib (Gleevec/STI-571), CP-673451, sunitinib, sorafenib, pazopanib, nilotinib, cediranib, motesanib, axitinib, linifenib, dasatinib, quizartinib, and/or ponatinib in combination with compound AG825, compound GW2974, neratinib, dacomitinib, trastuzumab, pertuzumab and/or lapatinib.

In some embodiments, one or more agents that inhibit the signaling pathway of the progesterone receptor is administered in combination with one or more agents that inhibit the signaling pathway of ErbB2/Her2. In some embodiments, the subject is administered mifepristone, aglepristone and/or ulipristal in combination with compound AG825, compound GW2974, neratinib, dacomitinib, trastuzumab, pertuzumab and/or lapatinib.

In some embodiments, one or more agents that inhibit the signaling pathway of the platelet-derived growth factor receptor is administered in combination with one or more agents that inhibit the signaling pathway of the progesterone receptor. In some embodiments, the subject is administered CAS 205254-94-0, imatinib (Gleevec/STI-571), CP-673451, sunitinib, sorafenib, pazopanib, nilotinib, cediranib, motesanib, axitinib, linifenib, dasatinib, quizartinib, and/or ponatinib in combination with mifepristone, aglepristone and/or ulipristal.

In some embodiments, the therapeutic agent or combination of agents as described herein can be administered in further combination with one or more methods or compositions available for fungal therapy.

In some embodiments, one or more antifungal agents are administered. In some embodiments, the antifungal agent is selected from the group consisting of amphotericin B (AmB), isavuconazole, posaconazole, fluconazole, itraconazole, ketoconazole, iron chelators such as, for example, deferasirox, or deferiprone.

In some embodiments, the therapeutic agents or combination of agents of the invention can be used in concert with a surgical method to treat a fungal infection. In another embodiment, the therapeutic agents or combination of agents of the invention can be used in combination radiation therapy for treating a fungal condition. Radiations useful in combination therapies for treating fungal infections include electromagnetic radiations such as, for example, near infrared radiation with specific wavelength and energy useful for treating fungal infections.

### Compositions

In another embodiment, the invention provides compositions for use in the methods of the invention comprising one or more therapeutically active agents as described herein.

In another embodiment, the invention provides for the use of a therapeutically active agent for the manufacture of a medicament for the treatment or prevention of mucormycosis.

The amount of the therapeutic agents of the invention which will be effective in the treatment or prevention of mucormycosis can be determined by standard clinical techniques. In addition, in vitro assays can optionally be employed to help identify optimal dosage ranges. The precise dose to be employed in the formulation will also depend on the route of administration, and the seriousness of the disease or condition, and should be decided according to the judgment of the practitioner and each subject's circumstances. Effective doses can be extrapolated from dose-response curves derived from in vitro or animal model test systems.

One skilled in the art can readily determine an appropriate dosage regimen for administering a therapeutically active agent of the invention to a given subject. For example, the compound(s) or composition(s) can be administered to the subject once, such as by a single injection or deposition at or near the site of interest. In some embodiments, the compound(s) or composition(s) can be administered to a subject once or twice daily to a subject, once weekly, every two weeks, etc. In some embodiments, the agents are administered for a period of from about three to about twenty-eight days, in some embodiments, from about seven to about ten weeks. In some dosage regimens, the compound(s) or composition(s) is injected at or near the site of interest. Where a dosage regimen comprises multiple administrations, it is understood that the effective amount of the compound(s) or composition(s) administered to the subject can comprise the total amount of the compound(s) or composition(s) administered over the entire dosage regimen. The exact amount will depend on the purpose of the treatment, the subject to be treated, and will be ascertainable by a person skilled in the art using known methods and techniques for determining effective doses. In some embodiments, the amount of the therapeutic agent that can be administered includes between about 0.1 μg/kg/day to about 100 mg/kg/day. In some embodiments, the amount of the therapeutic agent that can be administered includes between about 1.0 μg/kg/day to about 10 mg/kg/day. In some embodiments, the therapeutic agent is an antibody and the amount of antibody administered can be in the range of about 0.1 mg/kg to about 20 mg/kg of patient body weight, whether, for example, by one or more separate administrations, or by continuous infusion.

Various delivery systems are known and can be used to administer the therapeutic agents of the invention. The agents can be administered by any convenient route, for example by infusion or bolus injection, by absorption through epithelial or mucocutaneous linings (e.g., oral mucosa, rectal and intestinal mucosa, etc.) and can be administered together with other biologically active agents. Administration can be systemic or local.

The compositions can be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods. In some embodiments, the agent is administered orally, intranasally, intraocularly, intracerebroventricularly, intracerebrally, intrapulmonarily, intravenously, topically, subcutaneously, intradermally, and/or intramuscularly.

In a specific embodiment, it can be desirable to administer the therapeutic agents locally to the area in need of treatment; this can be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, care must be taken to use materials to which the protein does not absorb. In another embodiment, the agent can be delivered in liposomes. In yet another embodiment, the agent can be delivered in a controlled release system.

In some embodiments, the compositions are formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule indicating the quantity of active agent. Where the compositions are to be administered by infusion, they can be dispensed with an infusion bottle containing sterile pharmaceutical grade water or saline. Where the compositions are administered by injection, an ampoule of sterile water for injection or saline can be provided so that the ingredients can be mixed prior to administration.

The therapeutic agents can also be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In some embodiments, the compositions comprise one or more antibodies of the invention. In certain embodiments, the compositions are pharmaceutical compositions. In some embodiments, formulations are prepared for storage and use by combining an antibody with a pharmaceutically acceptable vehicle (e.g. carrier, excipient) (Remington, The Science and Practice of Pharmacy 20th Edition Mack Publishing, 2000). In some embodiments, pharmaceutical compositions of the present invention are characterized as being at least sterile and pyrogen-free. As used herein, “pharmaceutical formulations” include formulations for human and veterinary use. Pharmaceutical compositions of the invention can be packaged for use in liquid form, or can be lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosacchandes, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

Controlled-release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

Polymers can be used for ion-controlled release of antibody compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

In some embodiments, the composition is administered parenterally. Suitable parenteral administration routes include intravascular administration (e.g. intravenous bolus injection, intravenous infusion, intra-arterial bolus injection, intra-arterial infusion and catheter instillation into the vasculature); peri- and intra-tissue administration; subcutaneous injection or deposition including subcutaneous infusion (such as by osmotic pumps); direct (e.g., topical) application to the area at or near the site of interest, for example by a catheter or other placement device; and inhalation.

In some embodiments, the agent or combination is formulated for ocular delivery. In some embodiments, the agent or combination is administered in one or more compositions comprising eye drops. In some embodiments, the compositions can treat or prevent orbital mucormycosis.

The compositions can be administered in a single dose or in multiple doses. Where the administration of a composition is by infusion, the infusion can be a single sustained dose or can be delivered by multiple infusions.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. Inhibition of EGFR Signaling Protections Against Mucormycosis

RNA-seq analysis on a murine model of mucormycosis. We analyzed the host transcriptional response to R. delemar in a murine model of pulmonary mucormycosis. Diabetic ketoacidosis (DKA) was induced in four groups of 3 male ICR mice that were subsequently inoculated with 2.5×10⁵ R. delemar spores (strain 99-880) or an equivalent volume of phosphate buffered saline (negative control). At 14 or 24 hours post-inoculation, animals were sacrificed and lungs were harvested for extraction of total RNA for subsequent transcriptome analysis using RNA-seq. These time points were chosen because they represent early stages of infection, prior to the onset of massive tissue damage and necrosis which might complicate interpretation of transcriptome analyses. The early time points also allowed us to focus on the initial response of the lung tissue during adhesion of spores to and subsequent invasion of the airway epithelium. From each of the 12 sequencing libraries, we obtained an average of 96.4±15.8 million reads that mapped to the mouse reference genome. The inclusion of a poly (A) enrichment step in the RNA-seq protocol, as predicted, resulted in the detection of transcripts from the infecting fungus. However, a robust analysis of the R. delemar transcriptome was precluded by the lack of sufficient reads that mapped to the R. delemar reference genome (6,488 reads combined from all six infected samples). Therefore, we focused our analysis of these samples on the host response.

We have previously demonstrated that infection-induced transcriptome changes can be used to identify signaling pathways that govern the interaction between host and fungal pathogen (Bruno V M, Shetty A C, Yano J, Fidel P L, Jr., Noverr M C, Peters B M. 2015. Transcriptomic analysis of vulvovaginal candidiasis identifies a role for the NLRP3 inflammasome. MBio 6; Chibucos M C, Soliman S, Gebremariam T, Lee H, Daugherty S, Orvis J, Shetty A C, Crabtree J, Hazen T H, Etienne K A, Kumari P, O'Connor T D, Rasko D A, Filler S G, Fraser C M, Lockhart S R, Skory C D, Ibrahim A S, Bruno V M. 2016. An integrated genomic and transcriptomic survey of mucormycosis-causing fungi. Nat Commun 7:12218; Liu Y, Shetty A C, Schwartz J A, Bradford L L, Xu W, Phan Q T, Kumari P, Mahurkar A, Mitchell A P, Ravel J, Fraser C M, Filler S G, Bruno V M. 2015. New signaling pathways govern the host response to C. albicans infection in various niches. Genome Res 25:679-89). Using the Ingenuity Pathway Analysis software (Ingenuity systems; http://www.ingenuity.com), we performed an Upstream Regulator Analysis on the sets of host genes that were differentially expressed (p<0.05) between the infection groups and the appropriate time-matched negative control groups.

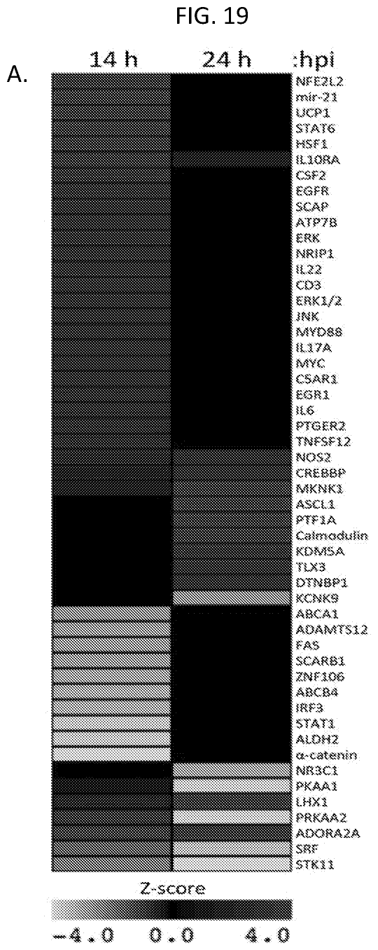

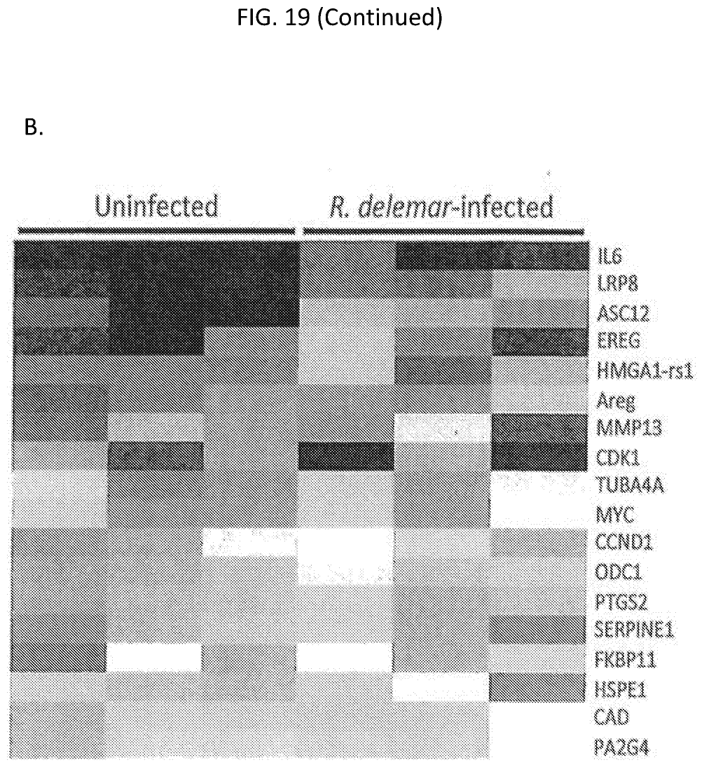

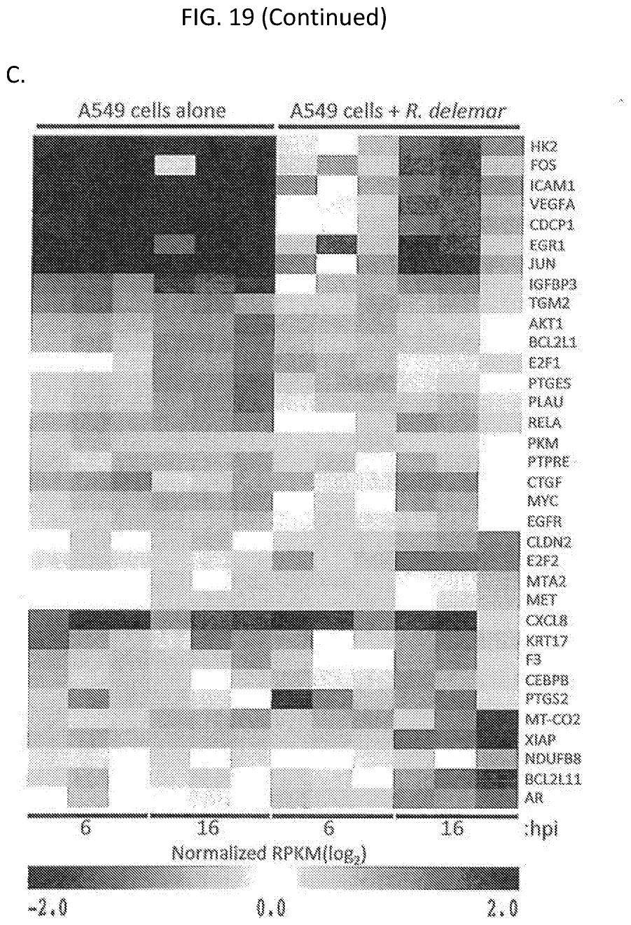

This approach was validated by our identification of two pathways that were already linked to infection by Mucorales: IL-22 (interleukin 22) and IL-17A (interleukin 17A) (FIG. 19A) (Bao W, Jin L, Fu H J, Shen Y N, Lu G X, Mei H, Cao X Z, Wang H S, Liu W D. 2013. Interleukin-22 mediates early host defense against Rhizomucor pusilluscan pathogens. PLoS One 8:e65065). Our analysis also predicted the activation of signaling pathways that have not been tied to mucormycosis but have been associated with the host response to fungal infection including: CSF2 (colony stimulating factor 2), ERK (extracellular signal-regulated kinases), MYD88 (Myeloid Differentiation Primary Response 88), and JNK (JUN N-Terminal Kinase) (Flemming A. 2017. Antifungals: INK inhibitors boost antifungal immunity. Nat Rev Drug Discov 16:163; Gavino C, Hamel N, Zeng J B, Legault C, Guiot M C, Chankowsky J, Lejtenyi D, Lemire M, Alarie I, Dufresne S, Boursiquot J N, McIntosh F, Langelier M, Behr M A, Sheppard D C, Foulkes W D, Vinh D C. 2016. Impaired RASGRF1/ERK-mediated GM-CSF response characterizes CARDS deficiency in French-Canadians. J Allergy Clin Immunol 137:1178-1188 e7; T, Medzhitov R. 2001. Drosophila MyD88 is an adapter in the Toll signaling pathway. Proc Natl Acad Sci USA 98:12654-8; Marr K A, Balajee S A, Hawn T R, Ozinsky A, Pham U, Akira S, Aderem A, Liles W C. 2003. Differential role of MyD88 in macrophage-mediated responses to opportunistic fungal pathogens. Infect Immun 71:5280-6; Moyes D L, Runglall M, Murciano C, Shen C, Nayar D, Thavaraj S, Kohli A, Islam A, Mora-Montes H, Challacombe S J, Naglik J R. 2010. A biphasic innate immune MAPK response discriminates between the yeast and hyphal forms of Candida albicans in epithelial cells. Cell host & microbe 8:225-35; Wang J, Gigliotti F, Bhagwat S P, Maggirwar S B, Wright T W. 2007. Pneumocystis stimulates MCP-1 production by alveolar epithelial cells through a JNK-dependent mechanism. Am J Physiol Lung Cell Mol Physiol 292:L1495-505; Wozniok I, Hornbach A, Schmitt C, Frosch M, Einsele H, Hube B, Loffler J, Kurzai O. 2008. Induction of ERK-kinase signalling triggers morphotype-specific killing of Candida albicans filaments by human neutrophils. Cell Microbiol 10:807-20.) (FIG. 19A). We also noticed a striking temporal dynamic in our dataset. Specifically, the majority of the pathways are modulated at 14 hours post-inoculation (h.p.i.) and returning to normal 24 h post infection. Furthermore, a smaller subset of pathways were modulated only at 24 h post infection.

Of particular interest was the significant overlap between genes that are differentially expressed 14 hours post inoculation with R. delemar and the known transcriptional targets of the epidermal growth factor receptor (EGFR) signaling pathway (p-value of overlap: 9.57×10⁻⁶). Specifically, R. delemar infection induced the expression of 18 genes that are known to be activated by EGFR signaling (FIG. 19B), providing evidence for the activation of EGFR protein in response to R. delemar infection.

Further support of the activation of EGFR signaling was provided by the predicted activation of mir-21 (p-value overlap: 8.6×10⁻¹⁻⁵, FIG. 19A), a microRNA that governs the expression of genes involved in many different biological processes (Buscaglia L E, Li Y. 2011. Apoptosis and the target genes of microRNA-21. Chin J Cancer 30:371-80.; Feng Y H, Tsao C J. 2016. Emerging role of microRNA-21 in cancer. Biomed Rep 5:395-402.; Li Y, Zhang J, Lei Y, Lyu L, Zuo R, Chen T. 2017. MicroRNA-21 in Skin Fibrosis: Potential for Diagnosis and Treatment. Mol Diagn Ther 21:633-642; Sekar D, Shilpa B R, Das A J. 2017. Relevance of microRNA 21 in Different Types of Hypertension. Curr Hypertens Rep 19:57; Sekar D, Venugopal B, Sekar P, Ramalingam K. 2016. Role of microRNA 21 in diabetes and associated/related diseases. Gene 582:14-8; Wang S, Wan X, Ruan Q. 2016. The MicroRNA-21 in Autoimmune Diseases. Int J Mol Sci 17). EGFR activation enhances the expression of mir-21 in lung epithelial cells (Seike M, Goto A, Okano T, Bowman E D, Schetter A J, Horikawa I, Mathe E A, Jen J, Yang P, Sugimura H, Gemma A, Kudoh S, Croce C M, Harris C C. 2009. MiR-21 is an EGFR-regulated anti-apoptotic factor in lung cancer in never-smokers. Proc Natl Acad Sci USA 106:12085-90). Our sequencing approach, which is geared toward the detection of long transcripts, does not allow the examination of microRNAs. However, in our infection model, 28 known repression targets and 5 known activation targets of mir-21 were down-regulated or up-regulated, respectively, 14 hours after inoculation relative to the uninfected control group. This down-regulation of mir-21 repressed genes is consistent with an EGFR stimulated increase in mir-21 expression. In the database used to perform the Upstream Regulator Analysis, none of the 33 mir-21 targets genes are annotated as EGFR targets and are therefore not included in FIG. 19C, so the total number of differentially expressed genes that provide evidence of EGFR pathway activation is 51.

EGFR signaling is activated during in vitro infection of airway epithelial cells. We have previously examined the transcriptional response of A549 human alveolar epithelial cells to infection with R. delemar at 6 and 16 h (Chibucos M C, Soliman S, Gebremariam T, Lee H, Daugherty S, Orvis J, Shetty A C, Crabtree J, Hazen T H, Etienne K A, Kumari P, O'Connor T D, Rasko D A, Filler S G, Fraser C M, Lockhart S R, Skory C D, Ibrahim A S, Bruno V M. 2016. An integrated genomic and transcriptomic survey of mucormycosis-causing fungi. Nat Commun 7:12218). Upstream regulator analysis of this in vitro RNA-seq dataset also revealed a significant overlap between genes that are differentially expressed following R. delemar infection and the known transcriptional targets of the EGFR signaling pathway (p-value: 4.33×10⁻² and 1.43×10⁻³ for 6 and 16 h respectively). Specifically, R. delemar infection induced changes in gene expression of 34 known downstream targets of EGFR signaling in a direction that is consistent with the activation of the EGFR (29 activated and 5 repressed; FIG. 19C).

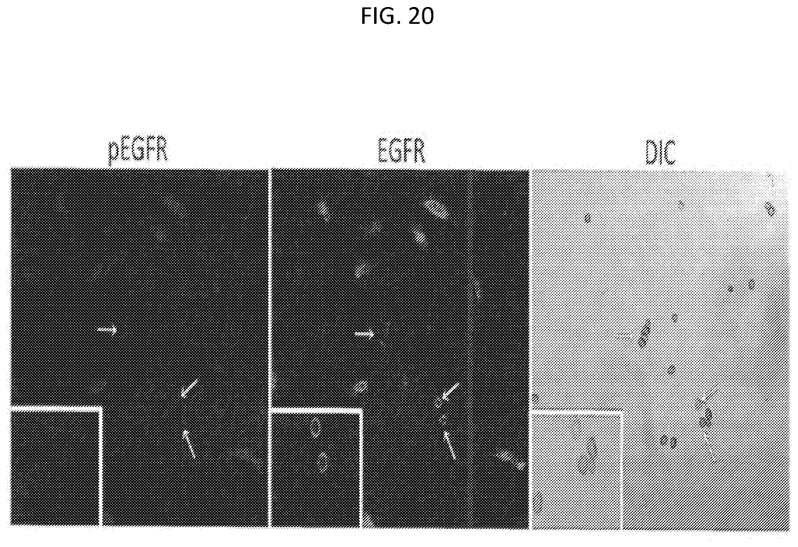

When EGFR is activated by ligand-binding, tyrosine residues at the intracellular carboxy-terminus become phosphorylated and the signal is transmitted to a variety of downstream signaling pathways (Decker Si, 1993. Transmembrane signaling by epidermal growth factor receptors lacking autophosphorylation sites. J Biol Chem 268:9176-9). To confirm the EGFR activation by an orthogonal approach, we used confocal microscopy to determine the localization of EGFR in R. delemar infected cells. We observed that both EGFR and phospho-EFGR, the phoshorylated (activated) form, co-localized with R. delemar spores during in vitro infection of A549 cells (FIG. 20). We did not observe co-localization when R. delemar spores were examined in the absence of A549 cells and EGFR did not co-localize with plastic beads that had been endocytosed by A549 cells. Taken together, these results are consistent with a model in which R. delemar interacts with EGFR and activates signaling in airway epithelial cells.

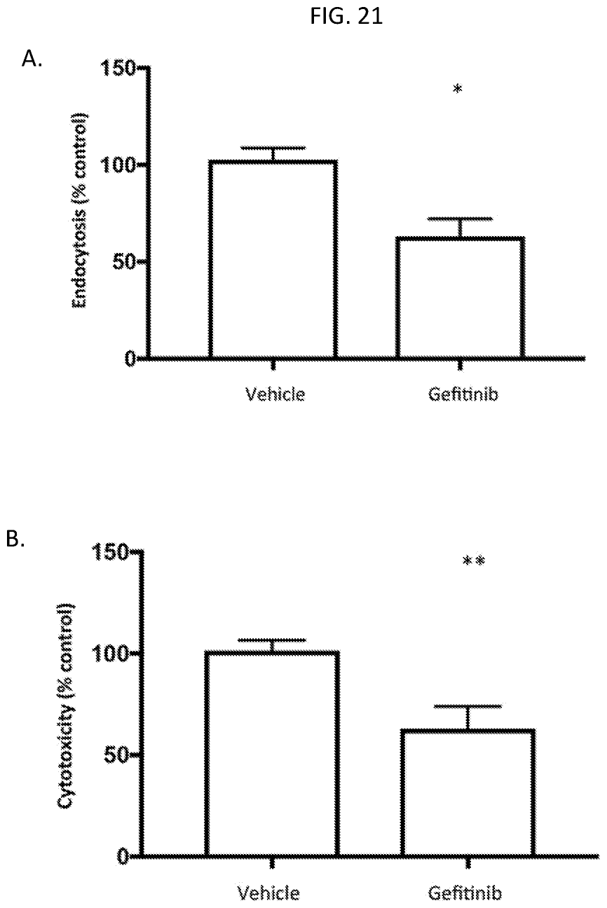

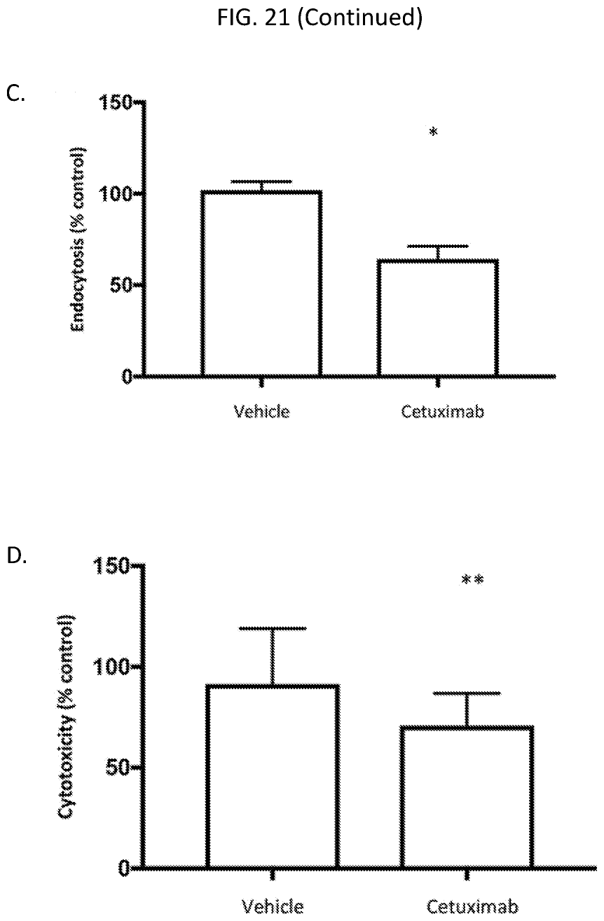

EGFR signaling governs the uptake of R. delemar and subsequent damage of airway epithelial cells. The predicted activation of EGFR and mir-21 signaling early during the infection process, the co-localization of activated EGFR with R. delemar as well as involvement of EGFR in the invasion of diverse microbial pathogens (Ho J, Moyes D L, Tavassoli M, Naglik J R. 2017. The Role of ErbB Receptors in Infection. Trends Microbiol 25:942-952), compelled us to explore the possibility that EGFR mediates the invasion of airway epithelial cells by R. delemar. Thus, we next tested whether blocking EGFR signaling would protect alveolar epithelial cells from invasion by R. delemar. We used Gefitinib, a clinically relevant EGFR kinase inhibitor, to study its effect on R. delemar-mediated endocytosis of alveolar epithelial cells and their subsequent damage. When A549 cells were pretreated for 1 h with 25 μM Gefitinib, endocytosis of R. delemar spores was significantly reduced compared to pre-treatment with vehicle alone (FIG. 21A). Pretreatment with Gefitinib also significantly reduced the R. delemar-induced damage of A549 cells, when assayed by LDH assay (FIG. 21B).

To complement our Gefitinib studies, which targets the intracellular tyrosine kinase domain of EGFR, we examined the ability of Cetuximab to block epithelial cell invasion. Cetuximab is a monoclonal antibody that recognizes the extracellular portion of EGFR and has been shown to block ligand-dependent activation of EGFR signalling (Vincenzi B, Zoccoli A, Pantano F, Venditti O, Galluzzo S. 2010. Cetuximab: from bench to bedside. Curr Cancer Drug Targets 10:80-95). When the same host cells were pretreated for 1 hour with 25 μg/ml Cetuximab, endocytosis of R. delemar spores and host cell damage were both significantly reduced compared to pre-treatment with an equivalent amount of IgG1 control antibody (FIG. 21C, D). Notably, Gefitinib or Cetuximab had no effect on R. delemar mycelial growth. Pre-treatment of R. delemar spore preparations with Gefitinib or Cetuximab prior to the infection did not reduce endocytosis or host cell damage. The concordance between the results of the endocytosis assay and the host cell damage assay is consistent with previous observations that endocytosis of R. delemar is a prerequisite for inducing host cell damage endothelial cells (Ibrahim A S, Spellberg B, Avanessian V, Fu Y, Edwards J E, Jr. 2005. Rhizopus oryzae adheres to, is phagocytosed by, and damages endothelial cells in vitro. Infect Immun 73:778-83). Collectively, these results indicate that EGFR signaling is required for maximal invasion of alveolar epithelial cells by R. delemar. However, our observations that Gefinitib or Cetuximab treatment did not completely block epithelial cell invasion suggests that other host receptor pathways also facilitate R. delemar invasion of airway epithelial cells.

EGFR signaling has also been shown to facilitate invasion of oral epithelial cells by Candida albicans which, like R. delemar, enters cells by induced endocytosis (Solis N V, Swidergall M, Bruno V M, Gaffen S L, Filler S G. 2017. The Aryl Hydrocarbon Receptor Governs Epithelial Cell Invasion during Oropharyngeal Candidiasis. MBio 8; Zhu W, Phan Q T, Boontheung P, Solis N V, Loo J A, Filler S G. 2012. EGFR and HER2 receptor kinase signaling mediate epithelial cell invasion by Candida albicans during oropharyngeal infection. Proceedings of the National Academy of Sciences of the United States of America 109:14194-9). The EGFR-dependent invasion of C. albicans requires the activation of the aryl hydrocarbon receptor (Ahr) and the subsequent activation of Src family kinases which in turn phosphorylate and activate EGFR ((Solis N V, Swidergall M, Bruno V M, Gaffen S L, Filler S G. 2017. The Aryl Hydrocarbon Receptor Governs Epithelial Cell Invasion during Oropharyngeal Candidiasis. MBio 8; Zhu W, Phan Q T, Boontheung P, Solis N V, Loo J A, Filler S G. 2012. EGFR and HER2 receptor kinase signaling mediate epithelial cell invasion by Candida albicans during oropharyngeal infection. Proceedings of the National Academy of Sciences of the United States of America 109:14194-9). To address whether the same mechanism is being employed to facilitate invasion of airway epithelial cells by R. delemar, we measured the effect of an Ahr inhibitor (CH-223191) and a Src inhibitor (Src Kinase Inhibitor I I) on endocytosis. Neither of the inhibitors altered the ability of A549 cells to endocytose R. delemar spores. These results suggest that the mechanism of activation of EGFR in epithelial cells is distinct from the activation of EGFR by C. albicans.

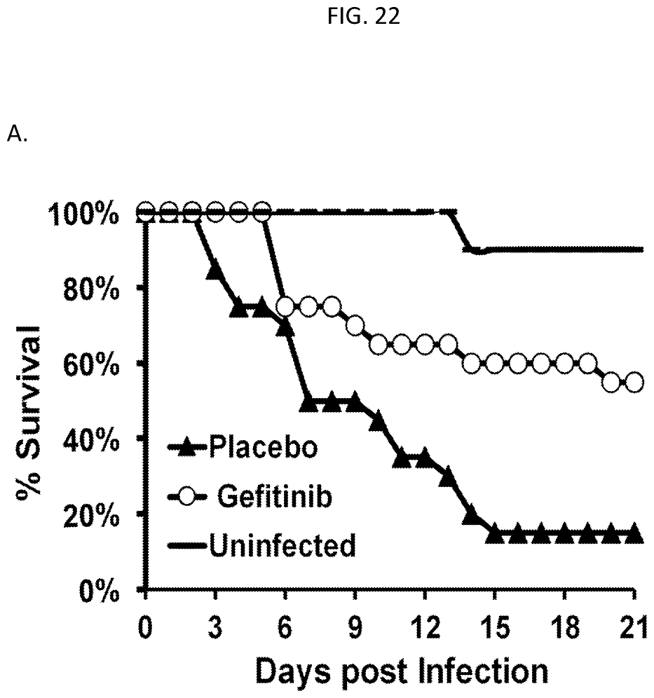

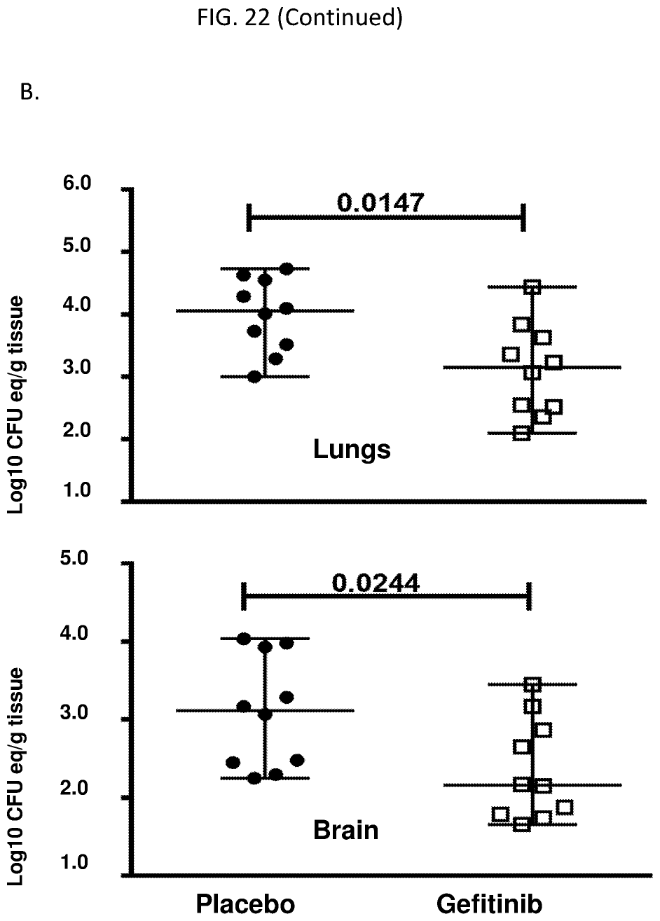

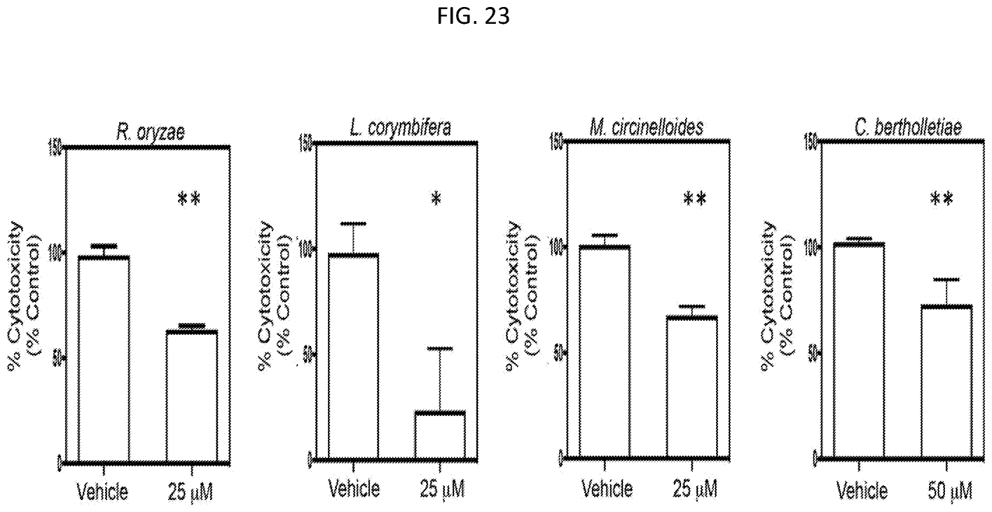

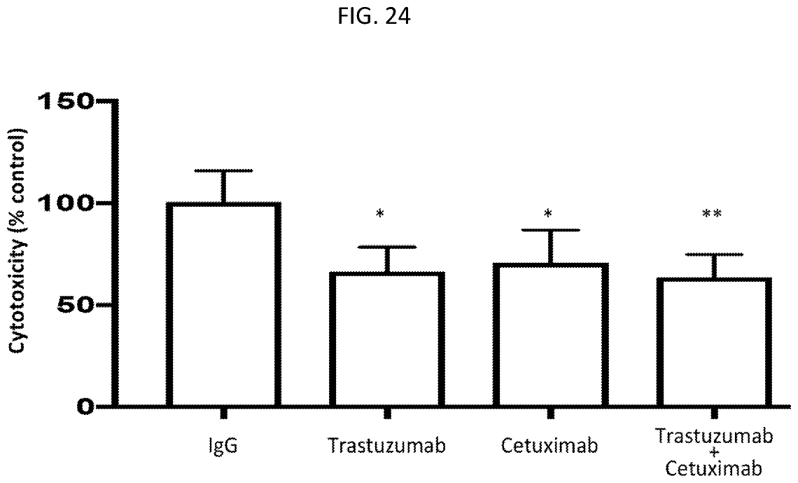

Gefitinib treatment increases survival of mice with mucormycosis. We next sought to determine if EGFR signaling governs the establishment and/or progression of mucormycosis in a well-established in vivo murine model of mucormycosis. Unfortunately, mice harboring deletions in EGFR die within the first eight days of life (Miettinen P J, Berger J E, Meneses J, Phung Y, Pedersen R A, Werb Z, Derynck R. 1995. Epithelial immaturity and multiorgan failure in mice lacking epidermal growth factor receptor. Nature 376:337-41) thus precluding our ability to test the receptor in a traditional mouse gene deletion experiment. Furthermore, there are no published lung-specific deletion models for EGFR. Therefore, we infected neutropenic mice intratracheally with R. delemar (Luo G, Gebremariam T, Lee H, French S W, Wiederhold N P, Patterson T F, Filler S G, Ibrahim A S. 2013. Efficacy of liposomal amphotericin B and posaconazole in intratracheal models of murine mucormycosis. Antimicrob Agents Chemother 57:3340-7) and treated with 10 mg/kg of Gefitinib (Lynch T J, Bell D W, Sordella R, Gurubhagavatula S, Okimoto R A, Brannigan B W, Harris P L, Haserlat S M, Supko J G, Haluska F G, Louis D N, Christiani D C, Settleman J, Haber D A. 2004. Activating mutations in the epidermal growth factor receptor underlying responsiveness of non-small-cell lung cancer to gefitinib. N Engl J Med 350:2129-39), or vehicle alone (placebo), for 5 consecutive days starting 4 h post infection. We chose to begin the intervention at 4 h because invasion of lung epithelial cells is an early event in the infection. Placebo-treated mice had a median survival time of 8 days and an 85% mortality as early as Day 15 post infection. In contrast, mice treated with Gefitinib had a median survival time of >21 days and 55% of the mice survived the infection by Day 21 when the experiment was terminated and the surviving mice appearing healthy (FIG. 22A). Corroborating the prolonged survival effect of Gefitinib, mice treated with EGFR inhibitor had ˜1 log reduction in their organs (i.e. lungs and brains) when compared to placebo-treated mice (FIG. 22B). These results validate our in vitro observations and support the potential of targeting EGFR signaling as a novel therapeutic strategy for mucormycosis by immediately repurposing currently FDA-approved cancer drugs.

### Materials and Methods

Fungal strains and host cells. Rhizopus arrhizus var. delemar (R. delemar) strain 99-880 was grown on PDA plates for 3-5 days at 37° C. Spores were collected in endotoxin-free Dulbecco PBS (DPBS), washed with endotoxin-free DPBS, and counted with a hemoccytometer to prepare the final inocula. To form germlings, spores were germinated in yeast-extract-peptone-dextrose (YPD) with shaking for 1 h at 37° C. Germlings were washed twice with endotoxin-free DPBS. The A549 type II pneumocyte cell line (cells were grown in tissue culture dishes in F12k medium with L-glutamine plus 10% fetal bovine serum (FBS).

Drugs. Src Kinase Inhibitor I I [CAS 459848-35-2 Calbiochem (Millipore; catalog #567806)]

Murine models of Mucormycosis. Male ICR mice (20-25 g from Envigo) were immunosuppresed by cyclophosphamide (200 mg/kg administered intraperotenually [i.p]) and cortisone acetate (500 mg/kg, adminstered subcutaneously) given on days −2, +3, and +9 relative to infection. This treatment resulted in 16 days of pancytopenia (Luo G, Gebremariam T, Lee H, French S W, Wiederhold N P, Patterson T F, Filler S G, Ibrahim A S. 2013. Efficacy of liposomal amphotericin B and posaconazole in intratracheal models of murine mucormycosis. Antimicrobial agents and chemotherapy 57:3340-7). To control for bacterial infection, immunosuppressed mice received 50 mg/L Baytrial (enrofloxacin: Bayer, Leverkusen, Germany) ad libitum on Day −3 thorough Day 0, after which Baytril was replaced with daily ceftazidime (5 mg/mouse) treatment adminstered subcutaneously through day+13 days post infection. Mice were infected with 2.5×10⁵ spores of R. delemar 99-880 in 25 μL PBS given intratracheally as previously described (Luo G, Gebremariam T, Lee H, French S W, Wiederhold N P, Patterson T F, Filler S G, Ibrahim A S. 2013. Efficacy of liposomal amphotericin B and posaconazole in intratracheal models of murine mucormycosis. Antimicrobial agents and chemotherapy 57:3340-7). Treatment with Gefitinib (10 mg/kg dissolved in dimethylacetamide [DMA]:PEG300 [10%:90%] and adminstered via i.p. route) started 4 hours post infection and continued once daily through Day +4. Placebo mice received DMA:PEG300. Survival of mice served as the primary endpoint with moribund mice humanely euthanized. To determine effect of treatment on tissue fungal burden mice were immunosuppressed and infected as above. Gefitinib treatment started 4 post infection and continued through Day +3. On Day +4, mice were sacrificed and lungs and brains, representing primary and secondary target organs (Luo G, Gebremariam T, Lee H, French S W, Wiederhold N P, Patterson T F, Filler S G, Ibrahim A S. 2013. Efficacy of liposomal amphotericin B and posaconazole in intratracheal models of murine mucormycosis. Antimicrobial agents and chemotherapy 57:3340-7), were collected and processed for tissue fungal burden by qPCR (Ibrahim A S, Bowman J C, Avanessian V, Brown K, Spellberg B, Edwards J E, Jr., Douglas C M. 2005. Caspofungin inhibits Rhizopus oryzae 1,3-beta-D-glucan synthase, lowers burden in brain measured by quantitative PCR, and improves survival at a low but not a high dose during murine disseminated zygomycosis. Antimicrobial agents and chemotherapy 49:721-7.) Values are expressed as Log₁₀ spore equivalents per gram tissue.

All animal studies were approved by the Institutional Animal Care and Use Committee (IACUC) of the Los Angeles Biomedical Research Institute at Harbor-UCLA Medical Center according to the NIH guidelines for animal housing and care. Approval reference number 21125.

Isolation of RNA from lung tissue. Male ICR mice were immunosuppresed and infected as above. Lungs were harvested 14 or 24 h post infection and flash frozen in liquid nitrogen prior to extracting total RNA using Tri Reagent solution (Ambion).

RNA-seq and gene expression analysis. Sequencing libraries (non-strand-specific, paired end) were prepared with the TruSeq RNA sample prep kit (Illumina). The total RNA samples were subjected to poly(A) enrichment as part of the TruSeq protocol. 150 nucleotides of sequence were determined from both ends of each cDNA fragment using the HiSeq platform (Illumina) per the manufacturer's protocol. Sequencing reads were annotated and aligned to the UCSC mouse reference genome (mm10, GRCm38.75) using TopHat2 (Kim D, Pertea G, Trapnell C, Pimentel H, Kelley R, Salzberg S L. 2013. TopHat2: accurate alignment of transcriptomes in the presence of insertions, deletions and gene fusions. Genome Biol 14:R36). The alignment files from TopHat2 were used to generate read counts for each gene, and a statistical analysis of differential gene expression was performed using the EdgeR package from Bioconductor (Robinson M D, McCarthy D J, Smyth G K. 2010. edgeR: a Bioconductor package for differential expression analysis of digital gene expression data. Bioinformatics 26:139-40). A gene was considered differentially expressed if the P value for differential expression was less than 0.05. To identify modulated signal transduction pathways, we used the upstream regulator analytic of IPA (Ingenuity Systems) to identify signaling proteins that are potentially activated or repressed during the course of infection. This analysis determines the overlap between lists of differentially expressed genes and an extensively curated database of regulator-target gene relationships. It then considers the direction of the gene expression changes to make predictions about activation or repression of specific pathways.

Measurement of R. delemar-induced host cell damage. R. delemar-induced A549 cell damage was quantified using the Pierce LDH Assay with slight modifications to the manufacturer's protocol. Briefly, A549 cells were grown in 96-well tissue culture plates for 18-24 hours. Cells were then pre-treated for 1 hour with Gefitinib (25 μM) or Cetuximab (25 μg/ml), and infected with 2×10⁶ germlings suspended in 150 μl F12K+10% FBS. For controls, host cells will be incubated with DMSO (the solvent used to reconstitute the inhibitor) or 25 μg/ml of mouse IgG antibody in parallel. After 24 hours of incubation at 37° C., 50 μl of the cell culture supernatant was collected from uninfected, infected, and fungi-only control wells and transferred to a 96 well plate to assay for LDH activity. Lysis buffer was added to all infected wells and incubated for 45 min at 37° C. After lysis, 50 μl of cell culture supernatant was transferred to a 96 well plate and used for the LDH Assay Kit per protocol. LDH release was calculated as follows: % Cytotoxicity=[Experimental release−Fungal Cells Spontaneous Control−Host Cells Spontaneous Control]/[Host Cell Maximum Control−Fungal Cells Maximum Control−Host Cells Spontaneous Control]×100. LDH is a cytosolic enzyme but will be released into the cell culture medium upon cell membrane damage. The amount of extracellular LDH is proportional to the amount of cell damage.

Measurement of host cell endocytosis. 12-mm glass coverslips were seeded with A549 alveolar epithelial cells. Cells were then pre-treated for 1 h with Gefitinib (25 μM) or Cetuximab (25 μg/ml). For controls, host cells will be incubated with DMSO (the solvent used to reconstitute the inhibitor) or 25 μg/ml mouse IgG antibody in parallel. Host cells were then infected with 2×10⁵ R. delemar spores. After incubation for 3 h, cells were fixed in 3% paraformaldehyde and stained for 1 h with 1% Uvitex, which specifically binds to chitin in the fungal cell wall. After washing with PBS, coverslips were mounted on a glass slide with a drop of ProLong Gold anti-fade reagent (Molecular Probes) and sealed. The total number of cell-associated organisms (i.e. fungi adhering to the monolayer) per high-powered field were determined by phase-contrast microscopy. The same field will be examined by epifluorescence microscopy, and the number of brightly fluorescent, uninternalized fungi were determined. The number of endocytosed organisms was calculated by subtracting the number of fluorescent fungi from the total number of visible fungi. At least 400 organisms were counted per treatment group in at least 15 different fields per coverslip. Experiments were performed in duplicate or triplicate on at least two separate days.

Confocal Microscopy. The accumulation of epithelial cell EGFR and pEGFR around R. delemar was visualized using the Zeiss LSM Duo Confocal Microscopy system. 12 mm glass coverslips in 12 well dishes were seeded with A549 alveolar epithelial cells and infected with 2×105 R. delemar. After incubation at 37° C., cells were washed with HBSS and fixed with 3% paraformaldehyde. Cells were blocked and incubated with 1:500 mouse anti-EGFR (Santa Cruz sc-373746) and 1:500 rabbit anti-pEGFR (Cell Signaling 3777). Coverslips were washed and counterstained with 1:500 Alexa Fluor 546—labeled goat anti-mouse IgG and Alexa Fluor 488—labeled goat anti-rabbit IgG. After washing, coverslips were mounted on a glass slide with ProLong Gold antipode reagent (Molecular Probes) and viewed by z-stacking using the Zeiss LSM Duo Confocal Microscopy system.

Statistical Analyses. In vitro experiments were performed in triplicate on two or three separate days. Data were expressed as median±interquartile range. Treatment groups were compared to controls using the Wilcoxon rank-sum test and P values <0.05 were considered significant. For the murine studies, survival of mice was analyzed using the Log Rank test, whereas differences in tissue fungal burden were analyzed by the Wilcoxon Rank Sum test using Graph pad Prism 6. P values of <0.05 were considered significant.

### Example 2. Growth Factor Receptor Targeting to Treat Mucormycosis

Using a transcriptome-guided approach to understand the Mucorales-host interaction, we observed the upregulation of signaling pathways that originate with different growth factor receptor (GFRs); specifically, PDGFR, EGFR and ErbB2. Each of these pathways is a well-established target of various FDA-approved drugs that are currently used to treat various forms of cancer. Here, we will explore the therapeutic potential of targeting GFR pathways by repurposing these existing, often-used, drugs to treat mucormycosis. Experiments will also focus on understanding the molecular mechanism behind the pathway activations with the goal of optimizing treatment regimens through combinatorial approaches.

1: To investigate the therapeutic potential of targeting GFRs to treat mucormycosis and to identify the underlying mechanisms of pathway activation.

Rationale and Approach: Our preliminary data show that inhibiting the receptor tyrosine kinase activity of EGFR with gefitinib (an FDA-approved drug) increases survival in a neutropenic mouse model of mucormycosis and blocking the activity of ErbB2 and PDGFR also inhibits infection in vitro. In this aim, we will comprehensively assess the requirement of each of these growth factor receptors on fungal invasion in vivo and determine the fungal component that is triggering the signaling. Understanding the fungal secreted or cell wall molecules that trigger GFR activation will provide important leads to optimize the efficacy of the GFR inhibitors.

2: To identify fungal targets and determine their therapeutic potential to treat mucormycosis.

Rationale and Approach: Which fungal genes are expressed during infection, at which stage, and in which host environments? What do they do? These are fundamental questions for all microbial pathogens that have gone largely unanswered because of low abundance of pathogen RNA that can be extracted from infected tissues. Our in vitro transcriptome of Mucorales interacting with alveolar epithelial cells and preliminary in vivo experiments implicate several fungal genes in mucormycosis pathogenesis, including endo-1,3β-glucosidase and a BCR-1 homolog. We will confirm these results by profiling fungal gene expression in vivo. Using our neutropenic mouse model of mucormycosis, we will selectively enrich Mucorales mRNA away from host mRNA from infected tissue and perform RNA-seq analysis to measure temporal gene expression patterns in the lungs. This in vivo transcriptomic data will be combined with comparative genomics to identify a set of fungal genes that are likely to be important for the host pathogen interaction. We will assess the role of each gene by using RNAi and/or CRISPR-Cas9 to generate strains with attenuated expression of each gene and testing virulence during in vitro infection and in our neutropenic murine model of mucormycosis. The result of this aim will be a functionally validated list of desperately needed therapeutic targets to treat this devastating disease.

PREMISE AND IMPACT: Mucormycosis has a remarkably high morbidity/mortality and is on the rise, representing the third most common fungal infection in hematologic malignancy patients. Our studies confirm the importance of GFRs in the pathogenesis of mucormycosis and FDA-approved drugs targeting GFRs are likely to play an immediate role in improving care for mucormycosis patients. The proposed studies also adopt a holistic approach to identify genes/pathways that promote the disease in clinically relevant animal models. Thus, the generated data will direct the development of new therapies targeting genes or gene products that are essential for the virulence of Mucorales.

Research Strategy

A. Significance

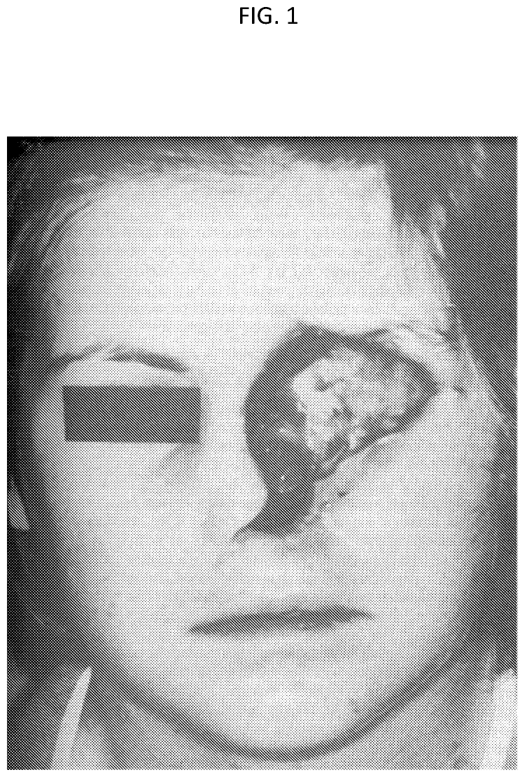

Mucormycosis is an increasingly common, highly lethal fungal infection with limited treatments. Despite mandatory disfiguring surgical debridement, such as excision of the eye in patients with rhinocerebral mucormycosis (FIG. 1), and adjunctive antifungal therapy, the overall mortality of mucormycosis remains ≥50%. It approaches 100% in patients with disseminated disease, or persistent neutropenia (Spellberg, B., Edwards Jr., J. & Ibrahim, A. Novel perspectives on mucormycosis: pathophysiology, presentation, and management. Clin Microbiol Rev 18, 556-569 (2005); Gleissner, B., Schilling, A., Anagnostopolous, I., Siehl, I. & Thiel, E. Improved outcome of zygomycosis in patients with hematological diseases? Leuk Lymphoma 45, 1351-1360 (2004); Kauffman, C. A. Zygomycosis: reemergence of an old pathogen. Clin Infect Dis 39, 588-590, doi:10.1086/422729 (2004); Kontoyiannis, D. P., Wessel, V. C., Bodey, G. P. & Rolston, K. V. Zygomycosis in the 1990s in a tertiary-care cancer center. Clin Infect Dis 30, 851-856 (2000); Marr, K. A., Carter, R. A., Crippa, F., Wald, A. & Corey, L. Epidemiology and outcome of mold infections in hematopoietic stem cell transplant recipients. Clin Infect Dis 34, 909-917 (2002); Siwek, G. T. et al. Invasive zygomycosis in hematopoietic stem cell transplant recipients receiving voriconazole prophylaxis. Clin Infect Dis 39, 584-587 (2004)).

There has been an alarming rise in the incidence of mucormycosis at major US transplant centers. The number of cases over a 15-year period has more than doubled (Gleissner, B., Schilling, A., Anagnostopolous, I., Siehl, I. & Thiel, E. Improved outcome of zygomycosis in patients with hematological diseases? Leuk Lymphoma 45, 1351-1360 (2004); Marr, K. A., Carter, R. A., Crippa, F., Wald, A. & Corey, L. Epidemiology and outcome of mould infections in hematopoietic stem cell transplant recipients. Clin Infect Dis 34, 909-917 (2002); Kontoyiannis, D. P., Wessel, V. C., Bodey, G. P. & Rolston, K. V. Zygomycosis in the 1990s in a tertiary-care cancer center. Clin Infect Dis 30, 851-856 (2000)). Prevalence rates are up to 8% in autopsied patients with leukemia (Greenberg, R. N., Scott, L. J., Vaughn, H. H. & Ribes, J. A. Zygomycosis (mucormycosis). emerging clinical importance and new treatments. Curr Opin infect Dis 17. 517-525, doi:00001432-200412000-00003 [pii] (2004)). A French study showed a 70% increase in mucormycosis cases between 1997 and 2006 (Bitar, D. et al. Increasing incidence of zygomycosis (mucormycosis), France, 1997-2006. Emerg Infect Dis 15, 1395-1401, doi:10.3201/eid1509.090334 (2009)). Further, data from a hospital in India showed ≥400% increase in mucormycosis incidence between 1991 and 2007, mainly among DKA patients (Chakrabarti, A., Chatterjee, S. S. & Shivaprakash, M. R. Overview of opportunistic fungal infections in India. Nippon Ishinkin Gakkai Zasshi 49, 165-172, doi:JST.JSTAGE/jjmm/49.165 [pii] (2008); Chakrabarti, A. et al. Invasive zygomycosis in India: experience in a tertiary care hospital. Postgrad Med J 85, 573-581, doi:10.1136/pgmj.2008.076463 (2009)). Strikingly, a study reviewing cases in India for the past five decades predicted a prevalence of 0.14 cases/1000 population (˜200,000 cases/year) (Chakrabarti, A. & Singh, R. Mucormycosis in India: unique features. Mycoses 57 Suppl 3, 85-90, doi:10.1111/myc.12243 (2014)). Outbreaks of mucormycosis are associated with natural disasters (e.g. Indian ocean tsunami and the Joplin tornado) (Neblett Fanfair, R. et al. Necrotizing cutaneous mucormycosis after a tornado in Joplin, Missouri, in 2011. N Engl J Med 367, 2214-2225, doi:10.1056/NEJMoa1204781; Andresen, D. et al. Multifocal cutaneous mucormycosis complicating polymicrobial wound infections in a tsunami survivor from Sri Lanka. Lancet 365, 876-878, doi:10.1016/S0140-6736(05)71046-1 (2005).) and in soldiers injured in combat operations (Tully, C. C., Romanelli, A. M., Sutton, D. A., Wickes, B. L. & Hospenthal, D. R. Fatal Actinomucor elegans var. kuwaitiensis infection following combat trauma. J Clin Microbiol 47, 3394-3399, doi:JCM.00797-09 [pii] 10.1128/JCM.00797-09 (2009); Warkentien, T. et al. Invasive mold infections following combat-related injuries. Clin Infect Dis 55, 1441-1449, doi:cis749 [pii]). Mucormycosis cases are also highly underestimated because of the lack of a reliable diagnostic assay and the absence of federal requirements for reporting fungal infections (Walsh, T. J. et al. Development of new strategies for early diagnosis of mucormycosis from bench to bedside. Mycoses 57, 2-7, doi:10.1111/myc.12249 (2014)).

The five most common forms of mucormycosis, based on anatomical site, are rhino-cerebral, pulmonary, cutaneous, gastrointestinal and disseminated. Pulmonary mucormycosis occurs most often in neutropenic patients and it has a mortality rate of 76% (Roden, M. M. et al. Epidemiology and outcome of zygomycosis: a review of 929 reported cases. Clin Infect Dis 41, 634-653 (2005)). In the clinic, the features of pulmonary mucormycosis are virtually indistinguishable from pulmonary aspergillosis. However, Mucorales are resistant to voriconazole, a drug that is used to treated pulmonary aspergillosis (Capilla, J., Serena, C., Pastor, F. J., Ortoneda, M. & Guarro, J. Efficacy of voriconazole in treatment of systemic scedosporiosis in neutropenic mice. Antimicrob Agents Chemother 47, 3976-3978 (2003); Chamilos, G., Marom, E. M., Lewis, R. E., Lionakis, M. S. & Kontoyiannis, D. P. Predictors of pulmonary zygomycosis versus invasive pulmonary aspergillosis in patients with cancer. Clin Infect Dis 41, 60-66, doi:C1D35730 [pii] 10.1086/430710 (2005)).

Antifungal treatments for mucormycosis represent a significant unmet clinical need. Currently, there are only two antifungal agents approved by the USA FDA for first-line therapy for treating mucormycosis: AmB and isavuconazole. AmB has serious side effects, such as nephrotoxicity, and very limited clinical success. Although isavuconazole is associated with significantly less side effects, it is not superior to AmB-treatment. The unacceptably high mortality rate of 50-100%, limited options for therapy, and the extreme morbidity of highly disfiguring surgical therapy, make it imperative to increase our understanding of these pathogen, to enable the development of alternative strategies to treat and prevent mucormycosis.

B. Innovation

We will define unique and previously unexplored pathogenic mechanisms that govern the interaction between fungal and alveolar epithelial cells during the initiation of mucormycosis. By testing the hypothesis that molds hijack host GFRs signaling pathways to facilitate tissue invasion, we are exploring the potential of repurposing FDA-approved cancer treatments to treat mucormycosis.

In addition to this hypothesis-driven research in Aim 1, discovery-driven research is badly needed. Given the paucity of information about mucormycosis, we desperately need discovery-driven research. We can start the 26.2 mile marathon at the 0 mile mark, we can start at the 13 mile marker by leveraging research done on Candida and Saccharomyces over the last decades, or we can start at mile 26 using the latest genomic techniques to discover new and unique biology of these organisms. Thus, we propose to use state-of-the-art techniques to assess the in vivo transcriptional profile of Mucorales genes that are expressed during infection in clinically relevant animal models (Luo, G. et al. Efficacy of liposomal amphotericin B and posaconazole in intratracheal models of murine mucormycosis. Antimicrob Agents Chemother 57, 3340-3347, doi:10.1128/AAC.00313-13 (2013)), providing new insight into the pathogenesis of mucormycosis. This wealth of information will identify novel potential drug and immunotherapeutic targets as well as potential biomarkers to exploit in developing rapid diagnosis of a disease that is poorly diagnosed.

C. Approach

1: To investigate the therapeutic potential of targeting GFRs to treat mucormycosis and to identify the underlying mechanisms of pathway activation.

Background and Rationale. We need to determine the molecular mechanisms by which Mucorales spp. interact with host receptors on alveolar epithelial cells in order to understand how these fungi invade the host tissues to initiate pulmonary infection.

Rhizopus invasion and damage of epithelial cells are essential for initiating pulmonary mucormycosis. Commonly, mucormycosis is acquired via inhalation, which results in either rhino-orbital disease or pulmonary mucormycosis (Spellberg, B., Edwards Jr., J. & Ibrahim, A. Novel perspectives on mucormycosis: pathophysiology, presentation, and management. Clin Microbiol Rev 18, 556-569 (2005); Roden, M. M. et al. Epidemiology and outcome of zygomycosis: a review of 929 reported cases. Clin Infect Dis 41, 634-653 (2005); Kwon-Chung, K. J. & Bennett, J. E. in Medical Mycology 524-559 (Lea & Febiger, 1992)). In the absence of functional phagocytes that efficiently eliminate fungal spores and hyphae (Diamond, R. D. & Haudenschild, C. C. Monocyte-mediated serum-independent damage to hyphal and pseudohyphal forms of Candida albicans in vitro. J Clin Invest 67, 173-182. (1981); Waldorf, A. R. Pulmonary defense mechanisms against opportunistic fungal pathogens. Immunology Series 47, 243-271 (1989); Waldorf, A. R., Ruderman, N. & Diamond, R. D. Specific susceptibility to mucormycosis in murine diabetes and bronchoalveolar macrophage defense against Rhizopus. Journal of Clinical Investigation 74, 150-160 (1984)), the infection can rapidly progress into a hematogenous disseminated disease. Because of the propensity of Mucorales to invade blood vessels, thrombosis and tissue necrosis commonly occurs (Spellberg, B., Edwards Jr., J. & Ibrahim, A. Novel perspectives on mucormycosis: pathophysiology, presentation, and management. Clin Microbiol Rev 18, 556-569 (2005); Kwon-Chung, K. J. & Bennett, J. E. in Medical Mycology 524-559 (Lea & Febiger, 1992); Ibrahim, A. S., Edwards, J. E. J. & Filler, S. G. in Clinical mycology (eds W. E Dismukes, P. G. Pappas, & J. D. Sobel) 241-251 (Oxford University Press, 2003)). Hence, penetration through, and damage of airway epithelial cells to initiate the infection, is a critical step in the organism's pathogenicity.

The Ibrahim laboratory has demonstrated that Mucorales, including Rhizopus spp., which are the most common causative agent of mucormycosis, invade human umbilical vein endothelial cells (HUVEC) by binding to the host GRP78 protein (Liu, M. et al. The endothelial cell receptor GRP78 is required for mucormycosis pathogenesis in diabetic mice. J Clin Invest 120, 1914-1924, doi:42164 [pii]10.1172/JCI42164). Previously known to be a cellular chaperone, GRP78 is also expressed on the cell surface (Liu, M. et al. The endothelial cell receptor GRP78 is required for mucormycosis pathogenesis in diabetic mice. J Clin Invest 120, 1914-1924, doi:42164 [pii]10.1172/JCI42164; Wang, M., Wey, S., Zhang, Y., Ye, R. & Lee, A. S. Role of the unfolded protein response regulator GRP78/BiP in development, cancer, and neurological disorders. Antioxid Redox Signal 11, 2307-2316, doi:10.1089/ARS.2009.2485 (2009)) where it functions as a receptor for several infectious agents (Liu, M. et al. The endothelial cell receptor GRP78 is required for mucormycosis pathogenesis in diabetic mice. J Clin Invest 120, 1914-1924, doi:42164 [pii]10.1172/JCI42164; Jindadamrongwech, S., Thepparit, C. & Smith, D. R. Identification of GRP 78 (BiP) as a liver cell expressed receptor element for dengue virus serotype 2. Arch Virol 149, 915-927, doi:10.1007/s00705-003-0263-x (2004); Triantafilou, K., Fradelizi, D., Wilson, K. & Triantafilou, M. GRP78, a coreceptor for coxsackievirus A9, interacts with major histocompatibility complex class I molecules which mediate virus internalization. J Virol 76, 633-643 (2002)). We also recently made the seminal discovery that the fungal ligand that binds to GRP78 is CotH3, a cell surface protein expressed on spores and hyphae (Gebremariam, T. et al. CotH3 mediates fungal invasion of host cells during mucormycosis. J Clin Invest 124, 237-250, doi:71349 [pii] 10.1172/JCI71349).

The importance of the GRP78/CotH3 interactions is emphasized by the observation that polyclonal antibodies against GRP78 or CotH3 partially protect mice from mucormycosis (Liu, M. et al. The endothelial cell receptor GRP78 is required for mucormycosis pathogenesis in diabetic mice. J Clin Invest 120, 1914-1924, doi:42164 [pii]10.1172/JCI42164; Gebremariam, T. et al. CotH3 mediates fungal invasion of host cells during mucormycosis. J Clin Invest 124, 237-250, doi:71349 [pii] 10.1172/JCI71349). These studies demonstrate the feasibility of using antibody-based therapy against mucormycosis to ameliorate the disease in mice. However, the partial protection afforded by these antibodies also emphasizes the need to enhance this passive immunization strategy. While antibodies targeting endothelial cell GRP78-CotH3 interactions are likely to impede the progression of the disease, therapeutic strategies targeting initiation of the infection (e.g. epithelial cell interaction), are highly likely to be additive or synergistic with CotH3 antibodies.

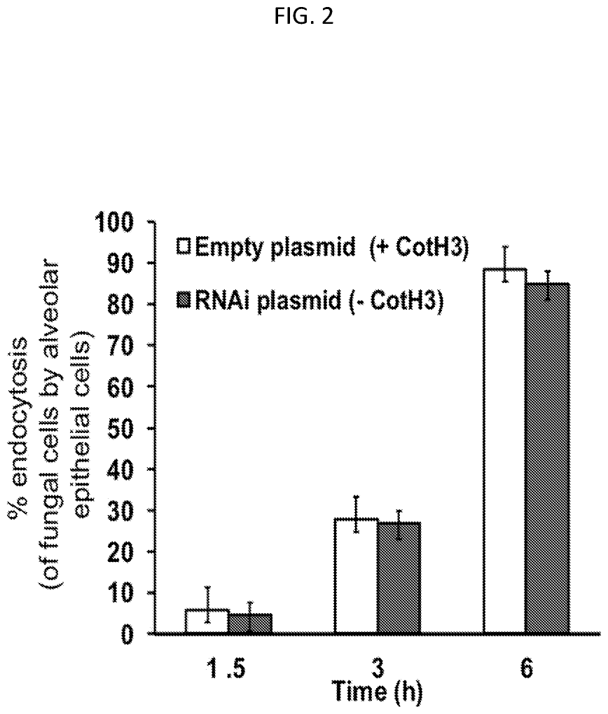

Our interaction studies of Rhizopus delemar 99-880 (formerly classified as Rhizopus oryzae) (Abe, A., Oda, Y., Asano, K. & Sone, T. Rhizopus delemar is the proper name for Rhizopus oryzae fumaric-malic acid producers. Mycologia 99, 714-722 (2007)) with alveolar epithelial cells (A549) revealed that the fungus is able to invade the cells as early as 90 minutes post incubation with almost complete invasion of the host monolayer within 6 h (FIG. 2). More importantly, a R. delemar with attenuated CotH2/3 expression (Gebremariam, T. et al. CotH3 mediates fungal invasion of host cells during mucormycosis. J Clin Invest 124, 237-250, doi:10.1172/JCI71349 (2014)), demonstrated similar ability in invading alveolar epithelial cells to R. delemar wild-type strain (FIG. 2). These studies suggested that R. delemar invade alveolar epithelial cells by a mechanism that is likely to involve the interaction of different host receptors/fungal ligands than that employed by the fungus during homogenous dissemination.

We conducted whole genome transcriptomics on Rhizopus or Mucor species (together these two genera are responsible for >90% of mucormycosis cases) (Ribes, J. A., Vanover-Sams, C. L. & Baker, D. J. Zygomycetes in human disease. Clin Microbiol Rev 13, 236-301 (2000)) while interacting with the alveolar epithelial cells. We found three host growth factor receptors (GFR) pathways; PDGFR, EGFR and ErbB2, to be highly induced by Mucorales (Chibucos, M. C. et al. An integrated genomic and transcriptomic survey of mucormycosis-causing fungi. Nature communications 7, 12218, doi:10.1038/ncomms12218 (2016)). Our subsequent confirmatory studies (Preliminary data) indicate that these GRFs are likely hijacked by Mucorales during invasion of alveolar epithelial cells. We plan to determine the role of each of these GFRs in the invasion of lung epithelial cells by Mucorales. Our long-range goal is to use this knowledge to develop new therapeutic strategies against this lethal infection. Notably, for each of these growth factor receptors, there exists a targeted, FDA-approved therapy that is currently being used in the clinic which means that they have already been scrutinized for safety. Our preliminary findings raise the exciting possibility that these currently available drugs can be repurposed to treat mucormycosis.

### Results

Mucorales invade airway epithelial cells and vascular endothelial cells via different mechanisms.

We previously found that R. delemar-mediated HUVEC injury is reliant on host cell GRP78 and fungal CotH3 interactions during invasion of the endothelium (Liu, M. et al. The endothelial cell receptor GRP78 is required for mucormycosis pathogenesis in diabetic mice. J Clin Invest 120, 1914-1924, doi:42164 [pii]10.1172/JCI42164; Gebremariam, T. et al. CotH3 mediates fungal invasion of host cells during mucormycosis. J Clin Invest 124, 237-250, doi:71349 [pii] 10.1172/JCI71349). In contrast, R. delemar invades A549 alveolar epithelial cells within 6 h of incubation in a process that is independent of the fungal CotH3 since a CotH3-reduced expression mutant was as invasive as R. delemar harboring the empty plasmid to A549 cells (FIG. 2). Thus, the CotH3/GRP78 interaction is not required for invasion of alveolar epithelial cells.

In vitro host transcriptomics upon inoculation. To begin to understand the early mechanisms of alveolar epithelial cell infection (i.e. invasion and subsequent damage) we performed RNA-seq on Mucorales (R. delemar, R. oryzae, or, Mucor circinelloides) infected monolayers of A549 cells at early and late times post infection.

Our analysis of host gene expression focused on modulation of regulatory networks by performing an Upstream Regulator Analysis to identify signal transduction pathways that might be modulated during infection relative to uninfected controls. We have successfully used this approach previously to gain insights into the pathogenesis of candidiasis (Bruno, V. M. et al. Transcriptomic analysis of vulvovaginal candidiasis identifies a role for the NLRP3 inflammasome. MBio 6, doi:10.1128/mBio.00182-15 (2015); Liu, Y. et al. New signaling pathways govern the host response to C. albicans infection in various niches. Genome Research 25, 679-689, doi:10.1101/gr.187427.114 (2015)). Our upstream regulator analysis of host gene expression indicated the activation of several pathways involved in inflammation and stress signaling such as TNF and NUPR1. Notably, all 3 Mucorales strains activated known host GFRs signaling pathways: PDGF BB (platelet-derived growth factor BB homodimer), ERBB2 (erb-b2 tyrosine kinase 2; also known as HER2), (data not shown; published by our groups, Nature Communications, 2016) (Chibucos, M. C. et al. An integrated genomic and transcriptomic survey of mucormycosis-causing fungi. Nature communications 7, 12218, doi:10.1038/ncomms12218 (2016)).

PDGFs are serum proteins that stimulate cellular migration and have well-established roles in angiogenesis and human diseases, such as cancer and atherosclerosis (Demoulin, J. B. & Essaghir, A. PDGF receptor signaling networks in normal and cancer cells. Cytokine & growth factor reviews, doi:10.1016/j.cytogfr.2014.03.003 (2014); Kohler, N. & Lipton, A. Platelets as a source of fibroblast growth-promoting activity. Erp Cell Res 87, 297-301 (1974); Ross, R., Glomset, J., Kariya, B. & Harker, L. A platelet-dependent serum factor that stimulates the proliferation of arterial smooth muscle cells in vitro. Proc Natl AcadSci U1SA 71, 1207-1210 (1974); Westermark, B. & Wasteson, A. A platelet factor stimulating human normal glial cells. Exp Cell Res 98, 170-174 (1976)). The PDGFs are encoded by four different genes and function as secreted homodimeric or heterodimeric proteins that bind to, and induce phosphorylation of the PDGF receptor (PDGFR) α and β subunits (Demoulin, J. B. & Essaghir, A. PDGF receptor signaling networks in normal and cancer cells. Cytokine & growthfactor reviews, doi:10.1016/j.cytogfr.2014.03.003 (2014)). ERBB2 is also a receptor tyrosine kinase (RTK) that forms heterodimers with other RTKs, including EGFR (epidermal growth factor receptor), to transmit growth factor-related signals (Spivak-Kroizman, T. et al. Heterodimerization of c-erbB2 with different epidermal growth factor receptor mutants elicits stimulatory or inhibitory responses. J Biol Chem 267, 8056-8063 (1992). These pathways represent viable candidates to mediate fungus-host interactions since each has a plasma membrane-bound receptor. We will focus our studies in this Aim on these three GFRs because of their potential to modulate invasion of alveolar epithelial cells by Mucorales and because of the feasibility of using them as targets for immediate noval adjunctive therapy for mucormycosis.

In vivo host transcriptomics. We performed RNA-seq analysis on the host response to R. delemar infection in the Ibrahim group's well-established murine DKA model of mucormycosis (Gebremariam, T. et al. CotH3 mediates fungal invasion of host cells during mucormycosis. J Clin Invest 124, 237-250, doi:10.1172/JCI71349 (2014); Gebremariam, T. et al. Bicarbonate correction of ketoacidosis alters host-pathogen interactions and alleviates mucormycosis. J Clin Invest, doi:10.1 172/JCI82744 (2016); Luo, G. et al. Efficacy of liposomal amphotericin B and posaconazole in intratracheal models of murine mucormycosis. Antimicrob Agents Chemother 57, 3340-3347, doi:10.1128/AAC.00313-13 (2013)). Three DKA mice were infected intratracheally with 2.5×10⁵R. delemar strain 99-880 spores (Luo, G. et al. Efficacy of liposomal amphotericin B and posaconazole in intratracheal models of murine mucormycosis. Antimicrob Agents Chemother 57, 3340-3347, doi:10.1128/AAC.00313-13 (2013)). Lungs were excised 14 h post infection. Three sham-infected DKA mice acted as a control. RNA-seq analysis was performed and the data were used to identify host pathways that are modulated in the infected mice but not in the uninfected control. For in vitro infections, most or all of the host cells in a monolayer are infected yielding a stronger signal, however this is obviously not as biologically relevant. For in vivo infections in whole infected organs, a large portion of host cells are not in contact with the pathogen, thus yielding a smaller signal from those cells in contact with the pathogen. Despite this limitation, our upstream regulator analysis indicated the activation of signaling of EGFR pathway during R. delemar infection in vivo (published by our groups, mBio, 2018) (Watkins, T. N. et al. Inhibition of EGFR Signaling Protects from Mucormycosis. MBio 9, doi:10.1128/mBio.01384-18 (2018)). Collectively, our transcriptomic results suggest that Mucorales infection in vitro and in vivo activates signaling, directly or indirectly, through PDGF receptors, EGFR and ErbB2.

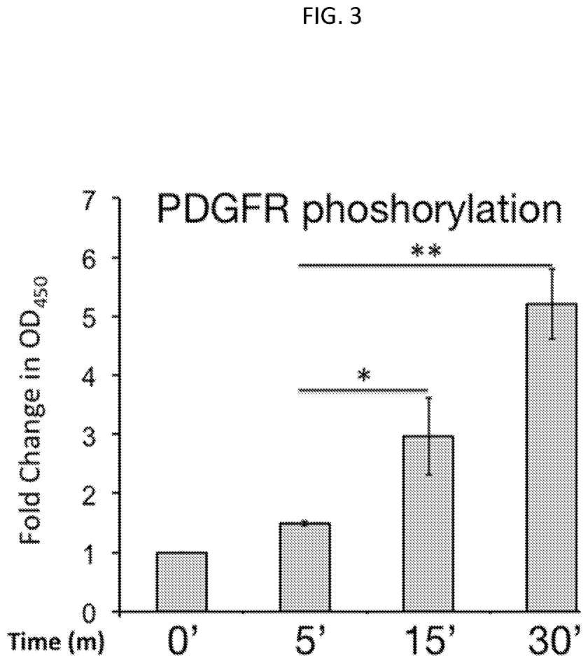

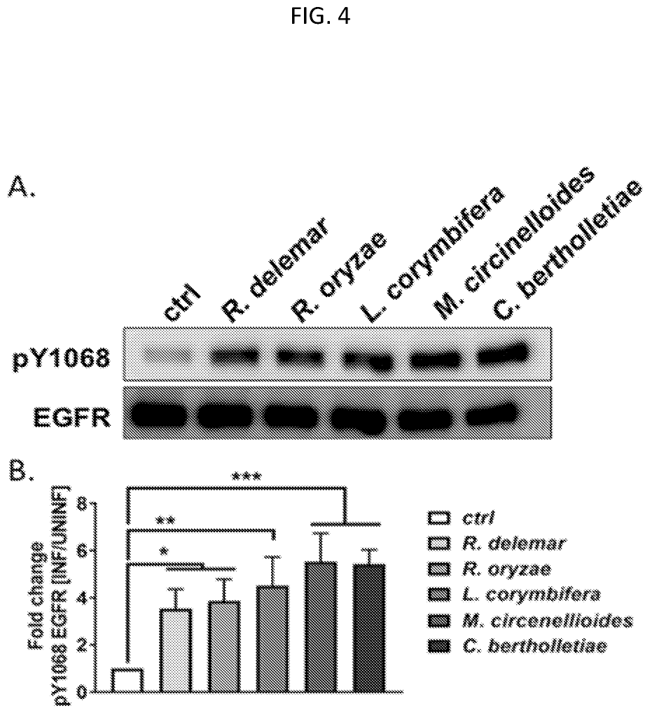

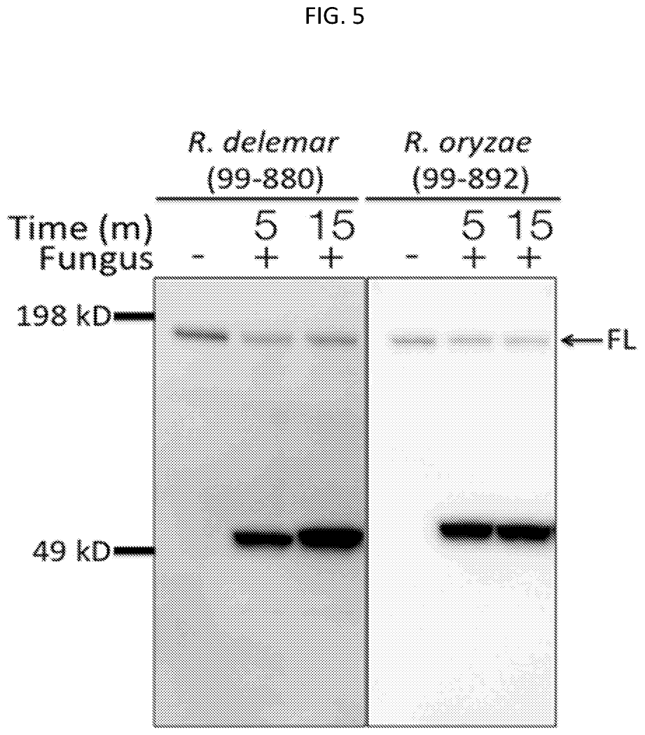

Detection of GFR activation during in vitro infection. When PDGF receptor subunits and EGFR are activated by their respective cognate ligands, they become phosphorylated on tyrosine residues at the plasma membrane (Bishayee, S., Ross, A. H., Womer, R. & Scher, C. D. Purified human platelet-derived growth factor receptor has ligand-stimulated tyrosine kinase activity. Proc Natl Acad Sci US A 83, 6756-6760 (1986); Decker, S. J. Transmembrane signaling by epidermal growth factor receptors lacking autophosphorylation sites. J Biol Chem 268, 9176-9179 (1993)). Hazan, R. et al. Identification of autophosphorylation sites of HER2/neu. Cell growth & differentiation: the molecular biology journal of the American Association for Cancer Research 1, 3-7 (1990). (Bishayee, S., Ross, A. H., Womer, R. & Scher, C. D. Purified human platelet-derived growth factor receptor has ligand-stimulated tyrosine kinase activity. Proc Natl Acad Sci USA 83, 6756-6760 (1986); Decker, S. J. Transmembrane signaling by epidermal growth factor receptors lacking autophosphorylation sites. J Biol Chem 268, 9176-9179 (1993)). These activated receptor tyrosine kinases can transmit their signals through the 3 major MAP kinase signaling pathways (i.e., ERK1/2, JNK and p38) (Hopkins, P. N. Molecular biology of atherosclerosis. Physiological reviews 93, 1317-1542, doi:10.1152/physrev.00004.2012 (2013). Consistent with this, both our in vitro and in vivo RNA-seq experiments predicted the activation of ERK and JNK signaling (data not shown). Within 30 minutes post-infection in vitro with R. delemar, there is an increase in the phosphorylation of PDGF receptor subunits (FIG. 3). We also observe an increase in the phosphorylation of and EGFR in A549 cells infected individually with 5 different species of mucormycosis-causing fungi including: R. delemar, R. oryzae, Lichtheimia corymbifera, M. circinelloides and Cunninghamella bertholettiae (FIG. 4), providing evidence that GFR activation is not a strain- or species specific phenomenon. Furthermore, we observed that the phosphorylated (activated) form of EGFR co-localized with R. delemar spores during in vitro infection of A549 cells (mBio, 2018)(Watkins, T. N. et al. Inhibition of EGFR Signaling Protects from Mucormycosis. MBio 9, doi:10.1128/mBio.01384-18 (2018)). There are no known ligands that bind to ErbB2 alone. Rather, ErbB2 forms heterodimers with EGFR and other ErbB proteins and is known to be phosphorylated and cleaved upon ligand dependent activation of the heterodimers (Wada, T., Qian, X. L. & Greene, M. I. Intermolecular association of the p185neu protein and EGF receptor modulates EGF receptor function. Cell 61, 1339-1347 (1990)). Using an antibody which recognizes the C-terminus of ErbB2, we observed a ˜50 kD ErbB2 cleavage product immediately following infection with R. delemar or R. oryzae post infection (FIG. 5). This cleavage does not occur when the fungal cells are separated from the A549 cells with a transwell membrane (data not shown). Using an antibody recognizing the N-terminus (extracellular domain) of ErbB2, we also detect significant accumulation of the shed extracellular domain in the culture supernatant following infection (data not shown).

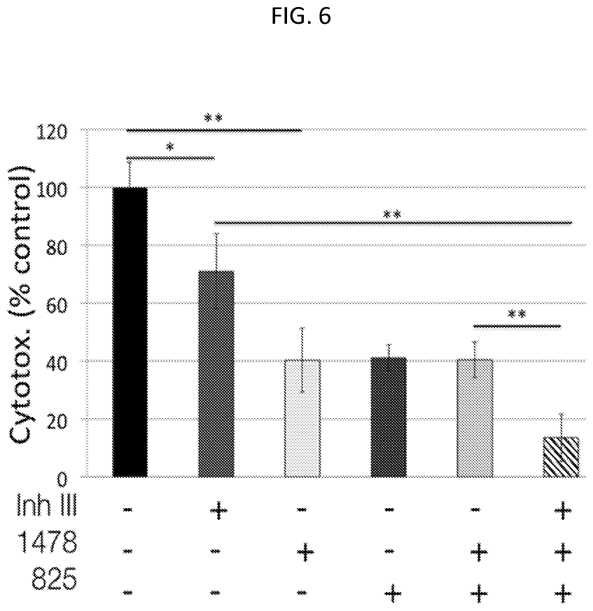

Inhibition of GFR phosphorylation inhibits damage (Bruno group). Inhibition of PDGFR phosphorylation with PDGFR tyrosine kinase inhibitor III (CAS 205254-94-0) significantly reduces R. delemar-induced damage of HUVECs (Chibucos, M. C. et al. An integrated genomic and transcriptomic survey of mucormycosis-causing fungi. Nature communications 7, 12218, doi:10.1038/ncomms12218 (2016)). Pre-treatment with inhibitor III modestly but significantly reduces R. delemar-induced damage of A549 alveolar epithelial cells as determined by LDH assay (FIG. 6). A greater reduction in damage is observed when the cells are pre-treated individually with inhibitors of either EGFR phosphorylation (AG1478) or ErbB2 phosphorylation (AG825). Co-treatment with EGFR and ErbB2 inhibitors has the same effect as each of the inhibitors individually, consistent with a model in which these proteins function in the same pathway (specifically, as a heterodimer) to transmit signals. Pre-treatment of A549 cells with all 3 inhibitors (PDGFR, EGFR and ErbB2) almost completely abrogated damage to 10% of the vehicle control (FIG. 6) suggesting that PDGFR subunits and the EGFR/ErbB2 heterodimer function independently in promoting R. delemar-induced invasion and subsequent damage. Treatment of R. delemar with the inhibitors, in the absence of host cells, did not affect growth or germination (data not shown). Our long-term goal is for us to prevent all GFR receptors involved in invasion of host epithelial cells at early stages of infection, thereby improving on mucormycosis treatment outcome.

Antibodies to the extracellular domain of EGFR and ErbB2 inhibit in vitro infection. In a complementary approach to blocking GFR phosphorylation, we tested the effect of antibodies recognizing the extracellular domains of EGFR or ErbB2, on the host pathogen interaction in vitro. Cetuximab recognizes EGFR and Trastuzumab recognizes ErbB2. Each of these antibodies is FDA-approved for cancer treatment. A significant reduction in both damage and endocytosis is observed following pre-treatment with Cetuximab or Trastuzumab (mBio, 2018, data not shown) (Watkins, T. N. et al. Inhibition of EGFR Signaling Protects from Mucormycosis. MBio 9, doi:10.1128/mBio.01384-18 (2018)). No additional decrease in damage was observed following combination treatment of both antibodies, providing further support that EGFR and ErbB2 function as a heterodimer to promote fungal invasion. Treatment of R. delemar with the antibodies, in the absence of host cells, did not affect growth or germination (Watkins, T. N. et al. Inhibition of EGFR Signaling Protects from Mucormycosis. MBio 9, doi:10.1128/mBio.01384-18 (2018)).

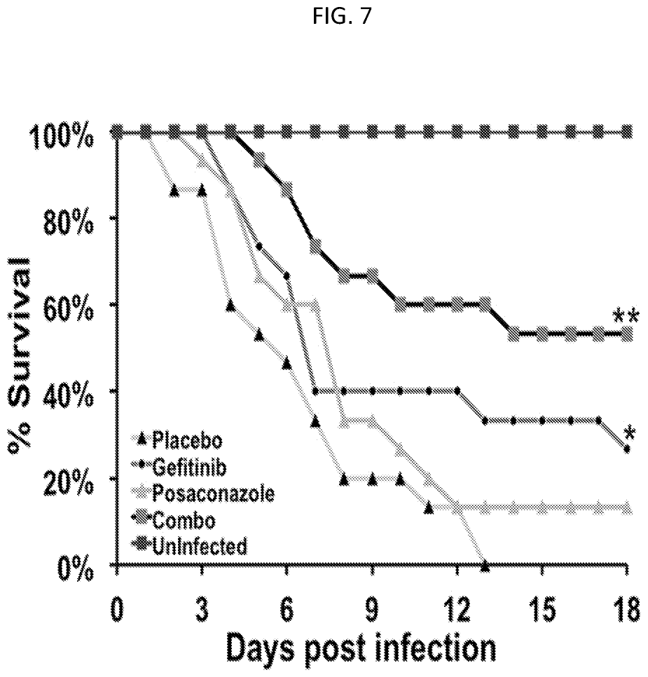

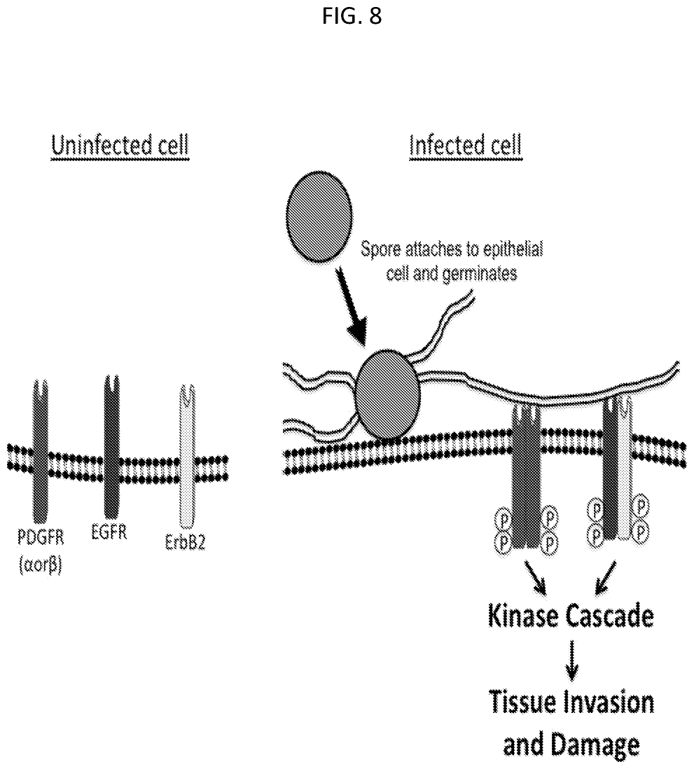

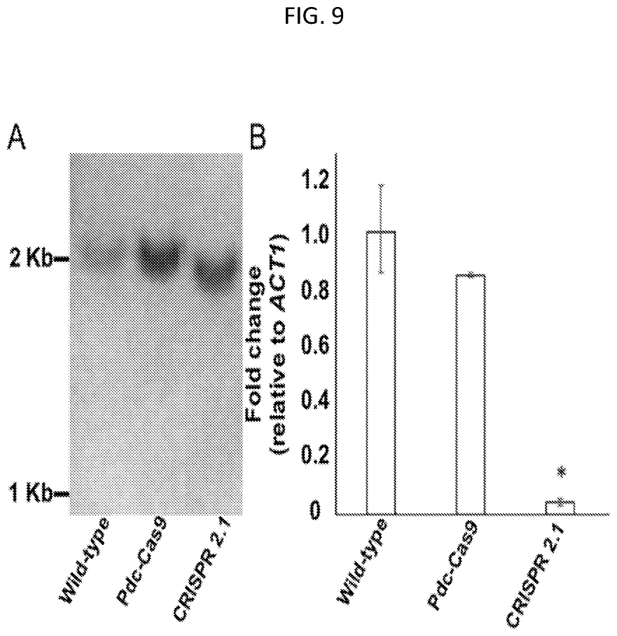

Gefitinib treatment increases survival of mice with mucormycosis. To investigate the relevance of our in vitro GFR findings, we infected neutropenic mice intratracheally with R. delemar (Luo, G. et al. Efficacy of liposomal amphotericin B and posaconazole in intratracheal models of murine mucormycosis. Antimicrob Agents Chemother 57, 3340-3347, doi:10.1128/AAC.00313-13 (2013)). and treated with 10 mg/kg of Gefitinib (a currently used EGFR inhibitor to treat cancer) (Lynch, T. J. et al. Activating mutations in the epidermal growth factor receptor underlying responsiveness of non-small-cell lung cancer to gefitinib. N Engl J Med 350, 2129-2139, doi:10.1056/NEJMoa040938 (2004)) for 5 consecutive days starting 4 h post infection since invasion of lung epithelial cells is an early event in the infection. Mice treated with gefitinib were protected from invasive pulmonary mucormycosis. Furthermore, mice treated with gefitinib had˜1 log reduction in fungal burden in their organs (lungs and brains) when compared to placebo-treated mice (mBio, 2018) (Watkins, T. N. et al. Inhibition of EGFR Signaling Protects from Mucormycosis. MBio 9, doi:10.1128/mBio.01384-18 (2018)). To demonstrate the use of this approach as treatment of late established pulmonary infection, we performed an experiment in which gefitinib administration was delayed for 30 h alone or in combination with another standardly used antifungal, posaconazole. Note that hematogenous dissemination in these intratracheally infected mice occur as early as 24 h post infection (Baldin, C. et al. PCR-based approach targeting Mucorales specific gene family for the diagnosis of mucormycosis. J Clin Microbiol, doi:10.1128/JCM.00746-18 (2018)). When administered alone, delayed treatment of gefitinib protected mice from pulmonary mucormycosis while posaconazole did not (FIG. 7, ˜30% 18-day survival for gefitinib vs. 0% for placebo or posaconazole). Further, combination treatment of gefitinib+posaconazole doubled 18-day survival of mice (˜60%) (FIG. 7). These data confirm the role of GFRs in mucormycosis pathogenesis and raise the intriguing possibility of immediately repurposing currently FDA-approved cancer drugs to treat mucormycosis both prophylactically and in established infections. Our working model is summarized in FIG. 8.

Approach. In this Aim, we will determine the specific role that the GFRs play in the interaction between alveolar epithelial cells and Mucorales (subpart IA), identify the fungal components that stimulate GFR activation (subpart 1B), and assess the contribution of GFR signaling during mucormycosis in vivo (subpart 1C). These experiments will be performed using the established alveolar epithelial cell line (A549).

1A. Examine the role of GFRs in fungal-host interactions in vitro. R. delemar-induced damage of alveolar epithelial cells, requires that the fungal cells adhere to and induce their uptake into the host cells. While our preliminary data indicate that blocking GFR signaling abrogates damage, we do not know which part of the process is blocked by the inhibitors. In this sub-aim, we will examine the role of each individual GFR in mediating R. delemar-induced invasion at each stage during the interaction (i.e. adherence, endocytosis and infection-induced damage). The necessity of each GFR will be tested by using complimentary approaches of siRNA and antibody inhibition to block the function of the receptor(s). The sufficiency of each GFR will be tested by heterologous expression in Chinese hamster ovary (CHO) cells. We will initially use R. delemar strain 99-880 for these experiments. Subsequent experiments will use other Mucorales species (R. oryzae, M. circinelloides, L. corymbifera and C. bertholletiae) to determine if the results are generalizable among Mucorales fungi.

Assaying fungal adherence to and endocytosis by A549 cells. Adhesion and endocytosis of R. delemar (strain 99-880) will be assayed using our well-established protocol (Gebremariam, T. et al. CotH3 mediates fungal invasion of host cells during mucormycosis. J Clin Invest 124, 237-250, doi:10.1172/JCI71349 (2014)). Briefly, 12-mm glass coverslips in a 24-well culture plate will be coated with fibronectin for at least 4 h and seeded with A549 alveolar epithelial cells. After washing, cells will be infected with 105 R. delemar spores. After incubation for 3 h, cells will be fixed in 3% paraformaldehyde and stained for 1 h with 1% Uvitex, which specifically binds to fungal cell wall chitin. After washing 3 times with PBS, coverslips will be mounted on a glass slide with a drop of ProLong Gold anti-fade reagent (Molecular Probes) and sealed. The total number of cell-associated organisms (i.e., spores adhering to the monolayer) per high-powered field will be determined by phase-contrast microscopy. The same field will be examined by epifluorescence microscopy, and the number of brightly-fluorescing, uninternalized germlings will be determined. The number of endocytosed organisms is calculated by subtracting the number of fluorescent organisms from the total number of visible organisms. At least 400 organisms will be counted in 20-40 different fields per slide. Experiments will be performed in triplicate on 3 separate days for a minimum of nine replicates. The counting will be done blindly.

Assaying fungal induced-damage of host cells. A549 cell damage following infection with R. delemar will be quantified using our established Cr release assay (Gebremariam, T. et al. CotH3 mediates fungal invasion of host cells during mucormycosis. J Clin Invest 124, 237-250, doi:10.1172/JCI71349 (2014)). Cells grown in 96-well tissue culture plates containing detachable wells will be incubated with 1 μCi/well Na₂⁵¹CrO₄ (ICN) with A549 cells for 16 h. On the day of the experiment, the unincorporated ⁵¹Cr will be aspirated, and wells will be washed twice with prewarmed HBSS. Cells will be infected with fungal cells (1.5×105 spores) suspended in 150 μl RPMI 1640 medium supplemented with glutamine. Spontaneous ⁵¹Cr release will be determined by incubating A549 cells in RPMI 1640 medium supplemented with glutamine without R. delemar.

After 24 h of incubation at 37° C. in a 5% CO₂ incubator, 50% of the medium will be aspirated from each well and transferred to glass tubes, and wells will be manually detached and placed into another set of tubes. The amount of ⁵¹Cr in the aspirate and the detached wells will be determined by gamma counting. The total amount of ⁵¹Cr incorporated by endothelial cells in each well will be calculated as the sum of radioactive counts per minute of the aspirated medium and radioactive counts of the corresponding detached wells. After correcting the data for variations in the amount of tracer incorporated in each well, the percentage of specific endothelial cell release of ⁵¹Cr will be calculated as follows: [(experimental release×2)−(spontaneous release×2)]/[total incorporation˜(spontaneous release×2)]. Each experimental condition will be tested at least in triplicate, and the experiment repeated twice for a total of 3 independent experiments.

Antibody inhibition. We will also ascertain whether antibodies directed against each candidate GFRs (PDGFRα, PDGFRβ, EGFR and ErbB2) can block alveolar epithelial cell adhesion, invasion and subsequent damage by R. delemar. In these experiments, control epithelial cells will be incubated with an irrelevant isotype-matched antibody in parallel (Fu, Y. et al. Candida albicans Als1p: an adhesin that is a downstream effector of the EFG1 filamentation pathway. Mol Microbiol 44, 61-72 (2002); Liu, M. et al. The endothelial cell receptor GRP78 is required for mucormycosis pathogenesis in diabetic mice. J Clin Invest 120, 1914-1924, doi:42164 [pii]10.1172/JCI42164). Antibodies targeting PDGFR subunits, EGFR and ErbB2 are available commercially and will be used for these studies. Polyclonal Abs are preferable to monoclonal mAbs for these blocking experiments, because a mAb that does not recognize the binding region of the GFR may not affect the assay tested. In addition, blocking experiments using exogenous recombinant GFRs will also be performed, if they are available commercially. For those proteins which are not available commercially, we will use our decades' long expertise in generating His-, FLAG, or Halo-tagged recombinant proteins by expressing them in E. coli or S. cerevisiae (Fu, Y. et al. Candida albicans Als1p: an adhesin that is a downstream effector of the EFG1 filamentation pathway. Mol Microbiol 44, 61-72 (2002); Liu, M. et al. Fob1 and Fob2 Proteins Are Virulence Determinants of Rhizopus oryzae via Facilitating Iron Uptake from Ferrioxamine. PLoS Pathog 11, e1004842, doi:10.1371/journal.ppat.1004842 (2015); Ibrahim, A. S. et al. The high affinity iron permease is a key virulence factor required for Rhizopus oryzae pathogenesis. Mol Microbiol 77, 587-604, doi:10.1111/j.1365-2958.2010.07234.x (2010); Luo, G., Ibrahim, A. S., French, S. W., Edwards Jr., J. E. & Fu, Y. Active and Passive Immunization with rHyrlp-N Protects Mice against Hematogenously Disseminated Candidiasis. PLoS One 6, e25909, doi:doi:10.1371/journal.pone.0025909 (2011)). The expressed proteins will be purified by affinity purification followed by cleaving of the tag. The identity of the expressed proteins (i.e. PDGFR, EGFR, and ErB2) will be determined by Western blotting assays using antibodies targeting these proteins, followed by sequencing the purified proteins by LC/MS. Experiments will be conducted in triplicate and repeated at least twice.

siRNA knockdown of GFRs. We will attenuate GFR expression using siRNA. Alveolar epithelial cells will be transfected with either GFR siRNA or an irrelevant control siRNA (Qiagen), following our published procedures (Liu, M. et al. The endothelial cell receptor GRP78 is required for mucormycosis pathogenesis in diabetic mice. J Clin Invest 120, 1914-1924, doi:42164 [pii]10.1172/JCI42164). The extent of GFR attenuation will be assessed by RT-PCR, immunoblotting of total cell lysates with the anti-GFR antibody, as well as determining the amount of expression on the cell surface by flow cytometry (Gebremariam, T. et al. Bicarbonate correction of ketoacidosis alters host-pathogen interactions and alleviates mucormycosis. J Clin Invest, doi:10.1172/JCI82744 (2016); Liu, M. et al. Fob1 and Fob2 Proteins Are Virulence Determinants of Rhizopus oryzae via Facilitating Iron Uptake from Ferrioxamine. PLoS Pathog 11, e1004842, doi:10.1371/journal.ppat.1004842 (2015); Al-Bader, N. et al. Role of trehalose biosynthesis in Aspergillus fumigatus development, stress response, and virulence. Infect Immun 78, 3007-3018, doi:IAI.00813-09 [pii] 10.1128/IAI.00813-09). The effects of GFR attenuation on the R. delemar adherence to, endocytosis by, and damage of A549 alveolar epithelial cells will then be determined as above. Each GFR expression will be attenuated individually or in combination with other GFRs to determine the GFR function together or separately. The GFRs to be targeted will be: PDGFRα, PDGFRβ, EGFR and ErbB2. As necessary, we will use CRISPR/CAS9 to delete selected GFR from the human cells lines using standard techniques.

Heterologous expression of GFRs. To ascertain whether expression of a GFR is sufficient to mediate host cell invasion, we will transfect a mammalian cell line with the corresponding GFR gene, as we did previously (Liu, M. et al. The endothelial cell receptor GRP78 is required for mucormycosis pathogenesis in diabetic mice. J Clin Invest 120, 1914-1924, doi:42164 [pii]10.1172/JCI42164). We will clone the full-length GFR gene from epithelial cells by RT-PCR. The sequence-verified GFR cDNA will then be cloned into the pcDNA3.1 mammalian expression vector (Invitrogen) to create a pcDNA3.1-GFR plasmid. We will transfect a mammalian cell line, CHO K-I cells, with pcDNA3.1-ECR to create a GFR-overexpressing cell line. Clones will be screened for surface expression of GFR by flow cytometry (Liu, M. et al. The endothelial cell receptor GRP78 is required for mucormycosis pathogenesis in diabetic mice. J Clin Invest 120, 1914-1924, doi:42164 [pii]10.1172/JCI42164). CHO cells expressing each of the GFR will be used in the interaction assays (i.e. adhesion, invasion and damage) as detailed above.

Immunofluorescent confocal microscopy. To determine if a given GFR co-localizes with Mucorales cells during endothelial cell endocytosis, we will use confocal microscopic imaging as we have done previously (Gebremariam, T. et al. CotH3 mediates fungal invasion of host cells during mucormycosis. J Clin Invest 124, 237-250, doi:10.1172/JCI71349 (2014); Liu, M. et al. The endothelial cell receptor GRP78 is required for mucormycosis pathogenesis in diabetic mice. J Clin Invest 120, 1914-1924, doi:42164 [pii]10.1172/JCI42164). A549 cells will be infected with R. delemar (strain 99-880), fixed, and then stained with an anti-GFR antibody. The organisms will be visualized by differential interference contrast, and the ones that are in the process of being endocytosed will be identified by staining the cells with Alexa 568-phalloidin, which labels the host actin filaments that accumulate around the invading fungus. Time course studies will be performed to see if the GFR accumulates around organisms when they first adhere or as they are being endocytosed.

1B. Identify fungal ligand(s) that bind to host GFRs. Once we have determined which of the GFRs functions as a receptor on alveolar epithelial cells, we will conduct FAR-Western blotting on proteins collected from supernatants of R. delemar regenerated protoplasts, with the host receptor as bait (Liu, M. et al. Fob1 and Fob2 Proteins Are Virulence Determinants of Rhizopus oryzae via Facilitating Iron Uptake from Ferrioxamine. PLoS Pathog 11, e1004842, doi:10.1371/journal.ppat.1004842 (2015)). During the early stages of this regeneration process, surface protein precursors are shed into the extracellular medium, but not yet covalently incorporated into the nascent cell surface, thereby enabling their easy isolation and identification (Pitarch, A., Nombela, C. & Gil, C. Collection of proteins secreted from yeast protoplasts in active cell wall regeneration. Methods Mol Biol425, 241-263, doi:10.1007/978-1-60327-210-0_20 (2008)). We have successfully employed this technique to identify CotH3 (Gebremariam, T. et al. CotH3 mediates fungal invasion of host cells during mucormycosis. J Clin Invest 124, 237-250, doi:71349 [pii] 10.1172/JCI71349) and ferrioxamine receptors (Liu, M. et al. Fob1 and Fob2 Proteins Are Virulence Determinants of Rhizopus oryzae via Facilitating Iron Uptake from Ferrioxamine. PLoS Pathog 11, e1004842, doi:10.1371/journal.ppat.1004842 (2015)), The recognized bands on the Western blot will be identified by LC/MS and each of the identified fungal putative ORFs will be expressed in S. cerevisiae, which does not adhere to or invade alveolar epithelial cells (Sheppard, D. C. et al. Functional and structural diversity in the Als protein family of Candida albicans. J Biol Chem 279, 30480-30489 (2004); Fu, Y. et al. Expression of the Candida albicans gene ALS1 in Saccharomyces cerevisiae induces adherence to endothelial and epithelial cells. Infect Immun 66, 1783-1786. (1998), by cloning from a cDNA library and expression confirmed with qRT-PCR. This heterologous expression system was used successfully by us to identify CotH3 as a ligand to GRP78 in HUVEC (Gebremariam, T. et al. CotH3 mediates fungal invasion of host cells during mucormycosis. J Clin Invest 124, 237-250, doi:71349 [pii] 10.1172/JCI71349). Adherence and invasion of: 1) epithelial cells; 2) epithelial cells with reduced GFR expression (by siRNA lentivirus) (Liu, M. et al. The endothelial cell receptor GRP78 is required for mucormycosis pathogenesis in diabetic mice. J Clin Invest 120, 1914-1924, doi:42164 [pii]10.1172/JCI42164); 3) CHO cells that overexpress the receptor (Liu, M. et al. The endothelial cell receptor GRP78 is required for mucormycosis pathogenesis in diabetic mice. J Clin Invest 120, 1914-1924, doi:42164 [pii]10.1172/JCI42164); or wild-type CHO cells will be compared after incubation with S. cerevisiae expressing the ORF (Gebremariam, T. et al. CotH3 mediates fungal invasion of host cells during mucormycosis. J Clin Invest 124, 237-250, doi:71349 [pii] 10.1172/JCI71349). Cell association of S. cerevisiae with an empty plasmid will be used as a control. Experiments will be conducted in triplicate, repeated on three different days, and the results acquired blindly.

To demonstrate the specificity of the attachment, the binding of the ligand to its receptor will be quantitatively tested by using Biacore T-100 at UCLA core facility (Ibrahim Lab) (Adams, E. L. et al. Differential high-affinity interaction of dectin-1 with natural or synthetic glucans is dependent upon primary structure and is influenced by polymer chain length and side-chain branching. J Pharmacol Exp Ther 325, 115-123, doi:jpet.107.133124 [pii]10.1124/jpet.107.133124 (2008); Kougias, P. et al. Normal human fibroblasts express pattern recognition receptors for fungal (1- ->3)-beta-D-glucans. Infect Immun 69, 3933-3938, doi:10.1128/IAI.69.6.3933-3938.2001 (2001); Rice, P. J. et al. Human monocyte scavenger receptors are pattern recognition receptors for (1- ->3)-beta-D-glucans. J Leukoc Biol 72, 140-146 (2002)). The recombinant GFR will be immobilized on sensor chips and increasing concentrations of recombinantly produced ligand will be used as the analyte and compared to results with BSA.

1C. Explore the therapeutic potential of targeting GFRs in vivo to treat mucormycosis. Mice harboring deletions in PDGFRα, PDGFRβ, EFGR or ErbB2 die either before birth or within the first week of life (Lee, K. F. et al. Requirement for neuregulin receptor erbB2 in neural and cardiac development. Nature 378, 394-398, doi:10.1038/378394a0 (1995); Miettinen, P. J. et al. Epithelial immaturity and multiorgan failure in mice lacking epidermal growth factor receptor. Nature 376, 337-341, doi:10.1038/376337a0 (1995); Soriano, P. Abnormal kidney development and hematological disorders in PDGF beta-receptor mutant mice. Genes Dev 8, 1888-1896 (1994); Soriano, P. The PDGF alpha receptor is required for neural crest cell development and for normal patterning of the somites. Development 124, 2691-2700 (1997)), thus precluding our ability to test the receptors in traditional mouse gene deletion experiments. Further, there are no lung-specific deletion models for any of our GFRs. Our preliminary data indicate that pharmacological inhibition of the GFRs significantly reduces Rhizopus-induced damage of alveolar epithelial cells with the largest effect coming from the combinatorial inhibition of all three. Additionally, and as a proof-of-concept, we show that the EGFR inhibitor, gefitinib, protected neutropenic mice from R. delemar infection when given 4 h post infection. In this sub-Aim, we will determine if the pharmacological inhibition of GFR can protect against murine mucormycosis when given in a more established infection model (e.g. 24 h post infection) since mucormycosis is often diagnosed late. We will also compare the efficacy of different GFR inhibitors to currently used first line therapy of liposomal amphotericin B (LAmB). Finally, because these GFR are likely to be used as adjunctive therapy, we will determine if combining these GFR with LAmB will constitute an improvement over monotherapy.

In vivo model. We will evaluate the effect of various GFR tyrosine kinase inhibitors using the Ibrahim's laboratory clinically relevant neutropenic mouse model of pulmonary mucormycosis (Luo, G. et al. Efficacy of liposomal amphotericin B and posaconazole in intratracheal models of murine mucormycosis. Antimicrob Agents Chemother 57, 3340-3347, doi:10.1128/AAC.00313-13 (2013); Gebremariam, T. et al. VT-1161 Protects Immunosuppressed Mice from Rhizopus arrhizus var. arrhizus Infection. Antimicrob Agents Chemother 59, 7815-7817, doi:10.1128/AAC.01437-15 (2015); Luo, G. et al. Isavuconazole therapy protects immunosuppressed mice from mucormycosis. Antimicrob Agents Chemother 58, 2450-2453, doi:10.1128/AAC.02301-13 (2014)). Cyclophosphamide/cortisone acetate-induced neutropenic mice will be inoculated intratracheally with 2.5×10⁵ spores (Luo, G. et al. Efficacy of liposomal amphotericin B and posaconazole in intratracheal models of murine mucormycosis. Antimicrob Agents Chemother 57, 3340-3347, doi:10.1128/AAC.00313-13 (2013); Gebremariam, T. et al. VT-1161 Protects Immunosuppressed Mice from Rhizopus arrhizus var. arrhizus Infection. Antimicrob Agents Chemother 59, 7815-7817, doi:10.1128/AAC.01437-15 (2015); Luo, G. et al. Isavuconazole therapy protects immunosuppressed mice from mucormycosis. Antimicrob Agents Chemother 58, 2450-2453, doi:10.1128/AAC.02301-13 (2014)), an inoculum consistently delivers ˜1-5×10³ spores to the lungs representing a direct interaction with alveolar epithelial cells (Ibrahim, A. S. et al. Caspofungin inhibits Rhizopus oryzae 1,3-beta-D-glucan synthase, lowers burden in brain measured by quantitative PCR, and improves survival at a low but not a high dose during murine disseminated zygomycosis. Antimicrob Agents Chemother 49, 721-727, doi:10.1128/AAC.49.2.721-727.2005 (2005); Ibrahim, A. S. et al. The iron chelator deferasirox protects mice from mucormycosis through iron starvation. J Clin Invest 117, 2649-2657, doi:10.1172/JCI32338 (2007); Ibrahim, A. S., Gebremariam, T., Fu, Y., Edwards, J. E., Jr. & Spellberg, B. Combination echinocandin-polyene treatment of murine mucormycosis. Antimicrob Agents Chemother 52, 1556-1558, doi:10.1128/AAC.01458-07 (2008); Luo, G. et al. Efficacy of liposomal amphotericin B and posaconazole in intratracheal models of murine mucormycosis. Antimicrob Agents Chemother 57, 3340-3347, doi:AAC.00313-13 [pii] 10.1128/AAC.00313-13). For each of the 3 GFRs discussed there exists a targeted, FDA approved tyrosine kinase inhibitor that is currently being used in the clinic for the treatment of cancer.

Imatinib (Gleevec/STI-571) is a potent inhibitor of PDGFR tyrosine phosphorylation. Lapatinib (Tykerb) is a potent inhibitor that targets the ATP binding pocket of both EGFR and ErbB2 (Gril, B. et al. Effect of lapatinib on the outgrowth of metastatic breast cancer cells to the brain. Journal of the National Cancer Institute 100, 1092-1103, doi:10.1093/jnci/djn216 (2008)). Gefitinib (Iressa) is a tyrosine kinase inhibitor specific for EGFR (Lynch, T. J. et al. Activating mutations in the epidermal growth factor receptor underlying responsiveness of non-small-cell lung cancer to gefitinib. N Engl J Med 350, 2129-2139, doi:10.1056/NEJMoa040938 (2004)). Each of these inhibitors is commercially available, have demonstrated efficacy in mice and will be tested in our model. Moreover, the ability of GW2974, an orally-bioavailable dual EGFR and ErbB2 tyrosine kinase inhibitor will be also tested. All drugs will be administered by oral gavage. Imatinib will be administered twice daily at 25 mg/kg (Hoepfl, J. et al. Effects of imatinib on bone marrow engrafiment in syngeneic mice. Leukemia 16, 1584-1588, doi:10.1038/sj.leu.2402679 (2002)). Lapatinib will be given at 100 mg/kg twice daily (Gril, B. et al. Effect of lapatinib on the outgrowth of metastatic breast cancer cells to the brain. Journal of the National Cancer Institute 100, 1092-1103, doi:10.1093/jnci/djn216 (2008)) while gefinitib and GW2974 will be given at 10 mg/kg/qd (FIG. 8) and 30 mg/kg/qd, respectively. Treatment will start on Day+1 and continued until Day+10 relative to infection (Wang, L. et al. Differential effects of low- and high-dose GW2974, a dual epidermal growth factor receptor and HER2 kinase inhibitor, on glioblastoma multiforme invasion. Journal of Neuroscience Research 91, 128-137, doi:10.1002/jnr.23140 (2013)). As a control arm, mice will be infected as above and treated with LAmB (15 mg/kg/qd, given via tail vein) (Luo, G. et al. Efficacy of liposomal amphotericin B and posaconazole in intratracheal models of murine mucormycosis. Antimicrob Agents Chemother 57, 3340-3347, doi:10.1128/AAC.00313-13 (2013)). Finally, since we expect redundancy in signaling pathways that govern Mucorales invasion, and have provided evidence for this in FIG. 6, each of the GFR-targeting molecules will be tested in combination with each other and LAmB to determine the best combinatorial therapy that will constitute an improvement over administration of single drugs alone. Our primary endpoint of efficacy will be time to euthanasia of moribund mice (Luo, G. et al. Efficacy of liposomal amphotericin B and posaconazole in intratracheal models of murine mucormycosis. Antimicrob Agents Chemother 57, 3340-3347, doi:10.1128/AAC.00313-13 (2013); Gebremariam, T. et al. VT-1161 Protects Immunosuppressed Mice from Rhizopus arrhizus var. arrhizus Infection. Antimicrob Agents Chemother 59, 7815-7817, doi:10.1128/AAC.01437-15 (2015); Luo, G. et al. Isavuconazole therapy protects immunosuppressed mice from mucormycosis. Antimicrob Agents Chemother 58, 2450-2453, doi:10.1128/AAC.02301-13 (2014)). As a secondary endpoint we will assess the tissue fungal burden of lungs and brains (primary and secondary target organs, respectively) by qPCR of mice sacrificed at selected time intervals (decided from the survival curve which will represent early, mid, and late stages of infection) (Ibrahim, A. S. et al. The iron chelator deferasirox protects mice from mucormycosis through iron starvation. J Clin Invest 117, 2649-2657, doi:10.1172/JCI32338 (2007); Ibrahim, A. S., Spellberg, B., Avanessian, V., Fu, Y. & Edwards, J. E., Jr. Rhizopus oryzae adheres to, is phagocytosed by, and damages endothelial cells in vitro. Infect Immun 73, 778-783, doi:73/2/778 [pii]10.1128/IA.73.2.778-783.2005 (2005)).

2: To identify fungal targets and determine their therapeutic potential to treat mucormycosis.

Background and Rationale. Currently, there are only 5 R. delemar-encoded genes with a demonstrated role in pathogenesis of mucormycosis (Gebremariam, T. et al. CotH3 mediates fungal invasion of host cells during mucormycosis. J Clin Invest 124, 237-250, doi:10.1172/JCI71349 (2014); Liu, M. et al. Fob1 and Fob2 Proteins Are Virulence Determinants of Rhizopus oryzae via Facilitating Iron Uptake from Ferrioxamine. PLoS Pathog 11, e1004842, doi:10.1371/journal.ppat.1004842 (2015); Ibrahim, A. S. et al. The high affinity iron permease is a key virulence factor required for Rhizopus oryzae pathogenesis. Mol Microbiol 77, 587-604, doi:10.1111/j.1365-2958.2010.07234.x (2010)) owing primarily to a historic lack of genomic information for mucormycosis-causing fungi. The Bruno lab has made some headway towards closing this gap by sequencing 36 new Mucorales genomes (Chibucos, M. C. et al. An integrated genomic and transcriptomic survey of mucormycosis-causing fungi. Nature communications 7, 12218, doi:10.1038/ncommsl2218 (2016).) and conducted detailed comparative bioinformatic analysis of these strains, representing 23 different species (Chibucos, M. C. et al. An integrated genomic and transcriptomic survey of mucormycosis-causing fungi. Nature communications 7, 12218, doi:10.1038/ncomms12218 (2016)). We have analyzed the fungal and host transcriptional response of R. oryzae and R. delemar with alveolar epithelial cells⁶ during in vitro infection. These bioinformatic and transcriptomic analyses have revealed several candidates for virulence genes that need further characterization. In this Specific Aim, we propose to further address this enormous gap in knowledge of Mucorales genes function by identifying putative virulence factors using transcriptomics of R. delemar in vivo gene expression during infection of infected lung samples. We will also study the fungal ligands identified from Aim 1B for their virulence potential by generating gene knockdown and/or knockout mutants and compare the functionality of generated mutants to wild-type strains for their virulence in vitro and in vivo. We will then test some specific hypotheses drawn from our published⁶ and preliminary data as well as the in vivo RNA-seq data to be generated in Aim 2A.

### Data

Dual-species Transcriptomics of in vitro infections. To understand the early mechanisms of alveolar epithelial cell infection (i.e. invasion and subsequent damage) by Mucorales, we identified a number of Mucorales genes that were highly expressed or whose expression was highly induced in the presence of host cells in vitro that will be the focus of Aim 2B and 2C (Chibucos, M. C. et al. An integrated genomic and transcriptomic survey of mucormycosis-causing fungi. Nature communications 7, 12218, doi:10.1038/ncomms12218 (2016)).

Selective enrichment of mold RNAs from infected mouse tissue for in vivo transcriptome profiling (Bruno group). RNA-seq on un-enriched total RNA from infected tissues is not suitable to assay the entire transcriptome of fungal pathogens (Bruno, V. M. et al. Transcriptomic analysis of vulvovaginal candidiasis identifies a role for the NLRP3 inflammasome. MBio 6, doi:10.1128/mBio.00182-15 (2015); Liu, Y. et al. New signaling pathways govern the host response to C. albicans infection in various niches. Genome Res 25, 679-689, doi:10.1101/gr.187427.114 (2015)). However, in vivo transcriptome profiling of fungal pathogens is possible after selective enrichment for fungal transcripts using the Agilent SureSelect method, as demonstrated in a murine model of candidiasis (Amorim-Vaz, S. et al. RNA Enrichment Method for Quantitative Transcriptional Analysis of Pathogens In Vivo Applied to the Fungus Candida albicans. MBio 6, e00942-00915, doi:10.1128/mBio.00942-15 (2015)). To demonstrate that this capability is effective to perform in vivo transcriptome profiling of mucormycosis-causing pathogens during infection, we tested this technique an on R. delemar.

In collaboration with the Ibrahim laboratory (Harbor-UCLA), the Bruno lab extracted total RNA from the lungs of 3 mice infected with R. delemar 99-880 via intratracheal instillation in the Ibrahim's lab DKA model of mucormycosis. A total of 218,035 non-overlapping 120-nt bait probes covering 12,932 genes used to capture the fungal component of polyA-selected libraries that were sequenced with 150-bp paired end reads on an Illumina Hiseq 4000 platform. Comparison to the un-enriched polyA-selected samples indicated a >1,660-fold enrichment following hybridization to the oligo capture library (data not shown). This level of enrichment demonstrates a cost effective method for in vivo transcriptomics, which measures the biologically relevant transcriptome.

The idea that the most highly expressed genes in the genome are good candidates for virulence genes has been validated by us and others while studying candidiasis (Bruno, V. M. et al. Transcriptomic analysis of vulvovaginal candidiasis identifies a role for the NLRP3 inflammasome. MBio 6, doi:10.1128/mBio.00182-15 (2015); Xu, W. et al. Activation and alliance of regulatory pathways in C. albicans during mammalian infection. PLoS Biol 13, e1002076, doi:10.1371/journal.pbio.1002076 (2015)). Towards this end, we noticed that the most highly in vivo expressed R. delemar gene in our experiment (IGS-99-880_09985) encodes an endo-1,3-β-D-glucanase with a secretion signal. A secreted ortholog of this protein, Engl, is required for full virulence of the fungal pathogen Histoplasma capsulatum (Garfoot, A. L., Shen, Q., Wuthrich, M., Klein, B. S. & Rappleye, C. A. The Engl beta-Glucanase Enhances Histoplasma Virulence by Reducing beta-Glucan Exposure. MBio 7, e01388-01315, doi:10.1128/mBio.01388-15 (2016)). Our hypothesis is that Mucorales can modulate the host immune response by using an endoglucanase to alter its cell wall and subsequent recognition by innate immune cells.

Approach. In our preliminary data we used transcriptomics data to develop a hypothesis that R. delemar encodes an endo-1,3-β-D-glucanase with a secretion signal, which is an ortholog to a Histoplasma virulence factor, that is required for virulence. In this Aim, we will identify virulence factors by analyzing fungal gene expression in lungs of Rhizopus-infected neutropenic mice (subpart 2A). Following a gene prioritization scheme (listed below) which considers RNA-seq data (in vivo and in vitro) as well as comparative genomics, we will generate R. delemar strains with reduced expression of each gene and test each strain's ability to invade and induce GFR signaling into alveolar epithelial cells (subpart 2B). We will determine the role of 10-20 R. delemar genes in the interaction with alveolar epithelial cells. Any mutant strains that are defective in invasion and/or GFR signaling will be tested for virulence in the neutropenic mouse model (subpart 2C). For genes whose deletions have more profound phenotypes and orthologs are present in other Mucorales genomes, mutants will be constructed and tested in R. oryzae, M. circinelloides, and L. corymbifera strains to determine how broadly applicable our results are to Mucorales (subpart 2C). This discovery-based research will vastly increase our understanding of virulence factors contributing to this devastating disease. Virulence genes that are known to be secreted or cell surface exposed will be used to develop blocking antibodies. If known inhibitors are available, they will be used to see if they protect mice from infection

Prioritization of genes for functional analysis. We will use several criteria to prioritize the fungal and host genes in this Aim. Our objective is to gain new relevant information with the minimal number of mutants, siRNAs, and mice. Our specific prioritization scheme starting with the highest priority category, is listed below. Genes that fall into more than one category will be given the highest priority ranking.

1) Genes highly expressed during murine lung infection from subpart 2A. In vivo infection studies with C. albicans, including one of ours, have suggested a correlation between gene expression and involvement in virulence without consideration for whether a gene's expression is induced by interaction with the host cell (Bruno, V. M. et al. Transcriptomic analysis of vulvovaginal candidiasis identifies a role for the NLRP3 inflammasome. MBio 6, doi:10.1128/mBio.00182-15 (2015); Xu, W. et al. Activation and alliance of regulatory pathways in C. albicans during mammalian infection. PLoS Biol 13, e1002076, doi:10.1371/journal.pbio.1002076 (2015)).

2) Genes that are either highly expressed or highly up-regulated during both in vitro and in vivo infection, based on our published⁶ and unpublished RNA-seq data of infections.

3) Genes with orthologs in the most clinically relevant Mucorales (e.g. Rhizopus, Mucor, Lichtheimia, Rhizomucor, Cunninghamella, and apophysomyces) that we have tested, but that are not conserved in mammalian cells. These Mucorales fungi cover >98% of all causes of mucormycosis thereby providing a higher potential for an effective drug design against the disease.

4) Genes that encode proteins likely to be secreted or located on the cell surface (based on the presence of secretion signal or GPI-anchor motif). These genes are high priority for investigation because they are accessible to external inhibitors, and thus have good potential as vaccine and antifungal drug targets. In addition, they will be extremely useful for identifying the host cell targets (e.g., receptors such as EGFR in 1) for these fungi.

5) Genes that encode transcription factor orthologs that govern virulence and act downstream of other regulators. The transcription factors that regulate virulence may integrate inputs from many different signaling pathways, including some that are not readily assayed in vitro. Therefore, such transcription factor genes may be essential for virulence even if other upstream pathway members are not.

6) Fungal ligands for the GFR that we identify biochemically in Subpart 1B. Others considerations:

Housekeeping genes (actin, tubulin, ribosomal proteins etc.) will be excluded from functional analyses.

If >20 genes meeting the about criteria are identified, we will prioritize based on levels of in vivo expression. The most highly expressed will be tested first.

2A. Identification of Virulence Factors using in vivo RNA-seq. In our preliminary data, we have demonstrated our ability to use transcriptomics to identify putative virulence factors using in vitro data and the abysmally small number of reads in the in vivo data. While the in vitro system can be an excellent model, understanding virulence factors in vivo is ideal and is now possible using the Agilent SureSelect as demonstrate by us (Prelim. Data) and others (Amorim-Vaz, S. et al. RNA Enrichment Method for Quantitative Transcriptional Analysis of Pathogens In Vivo Applied to the Fungus Candida albicans. MBio 6, e00942-00915, doi:10.1128/mBio.00942-15 (2015)).

Animal model. Experiments will be carried out using the Ibrahim lab's well-established neutropenic mouse model, since pulmonary mucormycosis is the major manifestation of the disease in neutropenic patients (Luo, G. et al. Efficacy of liposomal amphotericin B and posaconazole in intratracheal models of murine mucormycosis. Antimicrob Agents Chemother 57, 3340-3347, doi:10.1128/AAC.00313-13 (2013); Gebremariam, T. et al. VT-1161 Protects Immunosuppressed Mice from Rhizopus arrhizus var. arrhizus Infection. Antimicrob Agents Chemother 59, 7815-7817, doi:10.1128/AAC.01437-15 (2015)). Luo, G. et al. Isavuconazole therapy protects immunosuppressed mice from mucormycosis. Antimicrob Agents Chemother 58, 2450-2453, doi:10.1128/AAC.02301-13 (2014)). Neutropenia will be induced and the infections will be carried out as described above in subpart 1C. Total RNA will be extracted from the lungs from 3 different neutropenic mice at 1, 3 and 6 dpi (n=9). These time-points represent the various stages of disease progression.

RNA-seq of R. delemar in in vivo samples. PolyA-selected libraries will be constructed and Rhizopus transcripts enriched using Agilent SureSelect baits designed, using the publicly available eArray software (Agilent Technologies), against our recently published genome re-annotation of the R. delemar strain 99-880 (Chibucos, M. C. et al. An integrated genomic and transcriptomic survey of mucormycosis-causing fungi. Nature communications 7, 12218, doi:10.1038/ncomms12218 (2016)). Libraries will be sequenced on the Illumina HiSeq platform to obtain 5-10 million R. delemar-mapped 150-bp paired end reads, representing 35-70X transcriptome coverage. The sequencing reads will be aligned to the R. delemar strain 99-880 reference genome using TopHat, followed by HTSEQ to generate read counts and RPKM values for each gene (Liu, Y. et al. New signaling pathways govern the host response to C. albicans infection in various niches. Genome Res 25, 679-689, doi:10.1101/gr.187427.114 (2015); Bruno, V. M. et al. Transcriptomic analysis of vulvovaginal candidiasis identifies a role for the NLRP3 inflammasome. MBio 6, 00182-00115 (2015)).

Differential expression analysis. We will identify differential gene expression by comparing the expression of genes in the lungs of neutropenic mice to our published RNA-seq data gathered from R. delemar 99-880 grown in tissue culture media containing alveolar epithelial cells (Chibucos, M. C. et al. An integrated genomic and transcriptomic survey of mucormycosis-causing fungi. Nature communications 7, 12218, doi:10.1038/ncomms12218 (2016)) in order to identify in vivo-specific gene regulation. We will also compare the in vivo RNA-seq data across time points to identify genes whose expression is temporally regulated during infection. Statistical analysis of differential gene expression will be performed using the Bioconductor DESeq package (Anders, S. & Huber, W. Differential expression analysis for sequence count data. Genome Biol 11, 2010-2011 (2010)). A gene will be considered differentially expressed if the absolute fold-change is ≥2.0 and the false discovery rate is ≤0.05.

Absolute expression analysis. There is an increasing body of evidence suggesting that high absolute expression levels and changes in absolute expression may provide more biological insight than examining fold changes in gene expression. In the fungal pathogenesis field, this is supported by independent studies that functionally followed-up C. albicans Nano-string and RNA-seq based in vivo expression data (Bruno, V. M. et al. Transcriptomic analysis of vulvovaginal candidiasis identifies a role for the NLRP3 inflammasome. MBio 6, doi:10.1128/mBio.00182-15 (2015); Xu, W. et al. Activation and Alliance of Regulatory Pathways in C. albicans during Mammalian Infection. PLoS Biology 13, e1002076, doi:10.1371/journal.pbio.1002076 (2015)). RPKM values are a standard way of expressing absolute expression values based on RNA-seq data (Mortazavi, A., Williams, B. A., McCue, K., Schaeffer, L. & Wold, B. Mapping and quantifying mammalian transcriptomes by RNA-Seq. Nat Methods 5,621-628, doi:10.1038/nmeth.1226 [pii] (2008)). To this end, we will use the RPKM values for each gene in each in vivo condition to generate ranked lists of genes that are the most highly expressed. Hierarchical clustering of normalized RPKMs will be performed, as we have previously described (Bruno, V. M. et al. Comprehensive annotation of the transcriptome of the human fungal pathogen Candida albicans using RNA-seq. Genome Research 20, 1451-1458, doi:10.1101/gr.109553.110 (2010)) to further identify coordinated rank changes. The transcriptome profiles from the different datasets will be assembled into networks of co-regulated genes using WGCNA (Langfelder, P. & Horvath, S. WGCNA: an R package for weighted correlation network analysis. BMC Bioinformatics 9, 1471-2105 (2008)) and used for prioritization along with RPKM ranking and comparative genomics.

2B. Assess the role of select Mucorales ORFs during in vitro infection.

RNAi select genes in Mucorales. We will abrogate the function of select R. delemar ORFs by using RNAi (Ibrahim, A. S. et al. The high affinity iron permease is a key virulence factor required for Rhizopus oryzae pathogenesis. Mol Microbiol 77, 587-604, doi:MMI7234 [pii] 10.1111j.1365-29580.2010.07234.x), which is a stable and effective method of silencing R. delemar genes (Ibrahim, A. S. et al. The high affinity iron permease is a key virulence factor required for Rhizopus oryzae pathogenesis. Mol Microbiol 77, 587-604, doi:10.1111/j.1365-2958.2010.07234.xMMI7234 [pii] (2010)). A 450 bp fragment from the target gene ORF will be PCR amplified and cloned as an inverted repeat under control of the Rhizopus expression vector pRNAi-pdc-intron which contains the pyrF as a selection marker (Mertens, J. A., Skory, C. D. & Ibrahim, A. S. Plasmids for expression of heterologous proteins in Rhizopus oryzae. Arch Microbiol 186, 41-50 (2006)). The resulting plasmids will be transformed into a R. delemar pyrF mutant using the biolistic delivery system (BioRad) and transformants will be selected on minimal media lacking uracil (Skory, C. D. Homologous recombination and double-strand break repair in the transformation of Rhizopus oryzae. Mol Genet Genomics 268, 397-406 (2002)). At least two independently generated transformants from each intended genotype will be confirmed by using Southern blotting and gene expression (Ibrahim, A. S. et al. The high affinity iron permease is a key virulence factor required for Rhizopus oryzae pathogenesis. Mol Microbiol 77, 587-604, doi:MMI7234 [pii] 10.1111/j.1365-2958.2010.07234.x). In the assays below, all strains will be compared to R. delemar M16 transformed with an integrated empty plasmid in the assays below.

Fungal adherence, endocytosis and damage of A549 cells. Each of the RNAi strains will be tested for adherence, endocytosis and the ability to induce damage as describe in Aim 1A (Liu, M. et al. The endothelial cell receptor GRP78 is required for mucormycosis pathogenesis in diabetic mice. J Clin Invest 120, 1914-1924, doi:42164 [pii]10.1172/JCI42164; Gebremariam, T. et al. CotH3 mediates fungal invasion of host cells during mucormycosis. J Clin Invest 124, 237-250, doi:71349 [pii] 10.1172/JCI71349).

Growth rate determination. We do not plan to analyze mutants that grow slowly in vivo. Mutations with slow growth may reduce virulence simply as a consequence of poor growth. Therefore, characterization of slow growing mutants would be of limited intellectual or applied value in the context of the generally accepted paradigms. Therefore, we will test the growth rates of all mutants at 37° C. prior to testing their virulence in mice.

2C. Examine the contribution of select Mucorales ORFs to virulence in vivo. In this subpart, select mutants (up to 10) will be tested for virulence in the neutropenic mouse model as described in Aim 1. Priority for testing the mutants (with similar growth rates) will be given to those with the most profound in vitro phenotype (e.g. reduced invasion/damage). The virulence of the generated R. delemar mutants will be compared with empty plasmid transformants by assessing survival as a primary endpoint as described in Aim 1C. As a secondary endpoint we will assess the tissue fungal burden and histopathological examination of lungs and brains (by qPCR) of mice infected by 5 mutants with the greatest defect in survival. Mice will be sacrificed at early, mid, and late stages of infection)(Ibrahim, A. S. et al. The iron chelator deferasirox protects mice from mucormycosis through iron starvation. J Clin Invest 117, 2649-2657, doi:10.1172/JCI32338 (2007); Ibrahim, A. S., Spellberg, B., Avanessian, V., Fu, Y. & Edwards, J. E., Jr. Rhizopus oryzae adheres to, is phagocytosed by, and damages endothelial cells in vitro. Infect Immun 73, 778-783, doi:73/2/778 [pii] 10.1128/IAI.73.2.778-783.2005 (2005)).

Rigor and Statistical Analysis. All in vitro assays will be done three times and in triplicate. For the in vivo studies, 8 mice/group (equal number of males and females and results stratified by sex) will be used and the experiment repeated to result in a 90% power to detect a 3-day difference in survival by the Log Rank test, or a log difference in tissue fungal burden by Wilcoxon Rank Sum test with Bonferroni correction for post-hoc analysis (α-0.05) (Spellberg, B. et al. Parenchymal organ, and not splenic, immunity correlates with host survival during disseminated candidiasis. Infect Immun 71, 5756-5764 (2003)). The Wilcoxon Rank Sum test will also be used for in vitro non-parametric analysis. Experiments will be conducted in a randomized and blinded fashion. Whenever appropriate, data will be presented as the median±interquartile changes as individual data points. p<0.05 will be significant.

Summary, Significance and Future Direction. Mucormycosis has a remarkably high morbidity/mortality and is on the rise, representing the third most common fungal infection in hematologic malignancy patients (Chamilos, G. et al. Invasive fungal infections in patients with hematologic malignancies in a tertiary care cancer center: an autopsy study over a 15-year period (1989-2003). Haematologica 91, 986-989, doi:03906078_9622 [pii] (2006); Pagano, L. et al. Mucormycosis in hematologic patients. Haematologica 89, 207-214 (2004); Eucker, J., Sezer, O., Graf, B. & Possinger, K. Mucormycoses. Mycoses 44, 253-260 (2001). The current research proposal leverages years of productive collaboration between two research groups that are leading Mucorales research in genomics, transcriptomics, gene manipulation, host-pathogen interactions and animal models to better understand mucormycosis pathogenesis. Our work implicates a role of GFR in virulence of Mucorales and has the potential to repurposing FDA-approved drugs for immediate clinical use as adjunctive therapy. Further, our holistic research proposal using state-of-the-art techniques in part 2 is highly likely to identify new targets/pathways for development of novel therapies (be it immune-based and/or small molecule inhibitors) and/or biomarkers for improving mucormycosis diagnosis.

### Vertebrate Animals

1. Description of Procedures. The animal studies are described in Specific parts 1 and 2. Briefly, mice will be used to assess the therapeutic potential of targeting host growth factor receptors to treat mucormycosis by using tyrosine kinase inhibitors (part 1C). We will also use mice to collect lung samples at different stages of infection to provide RNA samples to study fungal gene expression in vivo by using enriched RNA-seq (part 2A). Finally, we will assess the role of ˜10 genes in the virulence of R. delemar by comparing the virulence of generated mutants with attenuated expression of the targeted genes versus R. delemar transformed with the empty plasmid (Aim 2C). We will use the neutropenic mouse model for these studies. Neutropenia will be induced by intraperitoneal injection of cyclophosphamide (200 mg/kg) and subcutaneous injection of cortisone acetate (500 mg/kg) given on day −2, +3, and +8, relative to infection (Luo, G. et al. Efficacy of liposomal amphotericin B and posaconazole in intratracheal models of murine mucormycosis. Antimicrob Agents Chemother 57, 3340-3347, doi:AAC.00313-13 [pii]10.1128/AAC.00313-13). Mice will be given irradiated feed and sterile water and housed in groups of 5 per cage. To prevent bacterial infection, mice will be given 50 μg/ml enrofloxacin (Baytril; Bayer) in drinking water on day −3 (Luo, G. et al. Efficacy of liposomal amphotericin B and posaconazole in intratracheal models of murine mucormycosis. Antimicrob Agents Chemother 57, 3340-3347, doi:AAC.00313-13 [pii] 10.1 128/AAC.00313-13), then switched to daily treatment of ceftazidime (5 mg) starting on day 0 through Day+13 relative to infection (Sheppard, D. C. et al. Novel inhalational murine model of invasive pulmonary aspergillosis. Antimicrob Agents Chemother 48, 1908-1911 (2004)). Mice will be infected intratracheally with spores of R. delemar, L. corymbifera, or M. circinelloides, after sedation. While pulling the tongue anteriorly to the side with forceps, twenty five □1 of fungal spores (2.5×10⁵ cells) in PBS will be injected through the vocal cords into the trachea with a Fisherbrand Gel-loading tip. Uninfected control mice will be included as negative controls. In addition, in Aim 1C, an arm of mice infected with Mucorales and treated with liposomal amphotericin B (LAmB, 15 mg/kg/d, via tail vein injection) which results in 30-50% cure (Luo, G. et al. Efficacy of liposomal amphotericin B and posaconazole in intratracheal models of murine mucormycosis. Antimicrob Agents Chemother 57, 3340-3347, doi:10.1128/AAC.00313-13 (2013)) will be included to assess the utility of the tyrosine kinase inhibitors as adjunct therapy to current antifungals used to treat the disease. Time to moribundity with moribund mice (characterized by Ruffled/matted fur, Hunched posture, weight loss [e.g. >20%], hypothermia, hyper-/hypoventilation, in ability to eat or drink) showing >1 of these criteria humanely euthanized (see below) will serve as primary endpoint. In some studies, tissue fungal burden in lungs and brains (primary and secondary target organs, respectively) (Luo, G. et al. Efficacy of liposomal amphotericin B and posaconazole in intratracheal models of murine mucormycosis. Antimicrob Agents Chemother 57, 3340-3347, doi:AAC.00313-13 [pii]10.1128/AAC.00313-13) as well as histopathological examination will be performed at selected time points as secondary endpoints. The time of sacrifice for the secondary endpoint will be chosen based on our survival curves and usually conducted a day before death of mice occur in the placebo group so as to avoid biasing the results with the healthy survival effect. In all experiments, eight mice per group will be utilized and the experiment repeated once (a total of 16 mice) to detect a 3-day difference in survival by the Log Rank test, or one log difference in tissue fungal burden (□=0.05).

Over the duration of the project period, approximately 1024, 20-25 gm CD-1 mice (males and females split evenly and results stratified by sex) will be used. These mice will be used as follows:

1C: Explore the therapeutic potential of targeting GFRs in vivo to treat Mucormycosis

Assuming that we will assess up to 5 tyrosine kinase inhibitors that target the three GFRs mentioned in the application.

Survival (monotherapy): 16 mice per arm (two independent experiment)*6 different arms (i.e. a placebo arm and 5 different inhibitors)+10 mice (for untreated control)+6 mice for inoculum determination=112

Tissue fungal burden and Histopath: 16 mice per arm (two independent experiment)*3 different arms (one placebo and 2 lead inhibitors (from the survival studies above)*3 time points (early, mid and late stages of infection)+6 mice for inoculum determination=150

Survival (combination therapy with either LAmB or isavuconazole): 16 mice per arm (two independent experiment)*6 different arms (i.e. placebo, three monotherapy [a selected tyrosine inhibitor from above, LAmB, isavouconazole] and two combination therapy of the selected inhibitor+LAmB or isavuconazole from the above arm and 5 different inhibitors)+10 mice (for untreated control)+6 mice for inoculum determination=112

2A: To define the invasive trascriptome of Mucorales during pulmonary mucormnycosis RNA collection from lungs: 6 mice per arm (two independent experiment)*3 time points (representing early, mid and late stages of infection)+6 mice for inoculum determination=24

Confirmation of gene expression in vivo by RT-PCR. 5 mice per arm*3 time points (representing early, mid and late stages of infection)+3 mice for inoculum determination=18

2C. Examine the Contribution of Select Mucorales ORFS to Virulence In Vivo.

Survival: 16 mice per arm (two independent experiment)*12 different arms (2 empty plasmid transformed R. delemar and 10 selected mutants with RNAi targeting selected genes from in vitro studies)+20 mice (for untreated control over 4 sets of experiments of 5 each)+72 mice for inhaled inoculum determination=284

Tissue fungal burden and Histopath: 16 mice per arm (two independent experiment)*6 different arms (1 empty plasmid transformed R. delemar and 5 selected mutants with RNAi attenuated genes)*3 (time points at early, middle and late stages of infection)+36 mice for inhaled inoculum determination=324

Final total number=112+150+112+24+18+284+324=1024

These mice will be certified pathogen-free and will be purchased from a commercial source.

2. Justification for use of animals. The mouse model is currently the standard method for studying treatment options, host defense, pathogenesis, and diagnosis of mucormycosis infections (reviewed in) (Kamei, K. Animal models of zygomycosis—Absidia, Rhizopus, Rhizomucor, and Cunninghamella. Mycopathologia 152, 5-13 (2001)). Pretreatment of mice with cyclophosphamide (to induce neutropenia) (Ibrahim, A. S. et al. The iron chelator deferasirox protects mice from mucormycosis through iron starvation. J Clin Invest 117, 2649-2657, doi:10.1172/JCI32338 (2007); Ibrahim, A. S., Gebremariam, T., Schwartz, J. A., Edwards, J. E., Jr. & Spellberg, B. Posaconazole mono- or combination therapy for treatment of murine zygomycosis. Antimicrob Agents Chemother 53, 772-775, doi:AAC.01124-08 [pii] 10.1128/AAC.01124-08 (2009)) is frequently done. Also the use of cortisone acetate to immunosuppress mice is widely used (Sheppard, D. C. et al. Novel inhalational murine model of invasive pulmonary aspergillosis. Antimicrob Agents Chemother 48, 1908-1911 (2004); Luo, G. et al. Isavuconazole therapy protects immunosuppressed mice from mucormycosis. Antimicrob Agents Chemother 58, 2450-2453, doi:AAC.02301-13 [pii] 10.1128/AAC.02301-13; Waldorf, A. R., Halde, C. & Vedros, N. A. Murine model of pulmonary mucormycosis in cortisone-treated mice. Sabouraudia 20, 217-224 (1982)). In terms of the route of infection, the intranasal (Ibrahim, A. S. et al. The iron chelator deferasirox protects mice from mucormycosis through iron starvation. J Clin Invest 117, 2649-2657, doi:10.1172/JCI32338 (2007); Waldorf, A. R., Halde, C. & Vedros, N. A. Murine model of pulmonary mucormycosis in cortisone-treated mice. Sabouraudia 20, 217-224 (1982)), intrasinus (Waldorf, A. R., Levitz, S. M. & Diamond, R. D. In vivo bronchoalveolar macrophage defense against Rhizopus oryzae and Aspergillus fumigatus. Journal of Infectious Diseases 150, 752-760 (1984)), intracerebral (Corbel, M. J. & Eades, S. M. Observations on the experimental pathogenicity and toxigenicity of Mortierella wolfii strains of bovine origin. British Veterinary Journal 147, 504-516 (1991)) intravenous (Ibrahim, A. S. et al. The iron chelator deferasirox protects mice from mucormycosis through iron starvation. J Clin Invest 117, 2649-2657, doi:10.1172/JCI32338 (2007)) and intratracheal (Luo, G. et al. Isavuconazole therapy protects immunosuppressed mice from mucormycosis. Antimicrob Agents Chemother 58, 2450-2453, doi:AAC.02301-13 [pii] 10.1128/AAC.02301-13) injections have been described. At present, there is no adequate in vitro alternative to study pathogenesis and evaluate the role of potential therapies against a particular infection. We will use CD-1 mice for these studies because: (a) it is an outbred strain, which mimics the genetic variability in humans; and (b) we have significant experience in using this strain and we already established the majority of the optimal parameters of infection for the tested Mucorales using the intratracheal route (Luo, G. et al. Efficacy of liposomal amphotericin B and posaconazole in intratracheal models of murine mucormycosis. Antimicrob Agents Chemother 57, 3340-3347, doi:AAC.00313-13 [pii] 10.1128/AAC.00313-13; Luo, G. et al. Isavuconazole therapy protects immunosuppressed mice from mucormycosis. Antimicrob Agents Chemother 58, 2450-2453, doi:AAC.02301-13 [pii] 10.1128/AAC.02301-13). Thus, we will only have to perform minimum, and small scale experiments to optimize the described assays. Every effort has been made to obtain the maximum amount of information while reducing to a minimum the number of animals required for these studies. Our sample size in each arm is based on the extensive experience with these kind of studies and on

consultation with a Biostatistician to achieve statistical significance.

3. Minimization of Pain and Distress. Every attempt will be made to treat the mice humanely. For the intratracheal injection model, mice will be sedated by ip injection of 0.2 ml of a mixture of ketamine at 82.5 mg/kg and xylazine at 6 mg/kg (Luo, G. et al. Isavuconazole therapy protects immunosuppressed mice from mucormycosis. Antimicrob Agents Chemother 58, 2450-2453, doi:AAC.02301-13 [pii] 10.1128/AAC.02301-13). The sedated mice will be kept on heat pads which will be pre-warmed to 37° C. as we previously described (Luo, G. et al. Isavuconazole therapy protects immunosuppressed mice from mucormycosis. Antimicrob Agents Chemother 58, 2450-2453, doi:AAC.02301-13 [pii]10.1128/AAC.02301-13). While pulling the tongue anteriorly to the side with forceps, twenty five μl of fungal spores (2.5×10⁵ cells) in PBS will injected through the vocal cords into the trachea with a Fisherbrand Gel-loading tip. This technique resulted in <1.0% injury to the vocal cords of the mice (Luo, G. et al. Isavuconazole therapy protects immunosuppressed mice from mucormycosis. Antimicrob Agents Chemother 58, 2450-2453, doi:AAC.02301-13 [pii]10.1128/AAC.02301-13). The survival and health of the mice will be monitored twice daily. Obviously sick, lethargic mice (as determined for their inability to reach for food and water) will be segregated from the group and euthanized to minimize suffering.

4. Euthanasia. The mice will be euthanized by pentobarbital overdose (210 mg/kg) followed by cervical dislocation, as recommended by the Panel on Euthanasia of the American Veterinary Medical Association.

### Example 3. Inhibition of Progesterone Signaling Decreases Mucorales-Induced Host Cell Damage and Mucorales Endocytosis

Mucormycosis is a life-threatening, invasive infection caused by fungi belonging to the Order Mucorales. Rhizopus species are the most common cause of the disease, responsible for approximately 70% of all cases. During pulmonary mucormycosis, inhaled Mucorales spores must adhere to and invade airway epithelial cells in order to establish infection. The molecular mechanisms that govern this interaction are poorly understood. We performed an unbiased survey of the host transcriptional response during Mucorales infection in an in vitro model of pulmonary mucormycosis using RNA-seq. Network analysis revealed activation of progesterone signaling pathways.

Combining an in vitro model of mucormycosis, transcriptomics, cell biology, and pharmacological approaches, here we provide experimental evidence that Mucorales fungi activate PGR signaling to induce fungal uptake into airway epithelial cells. Furthermore, we demonstrated that inhibition of PGR signaling with existing FDA-approved drugs significantly decreased Mucorales-induced host cell damage and endocytosis of alveolar epithelial cells. We also provide experimental evidence to support that there is convergence of EGFR and PG signaling pathways. These studies enhance our understanding of how Mucorales fungi invade host cells during the establishment of pulmonary mucormycosis and provide a proof-of concept for the repurposing of FDA-approved drugs that target RTK and PGR function.

Lung epithelial cells are among the first host cells to interact with inhaled fungal pathogens during pulmonary infection. Understanding the molecular mechanisms used by Mucorales to interact with host receptors on alveolar epithelial cells is crucial to our understanding of how these fungi invade host tissues and initiate pulmonary infection. Once these interactions are understood, preventive and therapeutic strategies can be developed to potentially block interaction with these receptors or activation of downstream signaling pathways. We performed an unbiased survey of the host transcriptional response Mucorales infection in an in vitro model of pulmonary mucormycosis using transcriptome sequencing (RNA-seq). Network analysis predicted modulation of host progesterone receptor (PGR) signaling during infection of airway alveolar cells with R. delemar, R. oryzae, and M. circinelloides. The previous examples herein discuss the involvement of RTK signaling (specifically EGFR, ErbB2, and PDGFR) in pulmonary mucormycosis. Here, we provide experimental evidence that host PGR signaling also governs invasion of human airway epithelial cells by Mucorales and possibly involves cross talk with the EGFR signaling pathway.

Progesterone (PG) modulates a variety of physiological processes including pregnancy, lung development, sperm function, nervous system function, glucose tolerance, pancreas function, and breast cancer etiology (Garg, D., et al., Progesterone-Mediated Non-Classical Signaling. Trends Endocrinol Metab, 2017. 28(9): p. 656-668; Picard, F., et al., Progesterone receptor knockout mice have an improved glucose homeostasis secondary to beta-cell proliferation. Proc Natl Acad Sci USA, 2002. 99(24): p. 15644-8; Kazmi, N., et al., The role of estrogen, progesterone and aromatase in human non-small-cell lung cancer. Lung Cancer Manag, 2012. 1(4): p. 259-272; Lanari, C. and A. A. Molinolo, Progesterone receptors—animal models and cell signalling in breast cancer. Diverse activation pathways for the progesterone receptor: possible implications for breast biology and cancer. Breast Cancer Res, 2002. 4(6): p. 240-3). Progesterone receptor (PGR) exists as both cytosolic and membrane forms. The cytosolic form of PGR exists as two well-characterized main isoforms, PR-A and PR-B, which are encoded by a single gene but transcribed from two different promoters (Garg, D., et al., Progesterone-Mediated Non-Classical Signaling. Trends Endocrinol Metab, 2017. 28(9): p. 656-668; Thomas, P. and Y. Pang, Membrane progesterone receptors: evidence for neuroprotective, neurosteroid signaling and neuroendocrine functions in neuronal cells. Neuroendocrinology, 2012. 96(2): p. 162-71). Upon binding of progesterone, cytosolic PGR dimerizes, enters the nucleus, and binds to DNA at progesterone response elements (PRE) (Shupnik, M. A., Crosstalk between steroid receptors and the c-Src-receptor tyrosine kinase pathways: implications for cell proliferation. Oncogene, 2004. 23(48): p. 7979-89). These forms are rapidly transported between the cytosol and nucleus and function as ligand-activated transcription factors (Garg, D., et al., Progesterone-Mediated Non-Classical Signaling. Trends Endocrinol Metab, 2017.28(9): p. 656-668). The cytosolic PGRs mediate the classical or genomic PG signaling pathways (Garg, D., et al., Progesterone-Mediated Non-Classical Signaling. Trends Endocrinol Metab, 2017. 28(9): p. 656-668). Specific membrane-bound forms of PGR (isoforms mPRα, mPRβ, mPRγ) have been implicated in mediating non-genomic/non-classical PG action (Garg, D., et al., Progesterone-Mediated Non-Classical Signaling. Trends Endocrinol Metab, 2017. 28(9): p. 656-668; Thomas, P. and Y. Pang, Membrane progesterone receptors: evidence for neuroprotective, neurosteroid signaling and neuroendocrine functions in neuronal cells. Neuroendocrinology, 2012. 96(2): p. 162-71). These membrane forms are more novel and function as novel G protein-coupled receptors ((Garg, D., et al., Progesterone-Mediated Non-Classical Signaling. Trends Endocrinol Metab, 2017. 28(9): p. 656-668; Thomas, P. and Y. Pang, Membrane progesterone receptors: evidence for neuroprotective, neurosteroid signaling and neuroendocrine functions in neuronal cells. Neuroendocrinology, 2012. 96(2): p. 162-71; Fernandes, M. S., J. J. Brosens, and B. Gellersen, Honey, we need to talk about the membrane progestin receptors. Steroids, 2008. 73(9-10): p. 942-52).

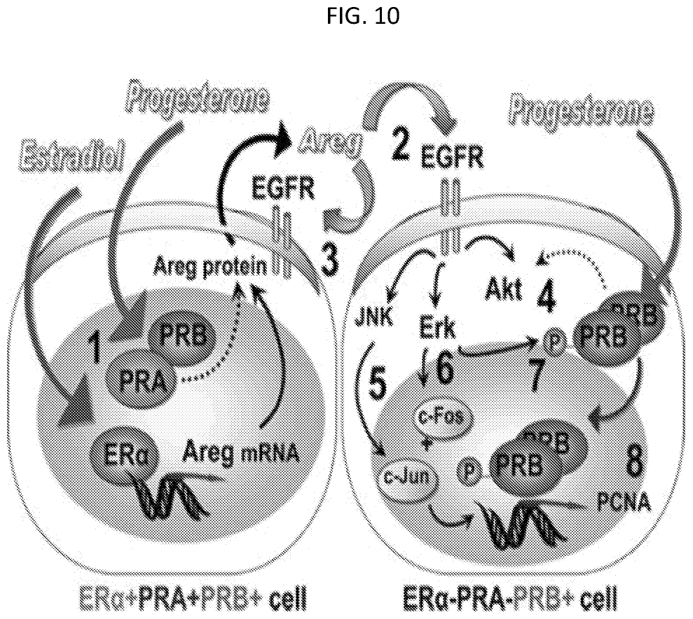

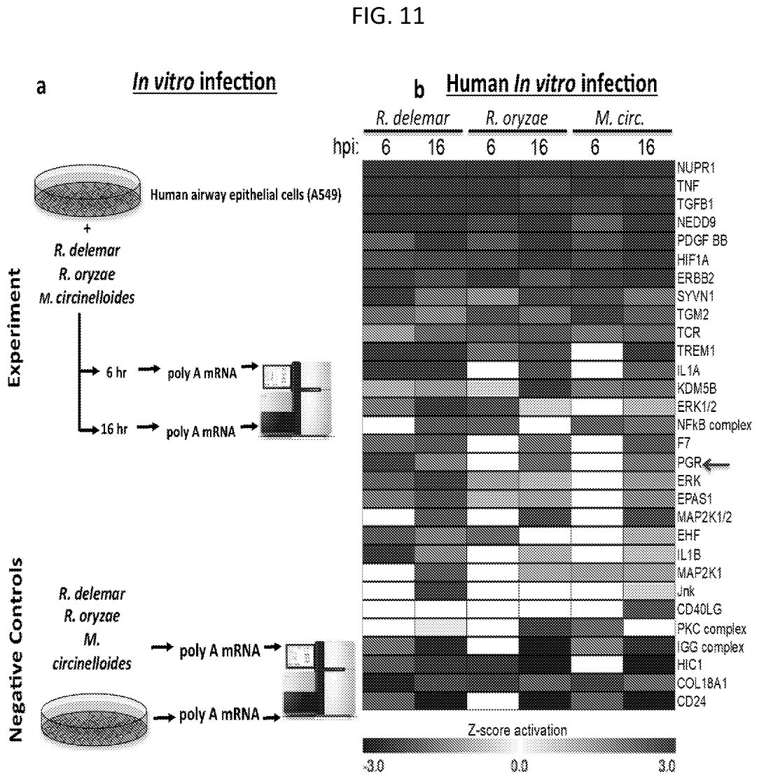

Cross-talk between PGR and EGFR signaling has been reported. Studies in the T47D breast cancer cell line have demonstrated that PG and EGFR signaling are closely interconnected, and that PGR-B appears to be a critical mediator in the cross talk between these two pathways (Anastasia Kariagina, J. X. and S. Z. H. Jeffrey R. Leipprandt, Amphiregulin Mediates Estrogen, Progesterone, and EGFR Signaling in the Normal Rat Mammary Gland and in Hormone-Dependent Rat Mammary Cancers. HORM CANC, 2010: p. 229-244). PGR target genes include key regulators of growth factor signaling pathways including EGFR (Pierson-Mullany, L. K., et al., Cross-talk between growth factor and progesterone receptor signaling pathways: implications for breast cancer cell growth. Breast Dis, 2003. 18: p. 21-31; Lange, E. J. F. a. C. A., Progesterone Receptors Upregulate Wnt-1 To Induce Epidermal Growth Factor Receptor Transactivation and c-Src Dependent Sustained Activation of Erkl/2 Mitogen-Activated Protein Kinase in Breast Cancer Cells. MOLECULAR AND CELLULAR BIOLOGY. 2007: p. 466-480). EGF also activates several intracellular signaling pathways downstream of EGFR that increase transcriptional activity of PGR-B in the presence and absence of ligand (Daniel, A. R., et al., Linkage of progestin and epidermal growth factor signaling: phosphorylation of progesterone receptors mediates transcriptional hypersensitivity and increased ligand-independent breast cancer cell growth. Steroids, 2007. 72(2): p. 188-201). FIG. 10 gives a visual representation of the interaction between the PGR and EGFR pathways showing downstream activation of JNK and ERK signaling, also predicted to be activated during alveolar lung epithelial cell infection with Mucorales and is well-established in the literature to be activated in the host during fungal infection (FIG. 11)(Xin-Ming Jia, B. T., Le-Le Zhu, Yan-Hui Liu, Xue-Qiang Zhao, Sara Gorjestani, Yen-Michael S. Hsu, Long Yang, Jian-Hong Guan, Guo-Tong Xu, Xin Lin, CARD9 mediates Dectin-1-induced ERK activation by linking Ras-GRF1 to H-Ras for antifungal immunity. J Exp Med., 2014. 211(11): p. 2307-2321).

### Results

Transcriptomics

RNA-seq was performed on poly A-enriched RNA isolated from uninfected A549 cells, and A549 cells that were infected with R. delemar, R. orvzae, and M. circinelloides for 6 h or 16 h (Chibucos, M. C., et al., An integrated genomic and transcriptomic survey of mucormycosis-causing fungi. Nat Commun, 2016. 7: p. 12218). Upstream regulator analysis of these data predicted that host PGR signaling was activated during infection based on the differential expression of known PGR downstream transcriptional targets of these pathways (FIG. 11).

We tested the hypothesis that Mucorales invade and cause host cell damage by activating PGR signaling pathways and that blocking this signaling pathway will result in reduced invasion and damage. We were not able to show R. delemar-induced PGR phosphorylation by Western blot despite varying the experimental conditions in several different ways including: a) seeding A549 cells at various cell densities prior to infection, b) starving host cells, c) infecting host cells with various multiplicity of infections (MOIs), d) germinating for various time points, e) infecting over a time course, and f) evaluating various phospho-specific antibodies. Inhibition of PGR inhibits damage and R. delemar internalization.

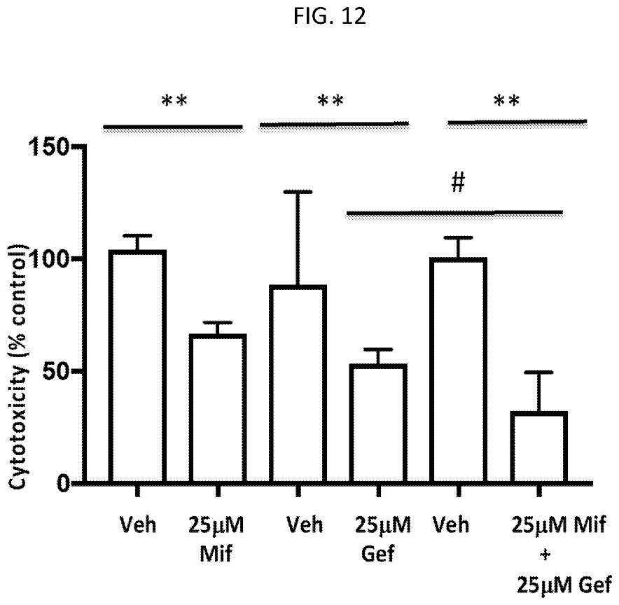

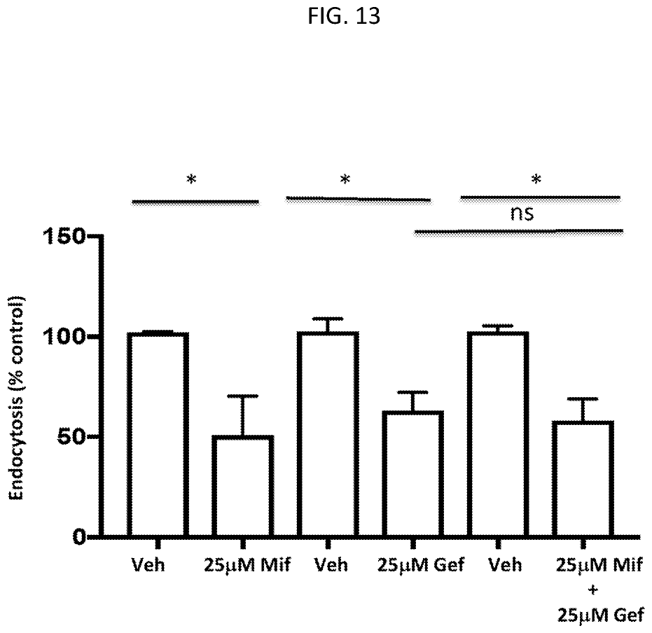

Despite our inability to detect infection-induced phosphorylation of PGR by western blot, we tested the relevance of PGR activation on infection by pretreating airway epithelial cells with an inhibitor of PGR activation, Mifepristone. Mifepristone is structurally related to steroids and strongly binds to PGR and acts as an antagonist of classical PGR signaling (Garg, D., et al., Progesterone-Mediated Non-Classical Signaling. Trends Endocrinol Metab, 2017. 28(9): p. 656-668; Feiteiro, J., et al., Genomic and Nongenomic Effects of Mifepristone at the Cardiovascular Level: A Review. Reprod Sci, 2017. 24(7): p. 976-988). It forms a complex with PGR and its hormone regulator elements in the DNA without stimulating gene transcription [189, 190]. In the presence of Mifepristone, PGR adopts an inactive conformation and preferentially interacts with co-repressors (Feiteiro, J., et al., Genomic and Nongenomic Effects of Mifepristone at the Cardiovascular Level: A Review. Reprod Sci, 2017. 24(7): p. 976-988). We observed a decrease in R. delemar-induced damage and a reduction in R. delemar invasion of alveolar epithelial cells that were treated with the PGR inhibitor Mifepristone (FIGS. 12 and 13). Since it is known that there is some overlap in the EGFR and PGR signaling pathways, we decided to evaluate the effect of inhibiting both receptors on R. delemar-induced damage and endocytosis. Simultaneous treatment of alveoli cells with Mifepristone and Gefitinib (a FDA-approved drug that inhibits EGFR tyrosine kinase signaling) significantly reduced the amount of damage when compared to Gefitinib treatment alone (FIG. 12). However, simultaneous treatment of host cells with Mifepristone and Gefitinib did not significantly reduce the amount of R. delemar endocytosis (FIG. 13). These results are consistent with a model in which PGR and EGFR function in the same pathway to govern Mucorales uptake by airway epithelial cells but not to cause damage.

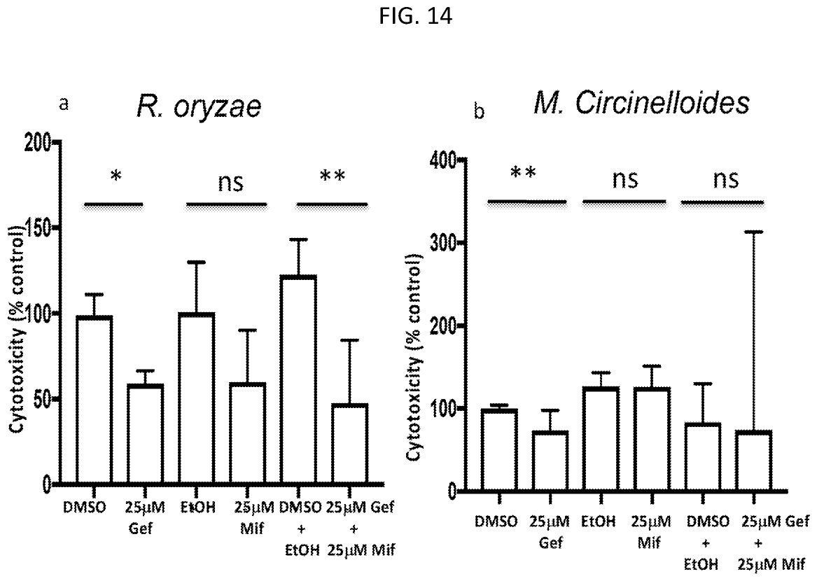

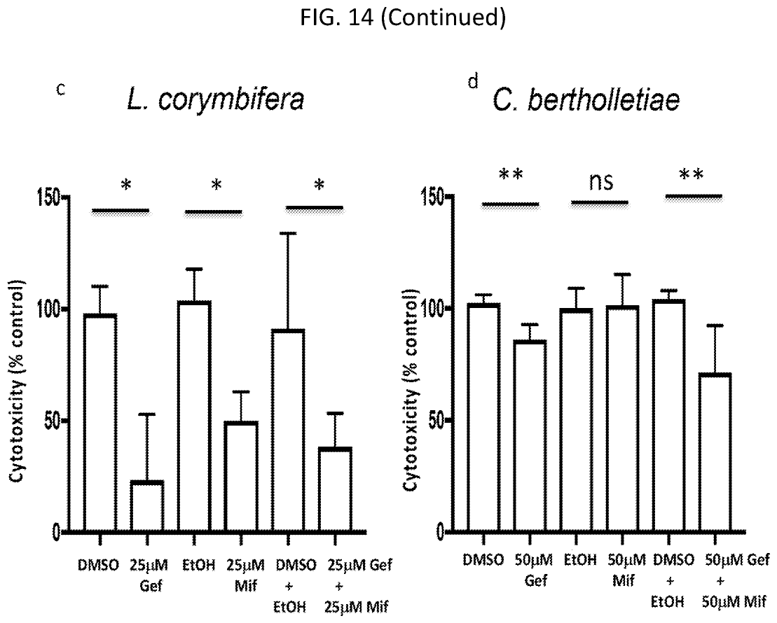

We were also interested in determining if damaged caused by other Mucorales was also significantly decreased by PGR inhibition with Mifepristone alone and in combination with Gefitinib. Once again, we selected a different species of Rhizopus and 3 genera from other Mucorales clades based on our group's previous phylogenetic analysis of Mucorales strains (data not shown) (Chibucos, ibid]). Lictheimia corymbifera-induced damage was significantly decreased by Mifepristone treatment of host cells, while a significant decrease in damage was not seen for R. oryzae, M. circinelloides and C. bertholletiae. The previous discussion states that R. oryzae, R. delemar, M. circinelloides, L. corymbifera, and C. bertholletiae-induced damage is significantly decreased when host cells are treated with Gefitinib. Again, since there is known crosstalk between EGFR and PGR signaling pathways, we were interested in evaluating the effect of dual inhibition of EGFR and PGR with Gefitinib and Mifepristone on host cell damage. Dual inhibition resulted in significantly less damage for R. oryzae, L. corymbifera, and C. bertholletiae. However, it does not appear to be a synergistic effect (FIG. 14) because the damage resulting after combination treatment was not significantly different from the damage resulting after Mifepristone treatment alone.

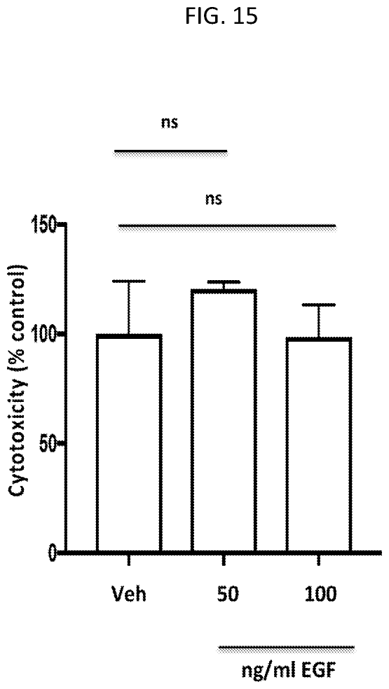

FIG. 10 shows the convergence of the PGR and EGFR signaling pathways where PG activates PGR resulting in Areg (EGFR ligand) release and autocrine and paracrine activation of EGFR. Transactivation of ErbB family members, in some cases, is also mediated by ligands that are cleaved from their proforms in a process called “ectodomain shedding.” We were interested in evaluating if EGF addition during Mucorales infection would augment host cell damage as measured by the LDH assay. When A549 cells were pretreated (and cotreated) with EGF for 1 h prior to 24 h infection with R. delemar, no increase in damage was observed (FIG. 15). This suggests that damage by R. delemar does not occur as a result of surface shedding or increased release of the EGF ligand (FIG. 15). R. delemar infection results in PGR nuclear translocation.

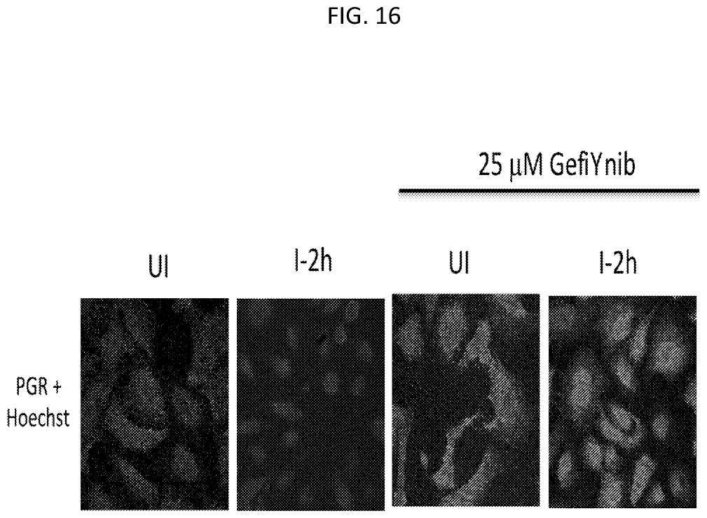

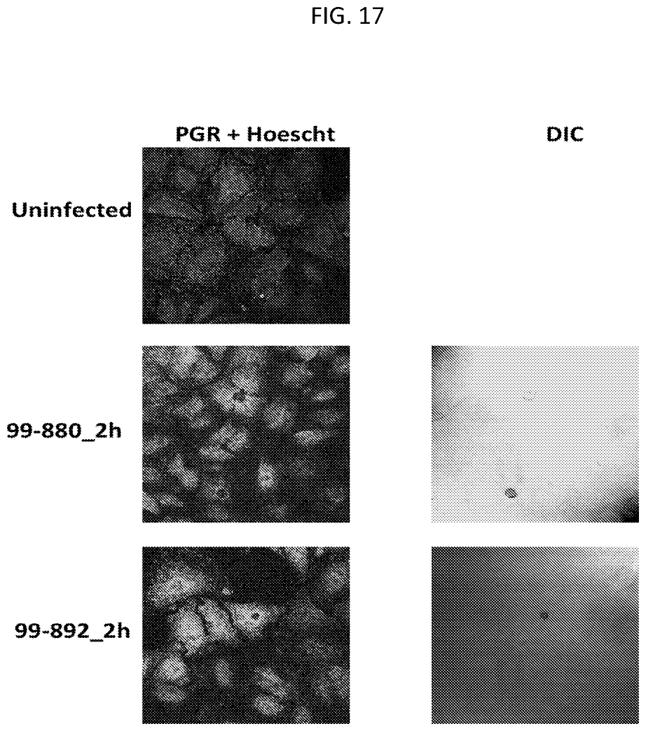

The cytosolic form of PGR is translocated to the nucleus during its activation by its natural ligand, progesterone (Shupnik, M. A., Crosstalk between steroid receptors and the c-Src-receptor tyrosine kinase pathways: implications for cell proliferation. Oncogene, 2004. 23(48): p. 7979-89). When A549 cells were infected with R. delemar for 2 h, we observed translocation of PGR from the cytosol to the nucleus (FIG. 16). We were also interested in seeing if this translocation was Gefitinib sensitive. When A549 cells were treated with Gefitinib followed by infection with R. delemar, translocation of PGR was reduced (FIG. 16). However, PGR translocation was not observed when airway epithelial cells were infected with R. oryzae (FIG. 17).

R. delemar expresses a PGR-like protein

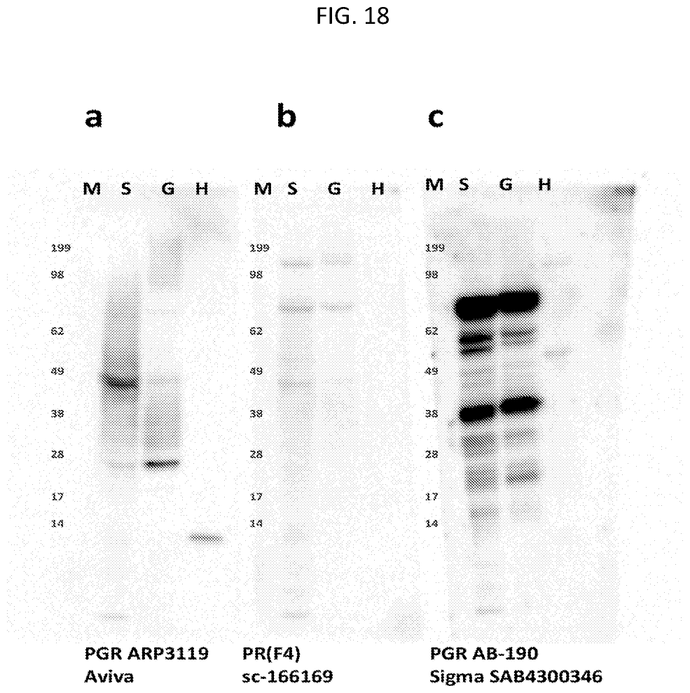

We have western blot data that shows fungal proteins, specifically R. delemar, were reactive with antibodies targeting human cytosolic PGR (FIG. 18). As a follow-up, we wanted to see if we could bioinformatically identify putative PGR-like Mucorales-encoded proteins. The NCBI tool BLASTP (default settings) did not identify any putative human PGR B orthologs in R. delemar. Putative orthologs were defined as having a % identity 2 30%, alignment length ≥100aa, expectation values <0.001, and a bit score ≥50 (Pearson, W. R., An introduction to sequence similarity (“homology”) searching. Curr Protoc Bioinformatics, 2013. Chapter 3: p. Unit3.1). We next wanted to determine if functional regions or domains present in human PGR B were present in Mucorales-encoded proteins by searching the PFAM database. PFAM identified 3 functional domains in human PGR B: Prog receptor (PF02161), Zf-C4 (PF00105), and hormone receptor (PF00104). No fungal proteins in the PFAM database or FungiDB possessed any of these domains.

Both membrane and cytosolic progesterone binding proteins have been described in Mucorales. The characterization and ligand identification of a progesterone receptor in the fungi Sporothrix schenckii has been described. This receptor is described as a membrane receptor belonging to the progesterone-adiponectin receptor (PAQR) family. We were also interested in seeing if an ortholog of this protein was present in Mucorales. The NCBI tool BLASTP (default settings) identified 6 putative Sporothrix schenckii PAQR1 orthologs in R. delemar. Putative orthologs were defined as having a % identity ≥30%, alignment length ≥100aa, expectation values <0.001, and a bit score ≥50 (Pearson, W. R., An introduction to sequence similarity (“homology”) searching. Curr Protoc Bioinformatics, 2013. Chapter 3: p. Unit3.1). We then used PFAM to identify functional domains in the Sporothrix schenckii PAQR1 protein. PFAM identified 1 functional domain: HLyIII (PF03006). The PFAM id was searched against the FungiDB database and 4 Mucor circinelloides f. lusitanicus and 6 Rhizopus delemar genes were identified as having PF03006 present (Table 1). The Mucor circinelloides f. lusitanicus proteins were annotated as predicted membrane proteins containing hemolysin III domains, and the R. delemar proteins were annotated as hypothetical proteins. The Bruno laboratory previously analyzed and annotated the genome of R. delemar 99-880. The FungiDB gene ids for the 99-880 proteins were mapped to the Bruno laboratory's internal ids and annotation (Table 1). The Bruno laboratory identified one of the R. delemar genes as an ADIPOR-like receptor and the other four as uncharacterized. Five putative R. delemar orthologs of Sporothrix schenckii PAQR1 identified by BLASTP were also identified as having Sporothrix schenckii PAQR1 functional domains: IGS-99-880.gene.4086.1, IGS-99-880.gene.8203.1, IGS-99-880.gene.778.1, IGS-99-880.gene.11799.1, IGS-99-880.gene.11034.1.

TABLE 1
Annotation for Mucorales genes containing PFAM functional
domain PF03006.

| FungiDB | | IGS | | |
|---|---|---|---|---|
| Gene ID | Annotation | Gene ID | Annotation | Organism |
| RO3G_02045 | hypothetical | IGS-99- | uncharacterized | R. delemar |
| | protein | 880.gene.4086.1 | | |
| RO3G_02500 | hypothetical | IGS-99- | uncharacterized | R. delemar |
| | protein | 880.gene.778.1 | | |
| RO3G_04590 | hypothetical | ISS-99- | uncharacterized | R. delemar |
| | protein | 880.gene.11799.1 | | |
| RO3G_06498 | hypothetical | IGS-99- | ADIPOR-like receptor CG5315 | R. delemar |
| | protein | 880.gene.11034.1 | | |
| RO3G_09449 | hypothetical | IGS-99- | uncharacterized | R. delemar |
| | protein | 880.gene.8781.1 | | |
| RO3G_14079 | hypothetical | IGS-99- | uncharacterized | R. delemar |
| | protein | 880.gene.8203.1 | | |
| QYA_105913 | Predicted | | | M. |
| | membrane | | | circinelloides f. |
| | proteins, contain | | | lusitanicus |
| | hemolysin III | | | |
| | domain | | | |
| QYA_136039 | Predicted | | | M. |
| | membrane | | | circinelloides f. |
| | proteins, contain | | | lusitanicus |
| | hemolysin III | | | |
| | domain | | | |
| QYA_154832 | Predicted | | | M. |
| | membrane | | | circinelloides f. |
| | proteins, contain | | | lusitanicus |
| | hemolysin III | | | |
| | domain | | | |
| QYA_157113 | Predicted | | | M. |
| | membrane | | | circinelloides f. |
| | proteins, contain | | | lusitanicus |
| | hemolysin III | | | |
| | domain | | | |

### Methods and Materials

### Fungal Strains and Host Cells

R. delemar strain 99-880 (a clinical isolate obtained from a patient with rhino-orbital mucormycosis), R. oryzae strain 99-892, L. corymbifera strain 008-049, M. circinelloides strain NRRL3631, C. bertholletiae strain 175, A. fumigatus MYA4609 (Af293), and A. fumigatus CEA10 were grown on peptone-dextrose agar (PDA) plates for 3 to 5 days at 37° C. Spores were collected in endotoxin-free Dulbecco's phosphate-buffered saline (DPBS), washed with endotoxin-free DPBS, and counted with a hemocytometer to prepare the final inocula. To form germlings, spores were incubated in yeast extract-peptone-dextrose (YPD) with shaking for 1 h at 37° C. Germlings were washed twice with endotoxin-free DPBS. The A549 type II pneumocyte cell line was grown in tissue culture dishes in F-12K medium with L-glutamine plus 10% fetal bovine serum (FBS).

Drugs

Drugs were Obtained for the Indicated Sources.

| | | |
|---|---|---|
| Mifepristone | Sigma | (PGR inhibitor) |
| Gefitinib | Sigma | (EGFR inhibitor) |

Immunoblot of PGR in vitro

Lysates were prepared from resting spores or 5.5 h germlings from R. delemar using 1× Cell Lysis Buffer (Cell Signaling). Lysates were sonicated on ice followed by centrifugation at 10,000 rpm×5 min. Protein concentration was determined using the BCA kit (ThermoFisher) using BSA as a standard. Equal amounts of lysates were then subjected to SDS/PAGE, transferred to PVDF membranes, blocked with 5% BSA, and incubated with anti-PGR antibody (PGR ARP3119 Aviva (recognizes A and B), PR(F4) Santa Cruz 166169 (recognizes A and B), PGR AB-190 Sigma SAB43000346 (recognizes A and B). The immunoblots were developed using enhanced chemiluminescence and imaged with the ChemiDoc (BioRad).

### Measurement of Mucorales-Induced Damage of A549 Cells

R. delemar-induced A549 cell damage was quantified using the Pierce LDH assay, with slight modifications to the manufacturer's protocol. Briefly, A549 cells were grown in 96-well tissue culture plates for 18 to 24 h. Cells were then pretreated for 1 h with inhibitor and infected with 1×10⁶-2×10⁶ germlings suspended in 150 μl F-12K plus 10% FBS. For controls, host cells were incubated with the appropriate amount of diluent used to reconstitute the in parallel. After 24 h of incubation at 37° C., 50 μl of the cell culture supernatant was collected from uninfected, infected, and fungus-only control wells and transferred to a 96-well plate to assay for LDH activity. Lysis buffer was added to all infected wells, and the mixture was incubated for 45 min at 37° C. After lysis, 50 μl of cell culture supernatant was transferred to a 96-well plate and used for the LDH assay kit per the protocol. LDH release was calculated as follows: % cytotoxicity=[(experimental release−fungal cell spontaneous control−host cell spontaneous control)/(host cell maximum control−fungal cell maximum control−host cell spontaneous control)]×100. LDH is a cytosolic enzyme but will be released into the cell culture medium upon cell membrane damage. The amount of extracellular LDH is proportional to the amount of cell damage.

### Measurement of A549 Endocytosis

12-mm glass coverslips in 12 well dishes will be seeded with A549 alveolar epithelial cells. Cells were then pre-treated 1 h with inhibitors. For controls, host cells were incubated with the appropriate amount of diluent used to reconstitute the inhibitor in parallel. Host cells were then infected with 2×10⁶ Rdelemar. After incubation for 3 h, cells were fixed in 3% paraformaldehyde and stained for 1 h with 1% Uvitex, which specifically binds to chitin in the fungal cell wall. After washing with PBS, coverslips were mounted on a glass slide with a drop of ProLong Gold anti-fade reagent (Molecular Probes) and sealed. The total number of cell-associated organisms (i.e., fungi adhering to the monolayer) per high-powered field was determined by phase-contrast microscopy. The same exact field was examined by epifluorescence microscopy, and the number of brightly fluorescent, uninternalized fungi will be determined. The number of endocytosed organisms is calculated by subtracting the number of fluorescent organisms from the total number of visible organisms. At least 400 organisms were counted in at least 15 different fields per coverslip. Experiments were performed in duplicate on separate days. Data will be expressed as median f interquartile range.

### Confocal Microscopy

Translocation of host PGR was visualized using the Zeiss LSM Duo confocal microscopy system. 12-mm glass coverslips in 12 well dishes were seeded with A549 alveolar epithelial cells and infected with 2×10⁵ R. delemar or R. oryzae that have been incubated in F12K+10% FBS at 37° C. for 2 h. After a 2 h incubation at 37° C., cells were washed with HBSS and fixed with 3% paraformaldehyde. Cells were incubated with anti-PGR (Aviva MS-196-P1, recognizes B) for 1 h or overnight in 0.1% BSA for 1 hour. Coverslips were washed and counterstained with Alexa Fluor 546-labeled goat anti-rabbit or Alexa Fluor 488-goat anti-mouse. After washing, the coverslip will be mounted on a glass slide with a drop of ProLong Gold antifade reagent (Molecular Probes) and viewed by confocal fluorescent microscopy.

### Bioinformatics Analysis in Search of PGR Orthologs

Searches to identify putative fungal PGR and PAQR1 orthologs based on protein sequence homology were performed using BLASTP (default settings) (Altschul, S. F., et al., Basic local alignment search tool. J Mol Biol, 1990. 215(3): p. 403-10). Searches to identify functional domains in PGR and PAQR1 were performed using Pfam 32.0 and FungiDB (http://fungidb.org/fungidb/).

### Statistical Analysis

In vitro experiments were performed in triplicate on at least two separate days. Data are expressed as the median f interquartile range. Treatment groups were compared to controls using the Wilcoxon rank sum test.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. A method of treating or preventing mucormycosis in a subject in need thereof, comprising administering to the subject a therapeutically-effective amount of an agent that inhibits a signaling pathway of a receptor selected from the group consisting of epidermal growth factor receptor, platelet-derived growth factor receptor, ErbB2/Her2, progesterone receptor, and a combination thereof.

2. (canceled)

3. (canceled)

4. (canceled)

5. (canceled)

6. (canceled)

7. (canceled)

8. (canceled)

9. (canceled)

10. (canceled)

11. (canceled)

12. The method of claim 1, wherein the agent comprises cetuximab.

13. (canceled)

14. The method of claim 13, wherein the agent comprises trastuzumab.

15. (canceled)

16. (canceled)

17. The method of claim 1, wherein the agent comprises gefitinib, GW2974, or AG1478 (epidermal growth factor receptor phosphorylation inhibitor).

18. (canceled)

19. The method of claim 1, wherein the agent comprises CAS 205254-94-0 (tyrosine kinase inhibitor III) or imatinib.

20. (canceled)

21. The method of claim 1, wherein the agent comprises AG825 or GW2974.

22. (canceled)

23. The method of claim 1, wherein the agent comprises lapatinib.

24. The method of claim 1, wherein cetuximab and gefitinib are administered to the subject.

25. The method of claim 24, wherein the cetuximab and gefitinib are administered to the subject in the same composition.

26. The method of claim 24, wherein the cetuximab and gefitinib are administered to the subject sequentially in separate compositions.

27. (canceled)

28. The method of claim 1, wherein the agent comprises mifepristone.

29. The method of claim 1, wherein a combination of agents are administered.

30. (canceled)

31. (canceled)

32. (canceled)

33. (canceled)

34. (canceled)

35. The method of claim 1, wherein the agent is administered in combination with an effective amount of an antifungal agent.

36. The method of claim 35, wherein the antifungal agent is selected from the group consisting of amphotericin B (AmB), isavuconazole, posaconazole, and combinations thereof.

37. (canceled)

38. A method of inhibiting invasion of an animal cell by a fungal cell comprising administering to the animal cell an effective amount of an agent that inhibits a signaling pathway of a receptor selected from the group consisting of epidermal growth factor receptor, platelet-derived growth factor receptor, ErbB2/Her2, progesterone receptor, and a combination thereof.

39. (canceled)

40. The method of claim 39, wherein the mammalian cell is a human cell.

41. (canceled)

42. (canceled)

43. (canceled)

44. The method of claim 38, wherein the agent inhibits the signaling pathway of the epidermal growth factor receptor.

45. The method of claim 38, wherein the agent inhibits the signaling pathway of the platelet-derived growth factor receptor.

46. The method of claim 38, wherein the agent inhibits the signaling pathway of ErbB2/Her2.

47. The method of claim 38, wherein the agent inhibits the signaling pathway of the progesterone receptor.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)
